(19) United States
(12) Patent Application Publication
(10) Pub. No.: US 2023/0272040 A1
(43) Pub. Date: Aug. 31, 2023

(54) STEALTH CHIMERIC ANTIGEN RECEPTOR AND USE THEREOF IN REDUCING CYTOTOXICITY TOWARDS NORMAL CELLS

(71) Applicant: SHANDONG BOAN BIOTECHNOLOGY CO., LTD., Yantai (CN)

(72) Inventors: Li ZHOU, West Roxbury, MA (US); Michael HARRIS, Medford, MA (US)

(21) Appl. No.: 18/019,179

(22) PCT Filed: Aug. 19, 2021
(86) PCT No.: PCT/CN2021/113420

§ 371 Date: Feb. 1, 2023

Related U.S. Application Data
(60) Provisional application No. 63/067,870, filed on Aug. 19, 2020.

(51) Int. Cl. C07K 14/725; C12N 5/0783; C07K 14/715; C07K 14/705; C07K 14/005; C07K 14/435; A61P 35/00; A61K 39/00

(52) U.S. Cl. CPC C07K 14/7051; C12N 5/0636; C07K 14/7155; C07K 14/70517; C07K 14/70578; C07K 14/705; C07K 14/70532; C07K 14/70521; C07K 14/005; C07K 14/43504; A61P 35/00; A61K 39/4611; A61K 39/4631; A61K 39/464406; A61K 39/464411; A61K 39/464419; A61K 39/464838; A61K 39/464402; A61K 39/4642; C07K 2319/03; C07K 2317/622; C07K 2317/53; C07K 2319/42

(57) ABSTRACT

The present application relates to a chimeric antigen receptor (CAR) which comprises a target-dependent on-switch CAR. The CAR of the invention may reduce cytotoxicity towards normal cells and improve CAR-T safety. CAR molecules were designed using the transmembrane and juxtamembrane motifs of the IL2 receptor β chain (IL2Rβ or IL2Rb), the L ow-Density Lipoprotein Receptor (LDLR), the Seizure 6-like Protein 2 (SEZ6L2), and degradation sequence (PSKFFSQL) of IL2Rβ, which resulted in greatly reduced CAR expression at the cell surface in the absence of target antigen, while retaining downstream activation ability in response to antigen-expressing target cells. In the absence of target antigen, CAR surface expression is undetectable. The present application has shown that primary T cells expressing these surface-unstable CAR variants are able to elicit antigen-dependent target cell killing. By limiting CAR activity in this way, the present application can reduce therapeutic toxicity and T cell exhaustion. Due to its limited detectability in the absence of antigen, the present application refers to this system as a “Stealth CAR”. The present application further relates to compositions, preparation methods and uses of the Stealth CAR of the present application.

This application claims the benefit of U.S. Provisional Patent Application No. 63/067,870, entitled “Target-Dependent On-Switch Chimeric Antigen Receptors”, filed on Aug. 19, 2020; the contents of which are herein incorporated by reference in its entirety.

### TECHNICAL FIELD

The present application relates to the field of biopharmaceuticals, and particularly relates to stealth chimeric antigen receptor (CAR), and related polynucleotides, vectors, compositions, preparation methods and uses thereof.

### BACKGROUND

The initial success of CAR T cell therapy in the treatment of hematological malignancies has heralded rapid growth in a new area of immunotherapeutic treatment strategies. CARs are designer receptors typically with modular activation domains derived from the CD3ζ ITAM motifs and a costimulatory domain, for example, from 4-1BB or CD28. Target recognition by these receptors is usually provided through an scFv domain on the extracellular terminal of the CAR, though alternative approaches have also been designed.

Safety is a major hurdle hindering the development of CAR-T therapeutics. CAR molecules are usually constitutively expressed on the T-cell surface. Thus, when CAR-T cells are injected into patients, they will migrate to different tissues expressing the target antigen often with unintended toxicity and damage to normal tissues alongside tumor cells. In addition, constitutive expression of the CAR molecule promotes tonic signaling and T cell exhaustion.

To date, several types of switches have been developed to control CAR-T activity. Some of these are based on the incorporation of “suicide genes” into the CAR construct. For example, the HSV-TK transgene has been used in adoptive cell therapies to allow for inducible apoptosis of the modified cells on treatment with Ganciclovir. Another approach relies on the use of an inducible Caspase 9 (iCasp9). In these strategies, the therapeutic response is terminated through targeted elimination of the CAR-T cells themselves.

Other switch systems rely on modification of the receptor, for example, adapter-mediated CARs, also known as “universal CARs”. These CAR T cells share a common receptor that can be modified to recognize a variety of tumor targets through linking of the receptor to an adapter molecule that binds both the CAR and the tumor target. Similarly, recruitment of the intracellular signaling domain to the transmembrane component of the CAR via drug-induced dimerization has been proposed as a means of controlling dosing and activation. While current clinically approved CARs are designed to be constitutively active, adapter-dependent CAR T cells can only recognize and kill when the adapter is administered, allowing for titratable and reversible control of the CAR-T cells.

For these types of switches, the CAR expression and functionality is regulated externally through application of small molecule drugs or other therapeutics. There are major challenges for these types of switches including: the availability of existing FDA approved switch molecules that can be applied, the safety profile of the switch molecule, bioavailability, biodistribution, and the complexity of regulation in vivo due to the complex dynamics and distribution of the switch molecules and the CAR-T cells.

Selective pressure on the target cell population brought on through the targeting of a single tumor associated antigen (TAA) can eventually result in downregulation of the target antigen and evasion of the CAR T cell immune response¹ (Ruella & Maus, 2016). Loss of target antigen expression results in unmitigated tumor cell expansion and, ultimately, disease relapse. Studies have reported that on downregulation of the primary TAA, target cells may upregulate a secondary TAA, allowing for a second round of therapeutic intervention² (FIG. 1) (Fousek et al., 2020). Ideally, the secondary TAA should only be targeted once it has been upregulated by the tumor cell population to reduce therapeutic toxicity.

The high-affinity IL2 Receptor is a transmembrane receptor comprised of three distinct, noncovalently associated components: the α, β, and γ chains. It has been shown that in the absence of the α and γ subunits, the β chain is constitutively endocytosed and degraded³ (Hémar et al., 1994).

Analysis of the C-terminal residues of LDLR indicates the presence of the endosomal sorting motif NDXY. Studies indicate that this mediates an indirect endosomal sorting mechanism through interactions with ARH and binding to the AP-2 complex⁴⁻⁵ (Tao et al., 2016; Fasano et al., 2009). SEZ6L2 is characterized by the presence of two endosomal-targeting consensus sequences in its c-terminal region⁶ (Bonifacino et al., 2003).

The present application aims to solve the CAR-T safety problem by providing stealth CAR for reducing cytotoxicity towards normal cells.

### SUMMARY OF THE INVENTION

In the novel CAR system of the present application, expression/stabilization of the CAR molecule is dependent on target antigen availability. When there is no antigen available, there is no detectable CAR on the T cell surface and no CAR function. When the engineered T cell recognizes its target antigen, the CAR is stabilized and signaling can be initiated. This is a target dependent on-switch. By modulating CAR expression in response to antigen, the present application can reduce CAR T cell toxicity and exhaustion. Since the CAR is “invisible” when there is no antigen, the inventors of the present application have elected to name it, “Stealth CAR”. Stealth CAR can be applied in mono- and dual-specific CAR settings, as well as in autologous and allogeneic settings. It can be applied in the context of T cells, NK cells, gamma delta T cells, and iPSC-derived T cells.

The endocytic stealth CAR system can also be applied to address a particular challenge facing CAR T therapies, that is, “on-target, off-tumor” effects. As TAAs are often upregulated on tumor tissues, but also expressed at lower levels on normal tissues, CAR T therapy can have the unwanted effect of targeting non-cancerous cells. By using a CAR that is stabilized by antigen expression, cells expressing high levels of the target antigen (e.g., tumor cells) provide a stronger activation for our CAR system. Thus, the present application can promote tumor-specific killing by CAR T cells, while minimizing the effect on normal tissue (FIG. 2). In a combined model, both CARs may be of the endocytic variety, minimizing on-target, off-tumor effects and addressing antigen escape.

By applying stealth CAR in a dual-specific setting, the present application expects the system could be used to address the issue of antigen escape. By expressing a CAR variant that is only transiently stable at the cell surface, the present application provides a mechanism to reduce collateral targeting of, and damage to, normal tissue. Upregulation of the secondary target antigen on the tumor cells provides a stronger signal and promotes CAR surface stability and downstream activation. By targeting the primary TAA with one CAR and the secondary TAA with a second, stealth CAR, the present application can elicit a strong initial tumor response and targeted secondary response only once the secondary TAA has been upregulated.

The inventors of the present application have designed and prepared several CAR variants with modified transmembrane and juxtamembrane sequences which facilitate the intracellular trafficking of receptors. These are derived from the IL2 Receptor β chain (IL2Rβ or IL2Rb), the Low-Density Lipoprotein Receptor (LDLR), and the human Seizure 6-like Protein 2 (SEZ6L2). The amino acid motifs in the transmembrane and intracellular sequences of these receptors promote intracellular trafficking via the endosome pathway.

The present application provides novel stealth CAR for reducing cytotoxicity towards normal cells and improving CAR-T safety, as well as reducing inflammatory cytokine and tonic signaling caused by constitutive CAR surface expression. The present application also provides dual CARs comprising the stealth CAR and a second CAR, and related nucleic acids, vectors, host cells and pharmaceutical compositions which comprise a polynucleotide encoding the stealth CAR or the dual CAR. The present application further provides a method of treating disease in a subject in need thereof, comprising administering to the subject an effective amount of the pharmaceutical composition, a method of reducing the cytotoxicity of a CAR-T cell towards normal cells, and a method of producing a CAR-T cell with reduced cytotoxicity towards normal cells.

In one aspect, the application provides a chimeric antigen receptor (CAR) comprising:
(1) an extracellular ligand-binding domain comprising a single chain variable fragment (scFv) specifically binding to a predetermined antigen; wherein preferably, the predetermined antigen is a tumor-associated antigen (TAA); more preferably, the TAA is selected from one or more of: CEA, Claudin 18.2, CGC3, Receptor tyrosine kinase-like Orphan Receptor 1 (ROR1), CD38, CD19, CD20, CD22, BCMA, CAIX, CD446, CD13, EGFR, EGFRvIII, EpCam, GD2, EphA2, HER1, HER2, ICAM-1, IL13Ra2, Mesothelin, MUC1, MUC16, NKG2D, PSCA, NY-ESO-1, MART-1, WT1, MAGE-A10, MAGE-A3, MAGE-A4, EBV, NKG2D, PD1, PD-L1, CD25, IL-2, and CD3;
(2) a transmembrane (tm) linking juxtamembrane (jm) domain;
wherein the transmembrane linking juxtamembrane domain comprises an IL2 Receptor β chain (IL2Rβ) transmembrane domain and an IL2Rβ juxtamembrane domain, and the transmembrane linking juxtamembrane domain is adjacent to IL2Rβ degradation sequence (DT); wherein preferably, the IL2Rβ degradation sequence is at the C-terminal of the transmembrane linking juxtamembrane domain; wherein preferably, the IL2Rβ degradation sequence comprises an amino acid sequence that is at least about 95%, 96%, 97%, 98%, 99% or 100% identical to the amino acid sequence represented by SEQ ID NO: 2;
wherein the transmembrane linking juxtamembrane domain comprises a Low-Density Lipoprotein Receptor (LDLR) transmembrane domain and an LDLR juxtamembrane domain; or wherein the transmembrane linking juxtamembrane domain comprises a Seizure 6-like Protein 2 (SEZ6L2) transmembrane domain and a SEZ6L2 juxtamembrane domain;
(3) an intracellular domain; wherein preferably, the intracellular domain comprises a signaling domain; more preferably, the signaling domain comprises one or more signaling domains selected from the group consisting of a 4-1BB signaling domain, a CD28 signaling domain and a CD3ζ signaling domain.

In a further aspect, the CAR comprises from N-terminal to C-terminal: TAA scFv-CD8Hinge-IL2Rβ tm jm DT-4-1BB-CD3ζ, TAA scFv-CD8Hinge-IL2Rβ tm jm DT-CD28-CD3ζ, TAA scFv-CD8Hinge-LDLR tm jm-4-1BB-CD3ζ, or TAA scFv-CD8Hinge-SEZ6L2 tm jm-4-1BB-CD3ζ

In a further aspect, the TAA scFv is selected from one or more of CEA scFv, Claudin 18.2 scFv and HER2 scFv; more preferably, CEA scFv is MN14op CEA scFv, or Claudin 18.2 scFv is 841 Claudin 18.2 scFv; most preferably, MN14op CEA scFv, 841 Claudin 18.2 scFv, or HER2 scFv comprises an amino acid sequence that is at least about 95%, 96%, 97%, 98%, 99% or 100% identical to the amino acid sequence represented by SEQ ID NO. 5, 30, or 31 respectively.

In a further aspect, the N-terminal of the CAR further contains a Leader Sequence and/or a HA Sequence.

In a further aspect, the CAR comprises from N-terminal to C-terminal:
HA-MN14op CEA scFv-CD8Hinge-IL2Rβ tm jm DT-4-1BB-CD3ζ,
HA-MN14op CEA scFv-CD8Hinge-IL2Rβ tm jm DT-4-1BB-CD3ζ-P2A-GFP,
HER2 scFv-CD8Hinge-IL2Rβ tm jm DT-4-1BB-CD3ζ-P2A-GFP,
HER2 scFv-CD8Hinge-IL2Rβ tm jm DT-CD28-CD3ζ-P2A-GFP,
MN14op CEA scFv-CD8Hinge-IL2Rβ tm jm DT-4-1BB-CD3ζ-P2A-GFP,
MN14op CEA scFv-CD8Hinge-LDLR tm jm-4-1BB-CD3ζ-P2A-GFP,
MN14op CEA scFv-CD8Hinge-SEZ6L2 tm jm-4-1BB-CD3ζ-P2A-GFP,
841 Claudin 18.2 scFv-CD8Hinge-SEZ6L2 tm jm-4-1BB-CD3ζ-P2A-GFP, or
HER2 scFv-CD8Hinge-SEZ6L2 tm jm-4-1BB-CD3ζ-P2A-GFP, optionally the CAR does not include P2A-GFP and/or HA;

In a further aspect, the CAR comprises an amino acid sequence that is at least about 95%, 96%, 97%, 98%, 99% or 100% identical to the amino acid sequence represented by SEQ ID NO. 9, 14, 15, 17, 18, 19, 21, 22 or 24 respectively, optionally the amino acid sequence of CAR does not include the amino acid sequence of P2A-GFP, Leader Sequence, and/or HA.

In a further aspect, the CAR comprises hinge domain; preferably the hinge domain comprises CD8 or CD28 hinge derived from the extracellular region, or IgG hinge

In another aspect, the application also provides a chimeric antigen receptor (CAR) comprising:
(1) an extracellular ligand-binding domain comprising single chain variable fragment (scFv) specifically binding to a predetermined antigen; wherein preferably, the predetermined antigen is a tumor-associated antigen (TAA); more preferably, the TAA is selected from one or more of: CEA, Claudin 18.2, CGC3, Receptor tyrosine kinase-like Orphan Receptor 1 (ROR1), CD38, CD19, CD20, CD22, BCMA, CAIX, CD446, CD13, EGFR, EGFRvIII, EpCam, GD2, EphA2, HER1, HER2, ICAM-1, IL13Ra2, Mesothelin, MUC1, MUC16, NKG2D, PSCA, NY-ESO-1, MART-1, WT1, MAGE-A10, MAGE-A3, MAGE-A4, EBV, NKG2D, PD1, PD-L1, CD25, IL-2, and CD3;
(2) a transmembrane domain, and
(3) a cytoplasmic segment comprising an IL2Rβ degradation sequence (DT) and at least one signaling domain; wherein preferably, the IL2Rβ degradation sequence comprises an amino acid sequence that is at least about 95%, 96%, 97%, 98%, 99% or 100% identical to the amino acid sequence represented by SEQ ID NO: 2; wherein preferably, the IL2Rβ degradation sequence is at the C-terminal of the cytoplasmic segment.

In a further aspect, the CAR comprises from N-terminal to C-terminal: TAA scFv-CD8Hinge-CD8 tm-4-1BB-CD3ζ-IL2R DT.

In a further aspect, the N-terminal of the CAR further contains Leader Sequence and/or HA Sequence.

In a further aspect, the TAA scFv is CEA scFv; more preferably, the TAA scFv is MN14op CEA scFv; most preferably, MN14op CEA scFv comprises an amino acid sequence that is at least about 95%, 96%, 97%, 98%, 99% or 100% identical to the amino acid sequence represented by SEQ ID NO. 5; most preferably, the CAR comprises an amino acid sequence that is at least about 95%, 96%, 97%, 98%, 99% or 100% identical to the amino acid sequence represented by SEQ ID NO. 8 or 16 respectively, optionally the amino acid sequence of CAR does not include the amino acid sequence of P2A-GFP, Leader Sequence, and/or HA.

In a further aspect, the transmembrane (tm) of the IL2Rβ, LDLR, or SEZ6L2 comprises an amino acid sequence that is at least about 95%, 96%, 97%, 98%, 99% or 100% identical to the amino acid sequence represented by SEQ ID NO. 1, 38 or 40 respectively;
the juxtamembrane (jm) of the IL2Rβ, LDLR, or SEZ6L2 comprises an amino acid sequence that is at least about 95%, 96%, 97%, 98%, 99% or 100% identical to the amino acid sequence represented by SEQ ID NO. 4, 39 or 41 respectively;
the transmembrane (tm) linking juxtamembrane (jm) of the IL2Rβ, LDLR, or SEZ6L2 comprises an amino acid sequence that is at least about 95%, 96%, 97%, 98%, 99% or 100% identical to the amino acid sequence represented by SEQ ID NO. 27, 28, or 29 respectively;
the MN14op CEA scFv, 841 Claudin 18.2 scFv, HER2 scFv, PD-L1 scFv, HA, CD8Hinge, CD3ζ, 4-1BB, CD28, CD8 tm, GFP, or Leader Sequence comprises an amino acid sequence that is at least about 95%, 96%, 97%, 98%, 99% or 100% identical to the amino acid sequence represented by SEQ ID NO. 5, 30, 31, 32, 33, 34, 35, 36, 37, 42, 45 or 46 respectively; and/or
the P2A comprises an amino acid sequence that is at least about 95%, 96%, 97%, 98%, 99% or 100% identical to the amino acid sequence represented by SEQ ID NO. 43 or 44.

In a further aspect, the application provides a chimeric antigen receptor (CAR) comprising:
(1) an extracellular ligand-binding domain comprising a single chain variable fragment (scFv) specifically binding to a predetermined antigen; (2) a transmembrane (tm) linking juxtamembrane (jm) domain;
wherein the transmembrane linking juxtamembrane domain comprises an IL2 Receptor β chain (IL2Rβ) transmembrane domain and an IL2Rβ juxtamembrane domain, and the transmembrane linking juxtamembrane domain is adjacent to IL2Rβ degradation sequence (DT);
wherein preferably, the IL2Rβ degradation sequence is at the C-terminal of the transmembrane linking juxtamembrane domain; wherein preferably, the IL2Rβ degradation sequence comprises an amino acid sequence that is at least about 95%, 96%, 97%, 98%, 99% or 100% identical to the amino acid sequence represented by SEQ ID NO: 2;
wherein the transmembrane linking juxtamembrane domain comprises a Low-Density Lipoprotein Receptor (LDLR) transmembrane domain and an LDLR juxtamembrane domain; or
wherein the transmembrane linking juxtamembrane domain comprises a human Seizure 6-like Protein 2 (SEZ6L2) transmembrane domain and a SEZ6L2 juxtamembrane domain;
the transmembrane (tm) of the IL2Rβ, LDLR, or SEZ6L2 comprises an amino acid sequence that is at least about 95%, 96%, 97%, 98%, 99% or 100% identical to the amino acid sequence represented by SEQ ID NO. 1, 38 or 40 respectively;
the juxtamembrane (jm) of the IL2Rβ, LDLR, or SEZ6L2 comprises an amino acid sequence that is at least about 95%, 96%, 97%, 98%, 99% or 100% identical to the amino acid sequence represented by SEQ ID NO. 4, 39 or 41 respectively;
(3) an intracellular domain.

In a further aspect, the application also provides a chimeric antigen receptor (CAR) comprising:
(1) an extracellular ligand-binding domain comprising single chain variable fragment (scFv) specifically binding to a predetermined antigen;
(2) a transmembrane domain, and
(3) a cytoplasmic segment comprising an IL2Rβ degradation sequence (DT) and at least one signaling domain; the IL2R degradation sequence comprises an amino acid sequence that is at least about 95%, 96%, 97%, 98%, 99% or 100% identical to the amino acid sequence represented by SEQ ID NO: 2; the IL2Rβ degradation sequence is at the C-terminal of the cytoplasmic segment.

In a specific embodiment, the CAR comprising IL2Rβ transmembrane (tm), juxtamembrane (jm), and degradation sequence (DT), from N-terminal to C-terminal, comprises:
(1) MLB003 CAR: HA-MN14op CEA scFv-CD8Hinge-IL2Rβ tm jm DT-4-1BB-CD3ζ (the amino acid sequence is represented by SEQ ID NO. 9);
(2) MLB013 CAR: HA-MN14op CEA scFv-CD8Hinge-IL2Rβ tm jm DT-4-1BB-CD3ζ-P2A-GFP (the amino acid sequence is represented by SEQ ID NO. 15);
(3) MLB020 CAR: MN14op CEA scFv-CD8Hinge-IL2Rβ tm jm DT-4-1BB-CD3ζ-P2A-GFP (the amino acid sequence is represented by SEQ ID NO. 14);
(4) MLB038 CAR: HER2 scFv-CD8Hinge-IL2Rβ tm jm DT-4-1BB-CD3ζ-P2A-GFP (the amino acid sequence is represented by SEQ ID NO. 21); or
(5) MLB039 CAR: HER2 scFv-CD8Hinge-IL2Rβ tm jm DT-CD28-CD3ζ-P2A-GFP (the amino acid sequence is represented by SEQ ID NO. 22);
optionally the CAR does not include P2A-GFP, Leader Sequence, and/or HA.

In a specific embodiment, the CAR comprising LDLR transmembrane (tm) and juxtamembrane (jm), from N-terminal to C-terminal, comprises:
(1) MLB048 CAR: MN14op CEA scFv-CD8Hinge-LDLR tm jm-4-1BB-CD3ζ-P2A-GFP (the amino acid sequence is represented by SEQ ID NO. 18);
optionally the CAR does not include P2A-GFP, Leader Sequence, and/or HA.

In a specific embodiment, the CAR comprising SEZ6L2 transmembrane (tm) and juxtamembrane (jm), from N-terminal to C-terminal, comprises:
(1) MLB047 CAR: MN14op CEA scFv-CD8Hinge-SEZ6L2 tm jm-4-1BB-CD3ζ-P2A-GFP (the amino acid sequence is represented by SEQ ID NO. 17);
(2) MLB054 CAR: 841 Claudin 18.2 scFv-CD8Hinge-SEZ6L2 tm jm-4-1BB-CD3ζ-P2A-GFP (the amino acid sequence is represented by SEQ ID NO. 19); or
(3) MLB080 CAR: HER2 scFv-CD8Hinge-SEZ6L2 tm jm-4-1BB-CD3ζ-P2A-GFP (the amino acid sequence is represented by SEQ ID NO. 24);
optionally the CAR does not include P2A-GFP, Leader Sequence, and/or HA.

In a specific embodiment, the CAR comprising cytoplasmic segment comprising an IL2Rβ degradation sequence (DT) and at least one signaling domain, from N-terminal to C-terminal, comprises:
(1) MLB025 CAR: MN14op CEA scFv-CD8Hinge-CD8 tm-4-1BB-CD3ζ-IL2R DT-P2A-GFP (the amino acid sequence is represented by SEQ ID NO. 16); or
(2) MLB002 CAR: HA-MN14op scFv-CD8Hinge-CD8 tm-4-1BB-CD3ζ-IL2R DT (the amino acid sequence is represented by SEQ ID NO. 8);
optionally the CAR does not include P2A-GFP, Leader Sequence, and/or HA.

In another aspect, the application also provides a dual CAR comprising: a first CAR according to any of the above-described embodiments, and a second CAR comprising:
(1) an extracellular ligand-binding domain comprising scFv specifically binding to a predetermined antigen;
(2) a transmembrane domain; wherein preferably, the transmembrane domain is CD8 transmembrane domain;
(3) an intracellular domain; wherein preferably, the intracellular domain comprises signaling domain; more preferably, the signaling domain comprises one or more signaling domains selected from the group consisting of a 4-1BB signaling domain, a CD28 signaling domain and a CD3ζ signaling domain;
wherein the first CAR targets an antigen and the second CAR targets another antigen.

In a further aspect, the first CAR and the second CAR is linked by P2A.

In a further aspect, the P2A comprises an amino acid sequence that is at least about 95%, 96%, 97%, 98%, 99% or 100% identical to the amino acid sequence represented by SEQ ID NO. 43 or 44.

In a further aspect, the dual CAR comprises N-terminal to C-terminal:

TAA scFv-CD8Hinge-IL2Rβ tm jm DT-4-1BB-CD3ζ-P2A-another TAA scFv-CD8Hinge-CD8 tm-CD28-CD3ζ, TAA scFv-CD8Hinge-CD8 tm-4-1BB-CD3ζ-P2A-another TAA scFv-CD8Hinge-IL2Rβ tm jm DT-CD28-CD3ζ, TAA scFv-CD8Hinge-LDLR tm jm-4-1BB-CD3ζ-P2A-another TAA scFv-CD8Hinge-CD8 tm-CD28-CD3ζ, TAA scFv-CD8Hinge-CD8 tm-4-1BB-CD3ζ-P2A-another TAA scFv-CD8Hinge-LDLR tm jm-CD28-CD3ζ, TAA scFv-CD8Hinge-SEZ6L2 tm jm-4-1BB-CD3ζ-P2A-another TAA scFv-CD8Hinge-CD8 tm-CD28, or TAA scFv-CD8Hinge-CD8 tm-4-1BB-CD3ζ-P2A-another TAA scFv-CD8Hinge-SEZ6L2 tm jm-CD3ζ.

In a further aspect, the dual CAR comprises N-terminal to C-terminal: 841 Claudin 18.2 scFv-CD8Hinge-SEZ6L2 tm jm-4-1BB-CD3ζ-P2A-PD-L1 scFv-CD8Hinge-CD8 tm-CD28-(G₄S)₂-GFP, 841 Claudin18.2 scFv-CD8Hinge-CD8 tm-4-1BB-CD3ζ-P2A-HER2 scFv-IL2Rβ tm jm DT-CD28-CD3ζ, or 841 Claudin18.2 scFv-CD8Hinge-CD8 tm-4-1BB-CD3ζ-P2A-HER2 scFv-SEZ6L2 tm jm-CD3ζ respectively, optionally the dual CAR does not include (G₄S)₂-GFP; more preferably, 841 Claudin 18.2 scFv, HER2 scFv, or PD-L1 scFv comprises an amino acid sequence that is at least about 95%, 96%, 97%, 98%, 99% or 100% identical to the amino acid sequence represented by SEQ ID NO. 30, 31, or 32 respectively; most preferably, the CAR comprises an amino acid sequence that is at least about 95%, 96%, 97%, 98%, 99% or 100% identical to the amino acid sequence represented by SEQ ID NO. 20, 25 or 26 respectively, optionally the amino acid sequence of CAR does not include the amino acid sequence of (G₄S)₂-GFP and/or Leader Sequence.

In a specific embodiment, the dual CAR, from N-terminal to C-terminal, comprises:
(1) MLB055 CAR: 841 Claudin 18.2 scFv-CD8Hinge-SEZ6L2 tm jm-4-1BB-CD3ζ-P2A-PD-L1 scFv-CD8Hinge-CD8 tm-CD28-(G4S)2-GFP (the amino acid sequence is represented by SEQ ID NO. 20);
(2) MLB040 CAR: 841 Claudin18.2 scFv-CD8Hinge-CD8 tm-4-1BB-CD3ζ-P2A-HER2 scFv-IL2Rβ tm jm DT-CD28-CD3ζ (the amino acid sequence is represented by SEQ ID NO. 25); or
(3) MLB108 CAR: 841 Claudin18.2 scFv-CD8Hinge-CD8 tm-4-1BB-CD3ζ-P2A-HER2 scFv-SEZ6L2 tm jm-CD3ζ (the amino acid sequence is represented by SEQ ID NO. 26);
optionally the dual CAR does not include (G₄S)₂-GFP and/or Leader Sequence.

In another aspect, the application also provides the nucleic acid comprising a polynucleotide encoding the above-mentioned CAR or dual CAR.

In another aspect, the application also provides the vector comprising the above-mentioned polynucleotide encoding the above-mentioned CAR or dual CAR.

In another aspect, the application also provides the composition comprising at least one of the above-mentioned nucleic acids or at least one of the above-mentioned vectors.

In another aspect, the application also provides a host cell comprising one or more nucleic acids or vectors or a composition of the present application.

In a further aspect, the host cell is an autologous cell or an allogeneic cell.

In a further aspect, the host cell is a mammalian cell, preferably a primate cell, more preferably a human cell.

In a further aspect, the host cell is selected from a T cell, a NK cell, an iNKT cell, a cord blood NK cell, a gamma delta T cell (γδ T-cell), a TCR knockout T cell, a virus-specific T cell, a monocyte, a macrophage, or an iPSC-derived T cell.

In another aspect, the application also provides a pharmaceutical composition comprising the CAR or dual CAR, one or more nucleic acids, one or more vectors, or a host cell of the present application.

In a further aspect, the pharmaceutical composition further comprises one or more pharmaceutically acceptable excipients.

In another aspect, the application relates to a method of treating disease in a subject in need thereof, comprising administering to the subject (e.g. a therapeutically effective amount of) the pharmaceutical composition, the CAR, the dual CAR of the present application.

In a further aspect, the disease is a cancer comprising hematological malignancy or one or more solid tumors.

In a further aspect, the cancer is ovarian cancer, pancreatic cancer, colon cancer, colorectal cancer, lymphoma, esophageal cancer, lung cancer, hepatic cancer, head-neck cancer, or cancer of the gallbladder.

In another aspect, the application relates to a method of reducing the cytotoxicity of a CAR-T cell towards normal cells, using the CAR or dual CAR, the nucleic acid, or the vector, or the composition of the present application.

In one aspect, the application relates to a method of producing a CAR-T cell with reduced cytotoxicity towards normal cells comprising:
(1) introducing to a host cell the nucleic acid, or the vector of the present application, and
(2) isolating and/or expanding the CAR-T cells following the introduction.

In a further aspect, the host cell is a mammalian cell, preferably a primate cell, more preferably a human cell.

In a further aspect, the host cell is selected from a T cell, a NK cell, an iNKT cell, a cord blood NK cell, a gamma delta T cell (γδ T-cell), a TCR knockout T cell, a virus-specific T cell, a monocyte, a macrophage, or an iPSC-derived T cell.

The technical solutions of the application have at least the following advantages:
1. The above-mentioned chimeric antigen receptor (CAR) is a target-dependent on-switch. In the absence of target antigen, CAR surface expression is undetectable. Due to its limited detectability in the absence of antigen, the present application refers to this system as a “stealth CAR”.
2. The stealth CAR can reduce cytotoxicity towards normal cells and improve CAR-T safety by using the transmembrane and juxtamembrane motifs of the IL2Rβ, LDLR, SEZ6L2, and/or degradation sequence (PSKFFSQL) of IL2R which results in greatly reduced CAR expression at the cell surface in the absence of antigen, while retaining downstream activation ability in response to antigen-expressing target cells.
3. The present application has shown that primary T cells expressing these surface-unstable stealth CARs are able to elicit antigen-dependent target cell killing. By limiting CAR activity in this way, the present application can reduce therapeutic toxicity and T cell exhaustion.

### BRIEF DESCRIPTION OF THE DRAWINGS

The novel features of the application are set forth with particularity in the appended claims.

Some of the features and advantages of the present application are explained in the following detailed description in the embodiments and in the examples.

FIG. 1. Cartoon representation of endocytic CAR system for addressing antigen escape. Selective pressure on the tumor cells through targeting of the primary tumor antigen via the primary CAR causes receptor downregulation and “antigen escape”. Surviving tumor cells can then be targeted via a secondary tumor antigen recognized by the secondary (endocytic) CAR.

FIG. 2. Cartoon representation of decreased CAR surface stability helps mitigate therapeutic toxicity. Tumor antigens are often protein markers that are upregulated on cancerous tissue but still expressed to a lower degree on normal tissue. Stabilization of the CAR on the T cell surface only in the presence of the corresponding antigen promotes killing of cells expressing larger amounts of the target protein.

FIG. 3. A schematic representation of endocytic CAR constructs. There are two variants of the CAR construct, one using the PSKFFSQL degradation sequence (DT) at the C-terminal of the CAR sequence and the other replacing the CD8 transmembrane domain with the IL2Rβ transmembrane domain and 27 amino acids juxtamembrane domain as well as an IL2Rβ degradation sequence (DT).

FIG. 4. Jurkat E6.1 NFAT-reporter cells show CAR expression post-electroporation. Jurkat cells were electroplated with PiggyBac Transposase mRNA and each variant of the CAR construct including MN14op CAR(LBC001), HA-MN14op CAR(MLB001), HA-MN14op CAR-DT(MLB002), and HA-MN14op CAR-IL2Rb-tm (MLB003). Receptor expression was observed using anti-human Fab′ (AF488). Dead cells were excluded by incorporation of DAPI.

FIG. 5. HA-tag and anti-human Fab (AF488) staining show linear correlation. Jurkat cells expressing the endocytic CAR variants (MLB002 and MLB003) were co-stained for HA (PE) and human Fab (AF488), showing a linear relationship between the two stains. Having an alternative staining strategy allows for the possibility of pulse-chase experiments to measure the rate of receptor internalization.

FIG. 6. Endocytic CAR expression in Primary T cells. Donor PBMC-derived T (Primary T cells) cells were electroporated with PiggyBac transposase mRNA and each of the endocytic CAR construct variants (MLB002 and MLB003). Receptor surface expression was evaluated by anti-human Fab′ (AF488) staining.

FIG. 7. Endocytic CARs (MLB002 and MLB003) drive downstream signaling in response to tumor antigen. Jurkat NFAT-luciferase reporter cells were co-cultured 1:1 with LOVO cells expressing the CEA target antigen. Jurkat cells incubated with LOVO cells showed luciferase activity, whereas Jurkat cells grown in the absence of target cells (LOVO negative) showed minimal reporter gene expression at 6-hours.

FIG. 8. Long-term stimulation shows comparable activity between CAR variants. Jurkat NFAT-luciferase reporter cells expressing the endocytic CAR constructs were co-cultured 1:1 with LOVO cells expressing CEA antigen. Following the 24-hour stimulation period, Jurkats expressing the IL2Rβ variants (MLB002 and MLB003) of the receptor showed comparable activation to the original MN14op-CAR.

FIG. 9. Endocytic CAR constructs direct target cell killing of Kato-III cells. Donor-derived primary T cells were incubated 1:1(5e5:5e5) with Kato-III luciferase reporter cells expressing the CEA antigen. After 24-hours, cytotoxicity was measured by luminescence indicating that both IL2Rβ variants of the CAR were able to direct antigen specific cytotoxicity above that of the untransduced control cells.

FIG. 10. A cartoon schematic of the CAR constructs (MLB047 and MLB048) illustrating alterations to the transmembrane and juxtamembrane sequences. MLB047 utilizes the MN14op CEA ScFv antigen recognition domain. MLB047 is designed with the Seizure-like 6 protein 2 (SEZ6L2) transmembrane and 33 amino acids from the juxtamembrane sequence. MLB048 utilizes the LDLR transmembrane and juxtamembrane sequences. The CARs do not include P2A-GFP.

FIG. 11. A cartoon schematic of the CAR constructs (MLB054 and MLB055) illustrating alterations to the transmembrane and juxtamembrane sequences. MLB054 utilizes the 841 Claudin 18.2 ScFv (CN02) antigen recognition domain. MLB054 is designed with the Seizure-like 6 protein 2 (SEZ6L2) transmembrane and 33 amino acids from the juxtamembrane sequence. MLB055 is a two-component CAR (dual CAR) based on the MLB054 Claudin18.2 CAR sequence and anti-PDL1 coupled to the CD28 signaling domain with the same CD8 hinge and transmembrane domains as the original CAR. The CARs do not include P2A-GFP.

FIG. 12. Expression profile of novel Stealth CAR constructs (MLB025, MLB020, MLB048 and MLB047) in model T cell line. MLB010 is a control CAR construct. Jurkat cells were transduced with PiggyBAC CAR constructs and PiggyBAC transposase mRNA. CAR expression was evaluated at day 10 post-electroporation by staining with anti-human F(ab′)₂ (AF647). Staining was performed at 4° C. for 30 minutes in serum-free staining media. GFP signal was used to evaluate construct expression in the absence of detectable surface staining.

FIG. 13. Stealth CAR constructs drive NFAT-dependent T cell activation. Jurkat Luciferase reporter cells transduced to express Stealth CAR constructs were co-cultured with CEA-positive LoVo cells for 24 hours and T cell activation was evaluated by NFAT-driven luciferase reporter expression. 25,000 CAR-positive Jurkat reporter cells were cultured 1:1 with the target cell for this assay. The number of CAR-positive Jurkat cells was determined by co-expression of GFP on the CAR vector.

FIG. 14A-F. Stealth CAR formats show weaker activation profile relative to CD8-based CAR format. Jurkat T cells expressing MLB010, MLB020, MLB025, MLB048, and MLB047 were co-cultured with LoVo cells for 3 hours, 6 hours or 24 hours and Jurkat cell activation was measured by CD69 expression detected by flow cytometry using a PE conjugated anti-CD69 antibody. CAR positive cells were detected based on GFP expression, while T cells were identified by expression of CD3 to delineate the transduced and untransduced populations. Shown here are the scatter plots and corresponding histograms of CD69 expression. FIG. 14A shows the CAR positive population result of MLB010(M10) and MLB020(M20). FIG. 14B shows the CAR positive population result of MLB025(M25) and MLB048(M48). FIG. 14C shows the CAR positive population result of MLB047(M47) and non-transduced (UTD). FIG. 14D shows the overall degree of CD69 upregulation result of MLB010(M10) and MLB020(M20). FIG. 14E shows the overall degree of CD69 upregulation result of MLB025(M25) and MLB048(M48). FIG. 14F shows the overall degree of CD69 upregulation result of MLB047(M47) and non-transduced (UTD).

FIG. 15. Quantification of Jurkat activation in Stealth CARs shows reduced activation state following co-culture with antigen-expressing cells. The histograms in FIG. 15 were quantified to obtain the fraction of activated cells and the extent of CD69 expression in the CAR T cell populations. We observed comparable activation kinetics in response to antigen as seen in the fraction of activated cells, however the Stealth CARs MLB020 (M20), MLB025 (M25), MLB048 (M48), MLB047 (M47) showed much lower CD69 expression relative to the original MLB010 (M10) CAR format.

FIG. 16. Expression profile of novel Stealth CAR constructs (MLB020, MLB047, MLB048) in primary cells. Primary human PBMC-derived T cells were transduced with PiggyBAC CAR constructs and PiggyBAC transposase mRNA. CAR expression was evaluated at day 10 post-electroporation by staining with anti-human F(ab′)₂ (AF647). Staining was performed at 4° C. for 30 minutes in serum-free staining media. GFP signal was used to evaluate construct expression in the absence of detectable surface staining.

FIG. 17A-C. Stealth CARs (MLB013, MLB048, MLB047) drive variable cell-mediated cytotoxicity dependent on target cell antigen expression. Three representative CEA-positive target cell lines: LoVo (CEA^{Hi}) (FIG. 17A), A549 (CEA^{Mid}) (FIG. 17B), and HT29 (CEA^{Lo}) (FIG. 17C) were used to assay Stealth CAR-T cell cytotoxic potential. T cells were cultured with target cells at ratios of 3:1, 1:1, and 0.3:1 (CAR+ T cells:Target), where the number of target cells per well was fixed at 10,000. Target cells expressed a constitutive luciferase reporter and cytotoxicity was evaluated as a decrease in luminescent signal relative to wells containing only the target cell line. Percent cytotoxicity was calculated based on the T cell-negative control (E:T=0:1) and T cell only control (E:T=1:0).

FIG. 18A-C. Stealth CAR (MLB013, MLB048, MLB047) T-cells show dramatically reduced inflammatory cytokine expression profile. Supernatant from cytotoxicity assays against three representative CEA-positive target cell lines: LoVo (CEA^{Hi}) (FIG. 18A), A549 (CEA^{Mid}) (FIG. 18B), and HT29 (CEA^{Lo}) (FIG. 18C) was harvested and IFNγ expression was determined by ELISA. T cells were cultured with target cells at ratios of 3:1, 1:1, and 0.3:1 (CAR+ T cells:Target), where the number of target cells per well was fixed at 10,000. Target cells expressed a constitutive luciferase reporter and cytotoxicity was evaluated as a decrease in luminescent signal relative to wells containing only the target cell line. Percent cytotoxicity was calculated based on the T cell-negative control (E:T=0:1) and T cell only control (E:T=1:0).

FIG. 19. Stealth CAR (MLB013, MLB048, MLB047) T-cells show dramatically reduced IL-2 expression profile. Supernatant from cytotoxicity assays against two representative CEA-positive target cell lines: A549 (CEA^{Mid}) and HT29 (CEA^{Lo}) was harvested and IL-2 expression was determined by ELISA. T cells were cultured with target cells at ratios of 3:1, 1:1, and 0.3:1 (CAR+ T cells:Target), where the number of target cells per well was fixed at 10,000. Target cells expressed a constitutive luciferase reporter and cytotoxicity was evaluated as a decrease in luminescent signal relative to wells containing only the target cell line. Percent cytotoxicity was calculated based on the T cell-negative control (E:T=0:1) and T cell only control (E:T=1:0).

FIG. 20. Expression of Claudin18.2-specific Stealth CAR (MLB026, MLB054, MLB055) T cells in Jurkat T cell model system. Jurkat cells were transduced with PiggyBAC CAR constructs for LBC010, MLB026, MLB054, and MLB055 and PiggyBAC transposase mRNA. CAR expression was evaluated at day 10 post-electroporation by staining with anti-human F(ab′)₂ (AF647). Staining was performed at 4° C. for 30 minutes in serum-free staining media. GFP signal was used to evaluate construct expression in the absence of detectable surface staining. LBC010 and MLB026 have the same CAR sequence, but MLB026 also has joint expression of GFP on the CAR construct. Detection of scFv at the surface in MLB055 is due to the presence of an anti-PD-L1 domain in the joint expression construct.

FIG. 21A-D. Claudin18.2 stealth CAR variants show antigen-specific activation. Jurkat T cells expressing MLB026, MLB054, and MLB055 were co-cultured overnight with HEK293T, HEK293T-cldn18.2, or NUGC4-cldn18.2 cells and activation was measured by flow cytometry using a PE-conjugated anti-CD19 antibody. T cells were identified by CD3 expression. We saw increased tonic signaling in the CD8 CAR format as evidenced by the upregulation of CD69 in response to the negative cell line. Both MLB054 and MLB055 showed reduced baseline activation and antigen-specific upregulation of CD69. FIG. 21A-D show that both endocytic (MLB054, MLB055) and non-endocytic CAR (MLB026) have CD69 upregulation. FIG. 21A-B show CAR positive population, and FIG. 21C-D show the overall degree of CD69 upregulation.

FIG. 22. Quantification of CD69 expression shows reduced activation of stealth CAR variants. Quantification of the CD69 expression in FIG. 22 showed the CAR variants MLB054 and MLB055 had reduced CD69 expression as well as fewer responding cells in response to claudin18.2-expressing target cells, although both MLB054 and MLB055 showed reduced baseline activation relative to MLB026. The left of FIG. 22 represents CD69 expression, and the right of FIG. 22 represent CD69+%.

FIG. 23. Donor-derived T cells were transduced via electroporation with constructs MLB026 and MLB054 and CAR expression was evaluated at day 7 post-electroporation. Cells were stained with AF647-conjugated anti-human F(ab′)₂ antibody.

FIG. 24. Donor-derived T cells expressing MLB026 or MLB054 were co-cultured with HEKcldn18.2 Luciferase-expressing cells for 24 hours after which luminescence was quantified by the addition of an equal volume of NeoLite substrate. Percent cytotoxicity was evaluated as a decrease relative to the untreated control cells.

FIG. 25. Donor-derived T cells expressing MLB026 or MLB054 were co-cultured with NUGC4cldn18.2 Luciferase-expressing cells for 24 hours after which luminescence was quantified by the addition of an equal volume of NeoLite substrate. Percent cytotoxicity was evaluated as a decrease relative to the untreated control cells.

FIG. 26. Supernatant was harvested from the 24-hour cytotoxicity assay for MLB026 and MLB054 in coculture with HEKcldn18.2 Luciferase-expressing cells and evaluated for IFN-γ expression by ELISA.

FIG. 27. Supernatant was harvested from the 24-hour cytotoxicity assay for MLB026 and MLB054 in coculture with NUGC4cldn18.2 Luciferase-expressing cells and evaluated for IFN-7 expression by ELISA.

FIG. 28A-B. Donor-derived T cells were transduced via electroporation with constructs MLB038, MLB039, MLB079, and MLB080 and CAR expression was evaluated at day 7 post-electroporation. Cells were stained with AF647-conjugated anti-human F(ab′)2 antibody. FIG. 28A is the result of UTD, MLB038, and MLB039. FIG. 28B is the result of MLB079 and MLB080.

FIG. 29. MDA-MB-231 and SK-BR-3 cells were stained with either fluorescently-conjugated anti-human HER2 or a non-specific control antibody. LoVo cells were stained with fluorescently-conjugated anti-human HER2. All stained cells were compared to an unstained control.

FIG. 30. Donor-derived T cells expressing MLB038, MLB039, MLB079, or MLB080 were co-cultured with MDA-MB-231 Luciferase-expressing cells for 24 hours after which luminescence was quantified by the addition of an equal volume of NeoLite substrate. Percent cytotoxicity was evaluated as a decrease relative to the untreated control cells.

FIG. 31. Donor-derived T cells expressing MLB038, MLB039, MLB079, or MLB080 were co-cultured with LoVo Luciferase-expressing cells for 24 hours after which luminescence was quantified by the addition of an equal volume of NeoLite substrate. Percent cytotoxicity was evaluated as a decrease relative to the untreated control cells.

FIG. 32. Donor-derived T cells expressing MLB038, MLB039, MLB079, or MLB080 were co-cultured with SK-BR-3 Luciferase-expressing cells for 24 hours after which luminescence was quantified by the addition of an equal volume of NeoLite substrate. Percent cytotoxicity was evaluated as a decrease relative to the untreated control cells.

FIG. 33. Supernatant was harvested from the 24 hour cytotoxicity assay for MLB038, MLB039, MLB079, or MLB080 in coculture with MDA-MB-231 Luciferase-expressing cells and evaluated for IFN-γ expression by ELISA.

FIG. 34. Supernatant was harvested from the 24 hour cytotoxicity assay for MLB038, MLB039, MLB079, or MLB080 in coculture with LoVo Luciferase-expressing cells and evaluated for IFN-γ expression by ELISA.

FIG. 35. Supernatant was harvested from the 24 hour cytotoxicity assay for MLB038, MLB039, MLB079, or MLB080 in coculture with SK-BR-3 Luciferase-expressing cells and evaluated for IFN-γ expression by ELISA.

FIG. 36. HEKcldn18.2 Luciferase-expressing cells were stained for anti-human HER2 or Claudin18.2. Claudin18.2 was detected by both the 841 clone and the 808 clone. Stronger staining for claudin18.2 was observed when using the 808 clone, although claudin18.2 was still detectable by 841. When staining for claudin18.2, cells were first stained with the unconjugated humanized 841 and 808 antibodies and binding was detected using a fluorescently-conjugated, murine anti-human IgG secondary antibody.

FIG. 37. A schematic overview of the claudin18.2/HER2 dual CAR constructs in comparison to the MLB026 claudin18.2 single CAR control. MLB108 was engineered with a dominant claudin18.2 CAR identical to MLB026 and a second, non-dominant CAR based on the Herceptin anti-HER2 antibody domain, the SEZ6L2 transmembrane and juxtamembrane domain, and the CD3ζ ITAM signaling motifs. The MLB040 dual CAR was engineered with a dominant claudin18.2 CAR identical to MLB026 and a second, non-dominant CAR based the Herceptin anti-HER2 antibody domain, the transmembrane and juxtamembrane sequences of IL2Rβ, the CD28 coreceptor signaling domain, and the CD3ζ intracellular ITAM signaling motifs.

FIG. 38. Donor-derived T cells were transduced via electroporation with constructs MLB026 and MLB108 and CAR expression was evaluated at day 7 post-electroporation. Cells were stained with AF594-conjugated anti-human F(ab′)₂ antibody.

FIG. 39. Donor-derived T cells expressing MLB026 and MLB108 were co-cultured with HEKcldn18.2 Luciferase-expressing cells for 24 hours after which luminescence was quantified by the addition of an equal volume of NeoLite substrate. Percent cytotoxicity was evaluated as a decrease relative to the untreated control cells.

FIG. 40. Supernatant was harvested from the 24 hours cytotoxicity assay for MLB026 and MLB108 in coculture with HEKcldn18.2 Luciferase-expressing cells and evaluated for IFN-γ expression by ELISA.

FIG. 41A-B. Donor-derived T cells were transduced via electroporation with constructs MLB026, MLB038, MLB039, and MLB040 and CAR expression was evaluated at day 7 post-electroporation. Cells were stained with AF647-conjugated anti-human F(ab′)₂ antibody. FIG. 41A is the result of UTD, MLB026, and MLB038. FIG. 41B is the result of MLB039 and MLB040.

FIG. 42. Donor-derived T cells expressing MLB026, MLB038, MLB039 or MLB040 were co-cultured with HEKcldn18.2 Luciferase-expressing cells for 24 hours after which luminescence was quantified by the addition of an equal volume of NeoLite substrate. Percent cytotoxicity was evaluated as a decrease relative to the untreated control cells.

FIG. 43. Supernatant was harvested from the 24 hours cytotoxicity assay for MLB026, MLB038, MLB039, and MLB040 in coculture with HEKcldn18.2 Luciferase-expressing cells and evaluated for IFN-γ expression by ELISA.

FIG. 44. Donor-derived T cells expressing MLB026, MLB038, MLB039 or MLB040 were co-cultured with SK-BR-3 Luciferase-expressing cells for 24 hours after which luminescence was quantified by the addition of an equal volume of NeoLite substrate. Percent cytotoxicity was evaluated as a decrease relative to the untreated control cells.

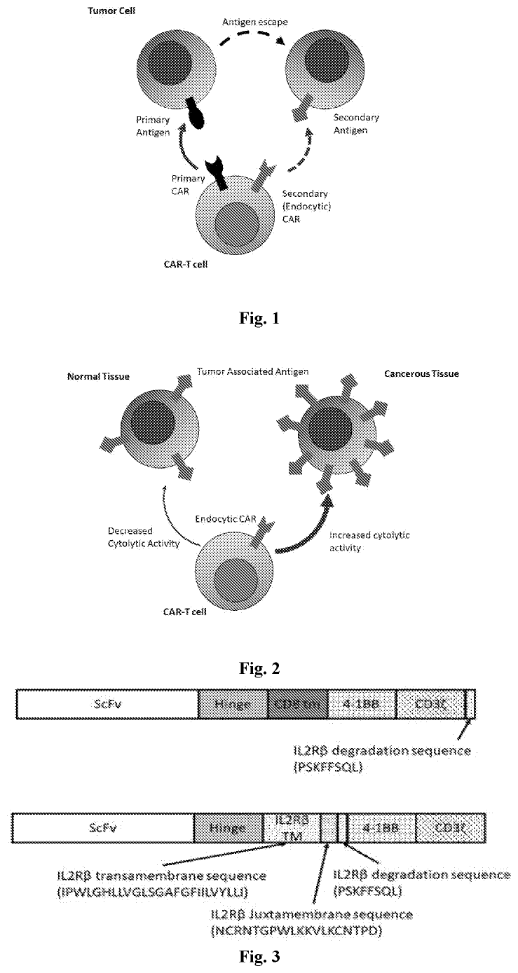

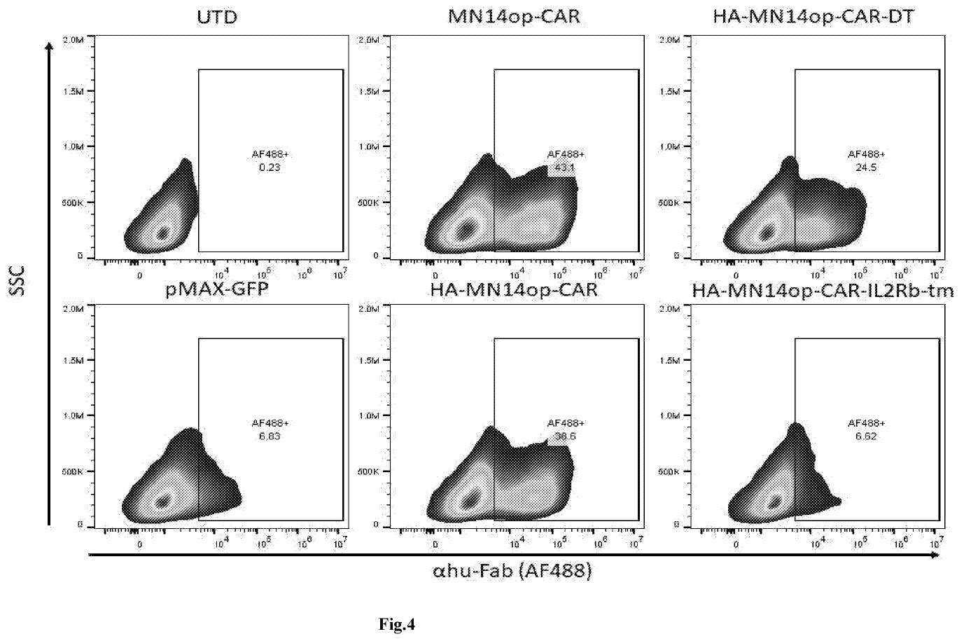

### DETAILED DESCRIPTION OF THE INVENTION

Unless defined otherwise, technical and scientific terms used herein have the same meaning as generally used in the art to which this disclosure belongs. For purposes of interpreting this specification, the following definitions will apply and whenever appropriate, terms used in the singular will also include the plural and vice versa. As used herein and in the appended claims, the singular forms “a”, “an”, and “the” also refer to the plural forms unless the context clearly dictates otherwise, e.g., reference to “a host cell” includes a plurality of such host cells.

As used herein, the term a “chimeric antigen receptor (CAR)” means a fused protein comprising an extracellular domain capable of binding to a predetermined antigen, an intracellular domain comprising a signaling domain, and a transmembrane domain. The phrase “binding to a predetermined antigen” means any proteinaceous molecule or part thereof that can specifically bind to the predetermined antigen. The “signaling domain” means any oligopeptide or polypeptide domain known to function to transmit a signal causing activation or inhibition of a biological process in a cell, for example, activation of an immune cell such as a T cell or a NK cell. Examples include 4-1BB, CD28 and/or CD3ζ signaling domain.

As used herein, the term a “IL2R” or “IL2Rb” means the IL2 Receptor β chain. The high-affinity IL2 Receptor is a transmembrane receptor comprised of three distinct, noncovalently associated components: the α, β, and γ chains. It has been shown that in the absence of the α and γ subunits, the β chain is constitutively endocytosed and degraded.

As used herein, the term a “Stealth CAR” or “Endocytic CAR” refers in its broadest sense to a chimeric antigen receptor (CAR) comprising: (1) an extracellular ligand-binding domain comprising a single chain variable fragment (scFv) specifically binding to a predetermined antigen; (2) a transmembrane (tm) linking juxtamembrane (jm) domain; wherein the transmembrane linking juxtamembrane domain comprises an IL2 Receptor 3 chain (IL2Rβ) transmembrane domain and an IL2Rβ juxtamembrane domain, and the transmembrane linking juxtamembrane domain is adjacent to IL2Rβ degradation sequence (DT); wherein preferably, the IL2R degradation sequence is at the C-terminal of the transmembrane linking juxtamembrane domain; wherein preferably, the IL2R degradation sequence comprises an amino acid sequence that is at least about 95%, 96%, 97%, 98%, 99% or 100% identical to the amino acid sequence represented by SEQ ID NO: 2; wherein the transmembrane linking juxtamembrane domain comprises a Low-Density Lipoprotein Receptor (LDLR) transmembrane domain and an LDLR juxtamembrane domain; or wherein the transmembrane linking juxtamembrane domain comprises a Seizure 6-like Protein 2 (SEZ6L2) transmembrane domain and a SEZ6L2 juxtamembrane domain; (3) an intracellular domain; wherein preferably, the intracellular domain comprises a signaling domain; more preferably, the signaling domain comprises one or more signaling domains selected from the group consisting of a 4-1BB signaling domain, a CD28 signaling domain and a CD3ζ signaling domain. Or a chimeric antigen receptor (CAR) comprising: (1) an extracellular ligand-binding domain comprising single chain variable fragment (scFv) specifically binding to a predetermined antigen; (2) a transmembrane domain, and (3) a cytoplasmic segment comprising an IL2Rβ degradation sequence (DT) and at least one signaling domain; wherein preferably, the IL2Rβ degradation sequence comprises an amino acid sequence that is at least about 95%, 96%, 97%, 98%, 99% or 100% identical to the amino acid sequence represented by SEQ ID NO: 2; wherein preferably, the IL2Rβ degradation sequence is at the C-terminal of the cytoplasmic segment. Or a dual CAR comprising a first CAR according to any of the above-described embodiments, and a second CAR.

As used herein, the term an “antigen binding fragment” or “antigen binding molecule” refers in its broadest sense to a molecule that specifically binds an antigenic determinant. Examples of antigen binding molecules are antibodies, antibody fragments and scaffold antigen binding proteins. The term “antibody” herein is used in the broadest sense and encompasses various antibody structures, including but not limited to monoclonal antibodies, polyclonal antibodies, monospecific and multispecific antibodies (e.g., bispecific antibodies), and antibody fragments so long as they exhibit the desired antigen-binding activity.

As used herein, the term a “single-chain variable fragment (scFv)” is a fusion protein of the variable regions of the heavy (VH) and light chains (VL) of an antibody, connected with a short linker peptide of ten to about 25 amino acids. The linker is usually rich in glycine for flexibility, as well as serine or threonine for solubility, and can either connect the N-terminus of the VH with the C-terminus of the VL, or vice versa. This protein retains the specificity of the original antibody, despite removal of the constant regions and the introduction of the linker. In addition, antibody fragments comprising single chain polypeptides have the characteristics of a VH domain, namely being able to assemble together with a VL domain, or of a VL domain, namely being able to assemble together with a VH domain to a functional antigen binding site, thereby providing the antigen binding properties of full length antibodies.

As used herein, the term a “therapeutically effective amount” of an agent, e.g., a pharmaceutical composition, refers to an amount effective, at dosages and for periods of time necessary, to achieve the desired therapeutic or prophylactic result. A therapeutically effective amount of an agent for example eliminates, decreases, delays, minimizes or prevents adverse effects of a disease.

As used herein, the term an “individual” or “subject” is a mammal. Mammals include, but are not limited to, domesticated animals (e.g., cows, sheep, cats, dogs, and horses), primates (e.g., humans and non-human primates such as monkeys), rabbits, and rodents (e.g., mice and rats). Particularly, the individual or subject is a human. The term “pharmaceutical composition” refers to a preparation which is in such form as to permit the biological activity of an active ingredient contained therein to be effective, and which contains no additional components which are unacceptably toxic to a subject to which the formulation would be administered. A “pharmaceutically acceptable excipient” refers to an ingredient in a pharmaceutical composition, other than an active ingredient, which is nontoxic to a subject. A pharmaceutically acceptable excipient includes, but is not limited to, a buffer, a stabilizer, or a preservative.

As used herein, “treatment” (and grammatical variations thereof such as “treat” or “treating”) refers to clinical intervention in an attempt to alter the natural course of the individual being treated, and can be performed either for prophylaxis or during the course of clinical pathology. Desirable effects of treatment include, but are not limited to, preventing occurrence or recurrence of disease, alleviation of symptoms, diminishment of any direct or indirect pathological consequences of the disease, preventing metastasis, decreasing the rate of disease progression, amelioration or palliation of the disease state, and remission or improved prognosis. In some embodiments, the molecules of the application are used to delay development of a disease or to slow the progression of a disease.

As used herein, the term “tumor-associated antigens (TAAs)” or “tumor antigens” means a biological molecule having antigenicity, the expression of which comes to be recognized in association with malignant alteration of a cell. The tumor antigen in the present disclosure includes a tumor specific antigen (an antigen which is present only in tumor cells and is not found in other normal cells), and a tumor-associated antigen (an antigen which is also present in other organs and tissues or heterogeneous and allogeneic normal cells, or an antigen which is expressed during development and/or differentiation).

The term “amino acid” as used within this application denotes the group of naturally occurring carboxy α-amino acids comprising alanine (three letter code: Ala, one letter code: A), arginine (Arg, R), asparagine (Asn, N), aspartic acid (Asp, D), cysteine (Cys, C), glutamine (Gln, Q), glutamic acid (Glu, E), glycine (Gly, G), histidine (His, H), isoleucine (Ile, I), leucine (Leu, L), lysine (Lys, K), methionine (Met, M), phenylalanine (Phe, F), proline (Pro, P), serine (Ser, S), threonine (Thr, T), tryptophan (Trp, W), tyrosine (Tyr, Y), and valine (Val, V). “Percent (%) amino acid sequence identity” with respect to a reference polypeptide (protein) sequence is defined as the percentage of amino acid residues in a candidate sequence that are identical with the amino acid residues in the reference polypeptide sequence, after aligning the sequences and introducing gaps, if necessary, to achieve the maximum percent sequence identity, and not considering any conservative substitutions as part of the sequence identity. Alignment for purposes of determining percent amino acid sequence identity can be achieved in various ways that are within the skill in the art, for instance, using publicly available computer software such as BLAST, BLAST-2, ALIGN. SAWI or Megalign (DNASTAR) software. Those skilled in the art can determine appropriate parameters for aligning sequences, including any algorithms needed to achieve maximal alignment over the full length of the sequences being compared. For purposes herein, however, % amino acid sequence identity values are generated using the sequence comparison computer program ALIGN-2. The ALIGN-2 sequence comparison computer program was authored by Genentech, Inc., and the source code has been filed with user documentation in the U.S. Copyright Office, Washington D.C., 20559, where it is registered under U.S. Copyright Registration No. TXU510087. The ALIGN-2 program is publicly available from Genentech, Inc., South San Francisco, Calif., or may be compiled from the source code. The ALIGN-2 program should be compiled for use on a UNIX operating system, including digital UNIX V4.0D. All sequence comparison parameters are set by the ALIGN-2 program and do not vary.

The terms “host cell”, “host cell line,” and “host cell culture” are used interchangeably and refer to cells into which exogenous nucleic acid has been introduced, including the progeny of such cells. Host cells include “transformants” and “transformed cells,” which include the primary transformed cell and progeny derived therefrom without regard to the number of passages.

The term “cancer” or “tumor” as used herein refers to proliferative diseases, such as ovarian cancer, pancreatic cancer, colon cancer, colorectal cancer, lymphomas, lymphocytic leukemias, lung cancer, non-small cell lung (NSCL) cancer, bronchioloalviolar cell lung cancer, bone cancer, pancreatic cancer, skin cancer, cancer of the head or neck, cutaneous or intraocular melanoma, uterine cancer, ovarian cancer, rectal cancer, cancer of the anal region, stomach cancer, gastric cancer, breast cancer, uterine cancer, carcinoma of the fallopian tubes, carcinoma of the endometrium, carcinoma of the cervix, carcinoma of the vagina, carcinoma of the vulva, Hodgkin's Disease, cancer of the esophagus, cancer of the small intestine, cancer of the endocrine system, cancer of the thyroid gland, cancer of the parathyroid gland, cancer of the adrenal gland, sarcoma of soft tissue, cancer of the urethra, cancer of the penis, prostate cancer, cancer of the bladder, cancer of the kidney or ureter, renal cell carcinoma, carcinoma of the renal pelvis, mesothelioma, hepatocellular cancer, biliary cancer, neoplasms of the central nervous system (CNS), spinal axis tumors, brain stem glioma, glioblastoma multiforme, astrocytomas, schwanomas, ependymonas, medulloblastomas, meningiomas, squamous cell carcinomas, pituitary adenoma and Ewings sarcoma, including refractory versions of any of the above cancers, or a combination of one or more of the above cancers.

### EXAMPLES

### Example 1. Design and Generation of Endocytic CAR Constructs (MLB003 and MLB002)

Preparation of CAR constructs is a common technical method in the art. For example, first, CAR gene fragments are prepared through gene synthesis technology, then CAR PiggyBac transposon expression vectors are constructed, CAR constructs are electroporated into a target cell, and construct expression is assessed by flow cytometry or total protein analysis.

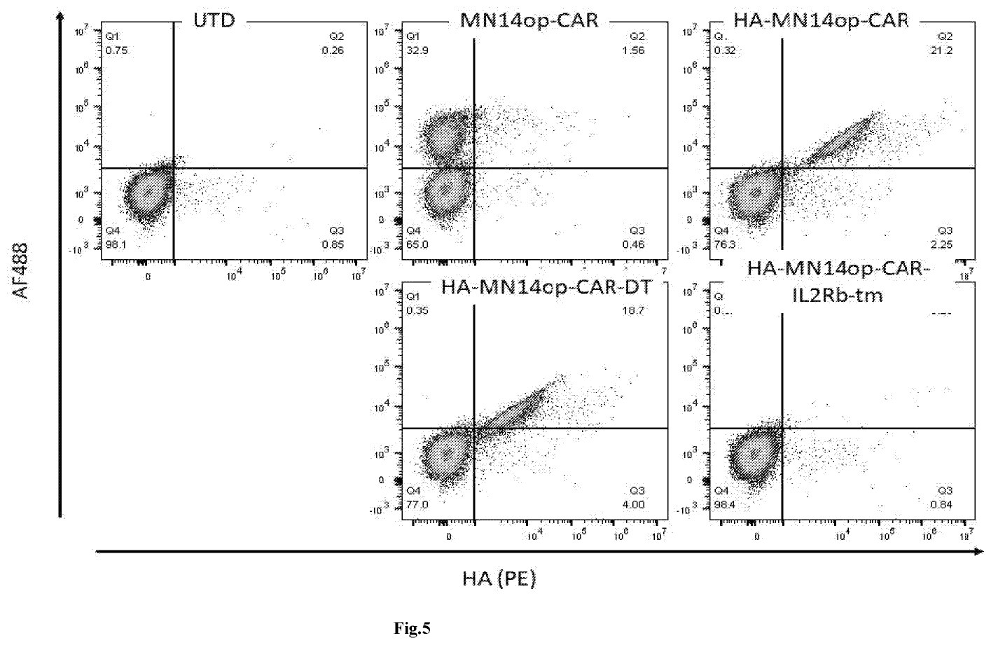

In this Example 1, two novel CAR constructs based on the IL2Rβ chain transmembrane and juxtamembrane motifs were designed (FIG. 3).
(1) CAR construct containing cytoplasmic segment comprising an IL2Rβ degradation sequence (DT):
HA-MN14op-CAR-DT (MLB002: HA-MN14ops cFv-CD8Hinge-CD8 tm-4-1BB-CD3ζ-DT, SEQ ID NO:8);
(2) CAR construct containing a IL2Rβ transmembrane domain, a IL2Rβ juxtamembrane domain, and further containing a IL2Rβ degradation sequence (DT):
HA-MN14op-CAR-IL2Rb-tm (MLB003: HA-MN14op CEA scFv-CD8Hinge-IL2Rβ tm jm DT-4-1BB-CD3ζ, SEQ ID NO:9)
(3) The original CAR construct using a CD8 transmembrane domain: MN14op-CAR (LBC001: MN14op CEA scFv-CD8Hinge-CD8tm-4-1BB-CD3).
(4) The control CAR construct using a CD8 transmembrane domain and HA sequence.

HA-MN14op CAR (MLB001: HA-MN14op CEA scFv-CD8Hinge-CD8tm-4-1BB-CD3).

The pMAX-GFP is a control plasmid used to determine electroporation efficiency, particularly when a fluorescent marker is not available in the test construct.

Functional efficacy was evaluated by T cell activation assays, including but not limited to NFAT reporter assays and cytotoxicity assays.

TABLE 1
The sequences used in Examples 1-5

| DESCRIPTION | | SEQ |
|---|---|---|
| (CAR construct) | SEQUENCE | ID NO: |
| IL2Rβ | IPWLGHLLVGLSGAFGFIILVYLLI | 1 |
| transmembrane | | |
| domain (tm) | | |
| IL2Rβ degradation | PSKFFSQL | 2 |
| sequence (DT) | | |
| IL2Rβ truncated | NCRNTGPWLKKVLKCNTPDPSKFFSQL | 3 |
| cytoplasmic domain | | |
| IL2Rβ | NCRNTGPWLKKVLKCNTPD | 4 |
| juxtamembrane | | |
| sequence (jm) | | |
| MN14op CEA scFv | EVQLVESGGGVVKPGGSLRLSCSASGFDFTTYWMSWVRQAPGKG | 5 |
| | LEWIGEIHPDSSTINYAPSLKDRFTISRDNAKNTLYLQMDSLRPEDT | |
| | GVYYCASLYFGFPWFAYWGQGTTVTVSSGGGGSGGGGSGGGGS | |
| | GGGGSDIVLTQSPSSLSASLGDRVTITCKASQDVGTSVAWYQQKP | |
| | GKAPKLLIYWTSTRHTGVPSRFSGSGSGTDFTFTISSLQPEDIATYY | |
| | CQQYSLYRSFGGGTKVEIKG | |
| MN14op CAR | MALPVTALLLPLALLLHAARPEVQLVESGGGVVKPGGSLRLSCSA | 6 |
| (LBC001) | SGFDFTTYWMSWVRQAPGKGLEWIGEIHPDSSTINYAPSLKDRFTI | |
| (MN14op CEA scFv- | SRDNAKNTLYLQMDSLRPEDTGVYYCASLYFGFPWFAYWGQGT | |
| CD8Hinge-CD8tm-4- | TVTVSSGGGGSGGGGSGGGGSGGGGSDIVLTQSPSSLSASLGDRV | |
| 1BB-CD3ζ) | TITCKASQDVGTSVAWYQQKPGKAPKLLIYWTSTRHTGVPSRFSG | |
| | SGSGTDFTFTISSLQPEDIATYYCQQYSLYRSFGGGTKVEIKGTTTP | |
| | APRPPTPAPTIASQPLSLRPEACRPAAGGAVHTRGLDFACDIYIWAPL | |
| | AGTCGVLLLSLVITLYCKRGRKKLLYIFKQPFMRPVQTTQEEDGC | |
| | SCRFPEEEEGGCELRVKFSRSADAPAYKQGQNQLYNELNLGRREE | |
| | YDVLDKRRGRDPEMGGKPRRKNPQEGLYNELQKDKMAEAYSEI | |
| | GMKGERRRGKGHDGLYQGLSTATKDTYDALHMQALPPR* | |
| HA-MN14op CAR | MALPVTALLLPLALLLHAARPYPYDVPDYAEVQLVESGGGVVKPG | 7 |
| (MLB001) | GSLRLSCSASGFDFTTYWMSWVRQAPGKGLEWIGEIHPDSSTINY | |
| (HA-MN14op CEA | APSLKDRFTISRDNAKNTLYLQMDSLRPEDTGVYYCASLYFGFPW | |
| scFv-CD8Hinge- | FAYWGQGTTVTVSSGGGGSGGGGSGGGGSGGGGSDIVLTQSPSS | |
| CD8tm-4-1BB- | LSASLGDRVTITCKASQDVGTSVAWYQQKPGKAPKLLIYWTSTRH | |
| CD3ζ) | TGVPSRFSGSGSGTDFTFTISSLQPEDIATYYCQQYSLYRSFGGGTK | |
| | VEIKGTTTPAPRPPTPAPTIASQPLSLRPEACRPAAGGAVHTRGLDFAC | |
| | DIYIWAPLAGTCGVLLLSLVITLYCKRGRKKLLYIFKQPFMRPVQT | |
| | TQEEDGCSCRFPEEEEGGCELRVKFSRSADAPAYKQGQNQLYNEL | |
| | NLGRREEYDVLDKRRGRDPEMGGKPRRKNPQEGLYNELQKDKM | |
| | AEAYSEIGMKGERRRGKGHDGLYQGLSTATKDTYDALHMQALPP | |
| | R* | |
| HA-MN14op CAR- | MALPVTALLLPLALLLHAARPYPYDVPDYAEVQLVESGGGVVKPG | 8 |
| DT | GSLRLSCSASGFDFTTYWMSWVRQAPGKGLEWIGEIHPDSSTINY | |
| (HA-MN14op scFv- | APSLKDRFTISRDNAKNTLYLQMDSLRPEDTGVYYCASLYFGFPW | |
| CD8Hinge-CD8 tm- | FAYWGQGTTVTVSSGGGGSGGGGSGGGGSGGGGSDIVLTQSPSS | |
| 4-1BB-CD3ζ-DT) | LSASLGDRVTITCKASQDVGTSVAWYQQKPGKAPKLLIYWTSTRH | |
| (MLB002) | TGVPSRFSGSGSGTDFTFTISSLQPEDIATYYCQQYSLYRSFGGGTK | |
| | VEIKGTTTPAPRPPTPAPTIASQPLSLRPEACRPAAGGAVHTRGLDFAC | |
| | DIYIWAPLAGTCGVLLLSLVITLYCKRGRKKLLYIFKQPFMRPVQT | |
| | TQEEDGCSCRFPEEEEGGCELRVKFSRSADAPAYKQGQNQLYNEL | |
| | NLGRREEYDVLDKRRGRDPEMGGKPRRKNPQEGLYNELQKDKM | |
| | AEAYSEIGMKGERRRGKGHDGLYQGLSTATKDTYDALHMQALPP | |
| | RPSKFFSQL* | |
| HA-MN14op CAR- | MALPVTALLLPLALLLHAARPYPYDVPDYAEVQLVESGGGVVKPG | 9 |
| IL2Rb-tm | GSLRLSCSASGFDFTTYWMSWVRQAPGKGLEWIGEIHPDSSTINY | |
| (HA-MN14op CEA | APSLKDRFTISRDNAKNTLYLQMDSLRPEDTGVYYCASLYFGFPW | |
| scFv-CD8Hinge- | FAYWGQGTTVTVSSGGGGSGGGGSGGGGSGGGGSDIVLTQSPSS | |
| IL2Rβ tm jm DT-4- | LSASLGDRVTITCKASQDVGTSVAWYQQKPGKAPKLLIYWTSTRH | |
| 1BB-CD3ζ) | TGVPSRFSGSGSGTDFTFTISSLQPEDIATYYCQQYSLYRSFGGGTK | |
| (MLB003) | VEIKGTTTPAPRPPTPAPTIASQPLSLRPEACRPAAGGAVHTRGLDFAC | |
| | DIPWLGHLLVGLSGAFGFIILVYLLINCRNTGPWLKKVLKCNT | |
| | PDPSKFFSQLKRGRKKLLYIFKQPFMRPVQTTQEEDGCSCRFPEEE | |
| | EGGCELRVKFSRSADAPAYKQGQNQLYNELNLGRREEYDVLDKR | |
| | RGRDPEMGGKPRRKNPQEGLYNELQKDKMAEAYSEIGMKGERR | |
| | RGKGHDGLYQGLSTATKDTYDALHMQALPPR* | |
| CAR-DTAscFv | TTTPAPRPPTPAPTIASQPLSLRPEACRPAAGGAVHTRGLDFACDIYIW | 10 |
| | APLAGTCGVLLLSLVITLYCKRGRKKLLYIFKQPFMRPVQTTQEED | |
| | GCSCRFPEEEEGGCELRVKFSRSADAPAYKQGQNQLYNELNLGRR | |
| | EEYDVLDKRRGRDPEMGGKPRRKNPQEGLYNELQKDKMAEAYS | |
| | EIGMKGERRRGKGHDGLYQGLSTATKDTYDALHMQALPPRPSKF | |
| | FSQL* | |
| CAR- IL2Rβ TM Δ | TTTPAPRPPTPAPTIASQPLSLRPEACRPAAGGAVHTRGLDFACDIPW | 11 |
| scFv | LGHLLVGLSGAFGFIILVYLLINCRNTGPWLKKVLKCNTPDPS | |
| | KFFSQLKRGRKKLLYIFKQPFMRPVQTTQEEDGCSCRFPEEEEGG | |
| | CELRVKFSRSADAPAYKQGQNQLYNELNLGRREEYDVLDKRRGR | |
| | DPEMGGKPRRKNPQEGLYNELQKDKMAEAYSEIGMKGERRRGK | |
| | GHDGLYQGLSTATKDTYDALHMQALPPR* | |

*Leader sequence is underlined, HA tag is italicized, IL2Rβ sequences are bold, CD8 Hinge is underlined and italicized.

### Example 2. Delivery of CAR Constructs Using Transposon for the Modification of Primary Human T Cells and Jurkat E6.1 Cells

Transposon-mediated delivery of endocytic CAR constructs in Example 1: The EF1α promoter was used to drive expression of our CAR constructs in a transposon vector.

Transduction of primary T cells and Jurkat E6.1 T cells (Jurkat E6.1 cells) with CAR constructs: PBMC-derived, CD3+ purified T cells (primary T cells) or Jurkat E6.1 T cells were transduced with transposon vectors carrying our CAR in Example 1 constructs using electroporation.

### Example 3. Characterization of CAR T Cells Expressing Endocytic CAR (MLB002 and MLB003)

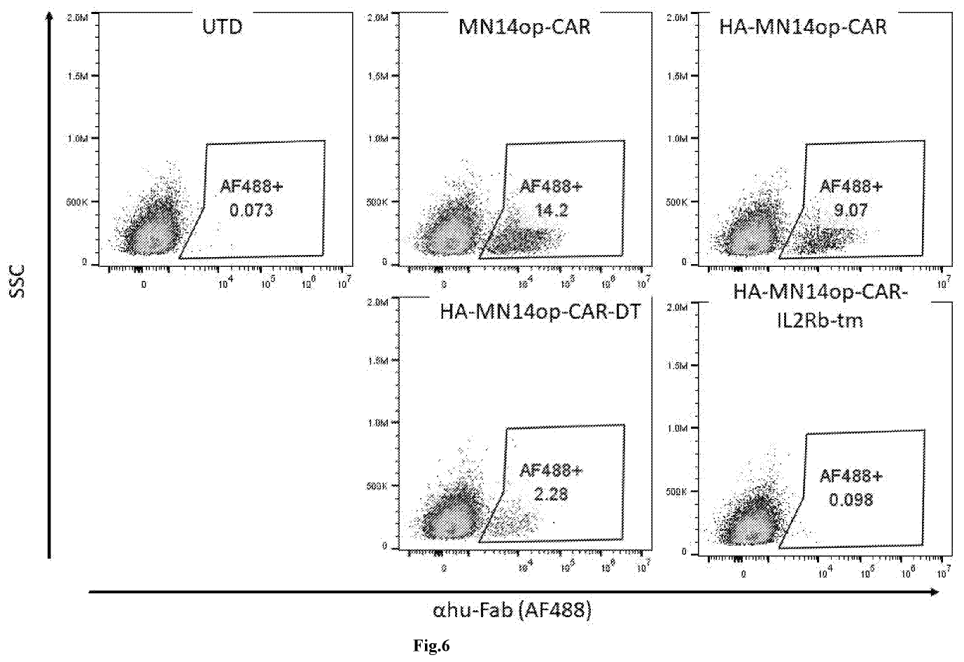

Transduced CAR-T cells were analyzed by flow cytometry to measure construct integration into the host cell. Cells were stained using anti-human Fab′ conjugated to AlexaFluor 488 (AF488). The endocytic CARs were also engineered with an extracellular, N-terminal HA tag to aid in detection of the receptor and perform pulse-chase experiments. CAR surface expression was detectable following electroporation of the constructs into Jurkat E6.1 cells (FIG. 4). In FIG. 4, the SSC of ordinate is a relative measure of cellular complexity, and pMAX-GFP is a control plasmid used to determine electroporation efficiency, particularly when a fluorescent marker is not available in the test construct. A linear correlation between anti-human Fab′ AF488 and HA staining was detected (FIG. 5). Expression of our endocytic CAR constructs in donor PBMC-derived T cells using anti-human Fab′ AF488 staining was also confirmed. Trends in receptor expression were comparable between primary T cells and Jurkat E6.1 cells (FIG. 6). The conclusion illustrated in FIGS. 4-6 is that HA-MN14op-CAR-IL2Rb-tm (MLB003) is similar to non-transduced (UTD), with lower staining value of anti-human Fab′ AF488 than the control CAR (HA-MN14op-CAR, MLB001), which reflects the least expression on the cell surface, indicating that good endocytosis effect is obtained by using IL2Rβ chain transmembrane, juxtamembrane motifs and DT sequence (PSKFFSQL). Compared with the constructs without DT sequence (HA-MN14op-CAR, MLB001), HA-MN14op-CAR-DT (MLB002) has lower expression, and also exhibits endocytosis, which was not observed with the constructs without DT sequence.

### Example 4. NFAT-Luciferase Reporter Assay for Endocytic CARs (MLB002 and MLB003)

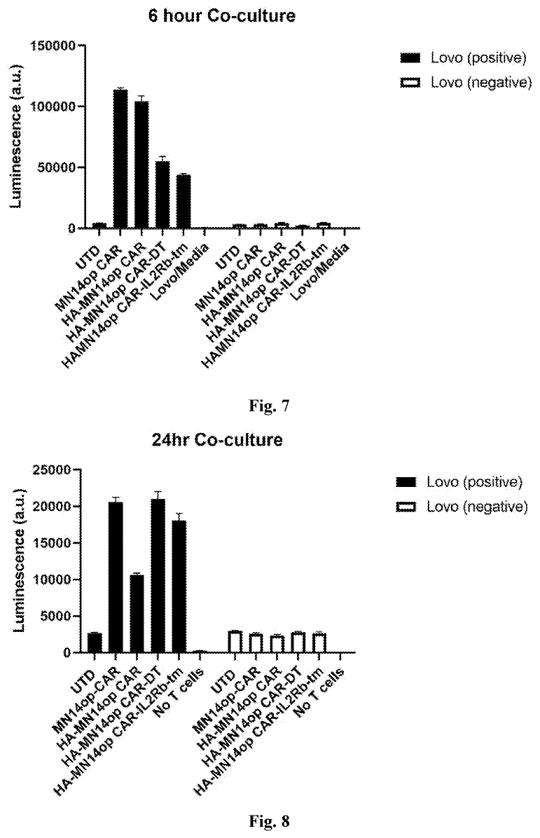

Jurkat E6.1 NFAT-Luciferase reporter cells expressing CAR Constructs were mixed 1:1 with antigen-expressing target cells (LoVo) and luciferase activity was measured after 6-hours (FIG. 7) or 24-hours of co-culture (FIG. 8). LoVo negative means that there are no LoVo cells mixed with Jurkat E6.1 cells. NFAT is a T cell transcription factor associated with T cell activation. Increased NFAT activity is associated with increased signal strength. Therefore, if we see increased luciferase signal, we know there is increased NFAT activity and increased CAR signal strength. FIGS. 7 and 8 show that in the cells expressing target antigen, the LoVo target antigen is CEA, and the endocytic CAR (MLB002 or MLB003) is activated, which has NFAT-driven luciferase activity. The luminescence value of the endocytic CAR is higher than non-transduced (UTD). Although the luminescence values of MLB002 and MLB003 after 6-hours co-culture were lower than those of the control, the values of MLB002 and MLB003 after 24-hours co-culture were higher than those of the control. Although surface expression of the endocytic CAR constructs was undetectable by flow cytometry (FIGS. 4 & 5), cells transduced with the endocytic CAR constructs showed NFAT-driven changes in gene expression and luciferase activity, confirming CAR functionality.

### Example 5. Cytotoxicity Assay for Endocytic CARs (MLB002 and MLB003)

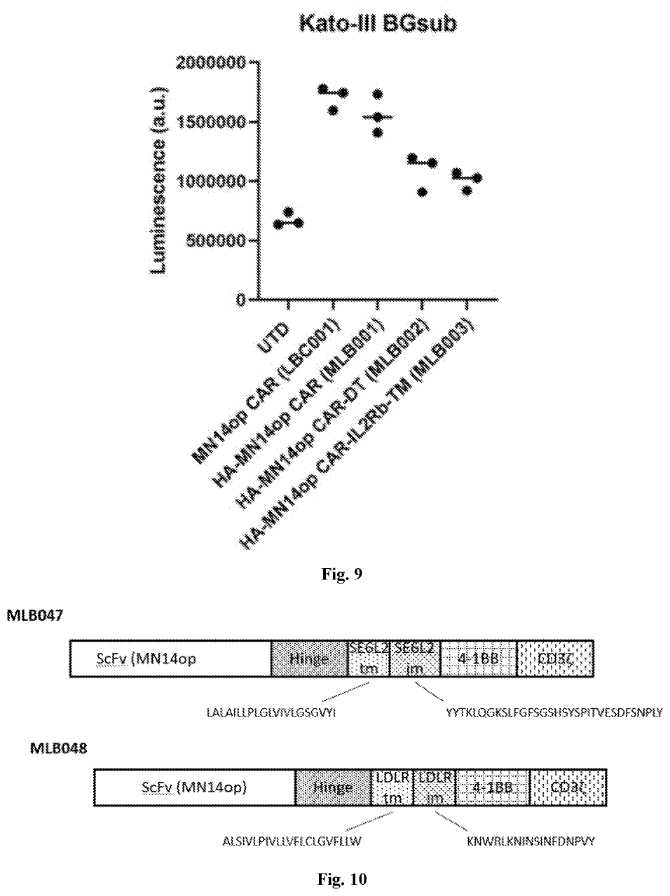

Donor-derived primary CD3+ cells (primary cells) transduced to express our CAR constructs were mixed 1:1 with antigen-expressing luciferase+ target cells (Kato-III, which express target antigen CEA) and co-cultured for 24 hours. Cytotoxicity was measured by increased luciferase activity (FIG. 9). Although endocytic CAR (MLB002 and MLB003) expression in primary cells was undetectable by flow cytometry (FIG. 6), cells transduced with the endocytic CAR showed enhanced cell-mediated killing relative to the untransduced control cells (UTD).

### Example 6. Non-Lentiviral Transfection of Jurkat and Primary CD3+ PBMCs with Endocytic CAR Constructs (MLB020, MLB013, MLB025, MLB048, MLB047, MLB054, and MLB055)

In this Example 6, CAR constructs were designed as follows:
(1) CAR construct containing a IL2Rβ transmembrane domain, a IL2Rβ juxtamembrane domain, and further containing a IL2Rβ degradation sequence (DT):
(a) MN14op CEA scFv-CD8Hinge-IL2Rβ tm jm DT-4-1BB-CD3ζ-P2A-GFP(MLB020);
(b) HA-MN14op CEA scFv-CD8Hinge-IL2Rβ tm jm DT-4-1BB-CD3ζ-P2A-GFP(MLB013).
(2) CAR construct containing cytoplasmic segment comprising an IL2Rβ degradation sequence (DT):
(a) MN14op CEA scFv-CD8Hinge-CD8 tm-4-1BB-CD3ζ-IL2R DT-P2A-GFP(MLB025).
(3) CAR construct containing an LDLR transmembrane domain and an LDLR juxtamembrane domain:
(a) MN14op CEA scFv-CD8Hinge-LDLR tm jm-4-1BB-CD3ζ-P2A-GFP(MLB048).
(4) CAR construct containing a SEZ6L2 transmembrane domain and a SEZ6L2 juxtamembrane domain:
(a) MN14op CEA scFv-CD8Hinge-SEZ6L2 tm jm-4-1BB-CD3ζ-P2A-GFP(MLB047);
(b) 841 Claudin 18.2 scFv-CD8Hinge-SEZ6L2 tm jm-4-1BB-CD3ζ-P2A-GFP(MLB054).
(5) The dual CAR construct:
(a) 841 Claudin 18.2 scFv-CD8Hinge-SEZ6L2 tm jm-4-1BB-CD3ζ-P2A-PD-L1 scFv-CD8Hinge-CD8 tm-CD28-(G₄S)₂-GFP(MLB055).
(6) The control CAR construct using CD8 transmembrane domain:
(a) MN14op CEA scFv-CD8Hinge-CD8 tm-4-1BB-CD3ζ-P2A-GFP (MLB010);
(b) 841 Claudin 18.2 scFv-CD8Hinge-CD8 tm-4-1BB-CD3ζ-P2A-GFP (MLB026).

The pMAX-GFP is a control plasmid used to determine electroporation efficiency, particularly when a fluorescent marker is not available in the test construct.

TABLE 2
The sequences used in Examples 6-10

| DESCRIPTION | | SEQ |
|---|---|---|
| (CAR construct) | SEQUENCE | ID NO: |
| MN14op CEA | MALPVTALLLPLALLLHAARPEVQLVESGGGVVKPGGSLRLSCS | 12 |
| scFv -CD8Hinge- | ASGFDFTTYWMSWVRQAPGKGLEWIGEIHPDSSTINYAPSLKDR | |
| CD8 tm-4-1BB- | FTISRDNAKNTLYLQMDSLRPEDTGVYYCASLYFGFPWFAYWG | |
| CD3ζ-P2A-GFP | QGTTVTVSSGGGGSGGGGSGGGGSGGGGSDIVLTQSPSSLSASLG | |
| (MLB010) | DRVTITCKASQDVGTSVAWYQQKPGKAPKLLIYWTSTRHTGVPS | |
| | RFSGSGSGTDFTFTISSLQPEDIATYYCQQYSLYRSFGGGTKVEIK | |
| | GTTTPAPRPPTPAPTIASQPLSLRPEACRPAAGGAVHTRGLDFAC | |
| | DIYIWAPLAGTCGVLLLSLVITLYCKRGRKKLLYIFKQPFMRPVQ | |
| | TTQEEDGCSCRFPEEEEGGCELRVKFSRSADAPAYKQGQNQLYN | |
| | ELNLGRREEYDVLDKRRGRDPEMGGKPRRKNPQEGLYNELQKD | |
| | KMAEAYSEIGMKGERRRGKGHDGLYQGLSTATKDTYDALHMQ | |
| | ALPPRGSGATNFSLLKQAGDVEENPGPMVSKGEELFTGVVPILVE | |
| | LDGDVNGHKFSVSGEGEGDATYGKLTLKFICTTGKLPVPWPTLV | |
| | TTLTYGVQCFSRYPDHMKQHDFFKSAMPEGYVQERTIFFKDDGN | |
| | YKTRAEVKFEGDTLVNRIELKGIDFKEDGNILGHKLEYNYNSHN | |
| | VYIMADKQKNGIKVNFKIRHNIEDGSVQLADHYQQNTPIGDGPV | |
| | LLPDNHYLSTQSALSKDPNEKRDHMVLLEFVTAAGITLGMDELY | |
| | K* | |
| 841 Claudin 18.2 | MALPVTALLLPLALLLHAARPQVQLVQSGAEVKKPGSSVKVSCK | 13 |
| scFv-CD8Hinge- | ASGGTFSSYAISWVRQAPGQGLEWMGRIIPILGIANYAQKFQGRV | |
| CD8 tm-4-1BB- | TVTADKSTSTAYMELSSLRSEDTAVYYCARERDNWDPYYYYYG | |
| CD3ζ-P2A-GFP | MDVWGQGTTVTVSSGGGGSGGGGSGGGGSDIQMTQSPSTLSAS | |
| (MLB026) | VGDRVTITCRASQSINSWLAWYQQKPGKAPKLLIYKASSLESGVP | |
| | SRFSGSGSGTEFTLTISSLQPDDFATYYCQQYNSFPLTFGGGTKVE | |
| | IKRTTTTPAPRPPTPAPTIASQPLSLRPEACRPAAGGAVHTRGLDF | |
| | ACDIYIWAPLAGTCGVLLLSLVITLYCKRGRKKLLYIFKQPFMRP | |
| | VQTTQEEDGCSCRFPEEEEGGCELRVKFSRSADAPAYKQGQNQL | |
| | YNELNLGRREEYDVLDKRRGRDPEMGGKPRRKNPQEGLYNELQ | |
| | KDKMAEAYSEIGMKGERRRGKGHDGLYQGLSTATKDTYDALH | |
| | MQALPPRGSGATNFSLLKQAGDVEENPGPMVSKGEELFTGVVPI | |
| | LVELDGDVNGHKFSVSGEGEGDATYGKLTLKFICTTGKLPVPWP | |
| | TLVTTLTYGVQCFSRYPDHMKQHDFFKSAMPEGYVQERTIFFKD | |
| | DGNYKTRAEVKFEGDTLVNRIELKGIDFKEDGNILGHKLEYNYN | |
| | SHNVYIMADKQKNGIKVNFKIRHNIEDGSVQLADHYQQNTPIGD | |
| | GPVLLPDNHYLSTQSALSKDPNEKRDHMVLLEFVTAAGITLGMD | |
| | ELYK* | |
| MN14op CEA | MALPVTALLLPLALLLHAARPEVQLVESGGGVVKPGGSLRLSCS | 14 |
| scFv-CD8Hinge- | ASGFDFTTYWMSWVRQAPGKGLEWIGEIHPDSSTINYAPSLKDR | |
| IL2Rβ tm jm DT- | FTISRDNAKNTLYLQMDSLRPEDTGVYYCASLYFGFPWFAYWG | |
| 4-1BB-CD3ζ-P2A- | QGTTVTVSSGGGGSGGGGSGGGGSGGGGSDIVLTQSPSSLSASLG | |
| GFP | DRVTITCKASQDVGTSVAWYQQKPGKAPKLLIYWTSTRHTGVPS | |
| (MLB020) | RFSGSGSGTDFTFTISSLQPEDIATYYCQQYSLYRSFGGGTKVEIK | |
| | GTTTPAPRPPTPAPTIASQPLSLRPEACRPAAGGAVHTRGLDFAC | |
| | DIPWLGHLLVGLSGAFGFIILVYLLINCRNTGPWLKKVLKCNTPD | |
| | PSKFFSQLKRGRKKLLYIFKQPFMRPVQTTQEEDGCSCRFPEEEE | |
| | GGCELRVKFSRSADAPAYKQGQNQLYNELNLGRREEYDVLDKR | |
| | RGRDPEMGGKPRRKNPQEGLYNELQKDKMAEAYSEIGMKGERR | |
| | RGKGHDGLYQGLSTATKDTYDALHMQALPPRGSGATNFSLLKQ | |
| | AGDVEENPGPMVSKGEELFTGVVPILVELDGDVNGHKFSVSGEG | |
| | EGDATYGKLTLKFICTTGKLPVPWPTLVTTLTYGVQCFSRYPDH | |
| | MKQHDFFKSAMPEGYVQERTIFFKDDGNYKTRAEVKFEGDTLV | |
| | NRIELKGIDFKEDGNILGHKLEYNYNSHNVYIMADKQKNGIKVN | |
| | FKIRHNIEDGSVQLADHYQQNTPIGDGPVLLPDNHYLSTQSALSK | |
| | DPNEKRDHMVLLEFVTAAGITLGMDELYK* | |
| HA-MN14op CEA | MALPVTALLLPLALLLHAARPYPYDVPDYAMEVOLVESGGGVV | 15 |
| scFv-CD8Hinge- | KPGGSLRLSCSASGFDFTTYWMSWVRQAPGKGLEWIGEIHPDSS | |
| IL2Rβ tm jm DT- | TINYAPSLKDRFTISRDNAKNTLYLQMDSLRPEDTGVYYCASLYF | |
| 4-1BB-CD3ζ-P2A- | GFPWFAYWGQGTTVTVSSGGGGSGGGGSGGGGSGGGGSDIVLT | |
| GFP | QSPSSLSASLGDRVTITCKASQDVGTSVAWYQQKPGKAPKLLIY | |
| (MLB013) | WTSTRHTGVPSRFSGSGSGTDFTFTISSLQPEDIATYYCQQYSLYR | |
| | SFGGGTKVEIKGTTTPAPRPPTPAPTIASQPLSLRPEACRPAAGGA | |
| | VHTRGLDFACDIPWLGHLLVGLSGAFGFIILVYLLINCRNTGPWL | |
| | KKVLKCNTPDPSKFFSQLKRGRKKLLYIFKQPFMRPVQTTQEED | |
| | GCSCRFPEEEEGGCELRVKFSRSADAPAYKQGQNQLYNELNLGR | |
| | REEYDVLDKRRGRDPEMGGKPRRKNPQEGLYNELQKDKMAEA | |
| | YSEIGMKGERRRGKGHDGLYQGLSTATKDTYDALHMQALPPRA | |
| | TNFSLLKQAGDVEENPGPMVSKGEELFTGVVPILVELDGDVNGH | |
| | KFSVSGEGEGDATYGKLTLKFICTTGKLPVPWPTLVTTLTYGVQ | |
| | CFSRYPDHMKQHDFFKSAMPEGYVQERTIFFKDDGNYKTRAEV | |
| | KFEGDTLVNRIELKGIDFKEDGNILGHKLEYNYNSHNVYIMADK | |
| | QKNGIKVNFKIRHNIEDGSVQLADHYQQNTPIGDGPVLLPDNHY | |
| | LSTQSALSKDPNEKRDHMVLLEFVTAAGITLGMDELYK* | |
| MN14op CEA | MALPVTALLLPLALLLHAARPEVQLVESGGGVVKPGGSLRLSCS | 16 |
| scFv-CD8Hinge- | ASGFDFTTYWMSWVRQAPGKGLEWIGEIHPDSSTINYAPSLKDR | |
| CD8 tm-4-1BB- | FTISRDNAKNTLYLQMDSLRPEDTGVYYCASLYFGFPWFAYWG | |
| CD3ζ-IL2Rß DT - | QGTTVTVSSGGGGSGGGGSGGGGSGGGGSDIVLTQSPSSLSASLG | |
| P2A-GFP | DRVTITCKASQDVGTSVAWYQQKPGKAPKLLIYWTSTRHTGVPS | |
| (MLB025) | RFSGSGSGTDFTFTISSLQPEDIATYYCQQYSLYRSFGGGTKVEIK | |
| | GTTTPAPRPPTPAPTIASQPLSLRPEACRPAAGGAVHTRGLDFAC | |
| | DIYIWAPLAGTCGVLLLSLVITLYCKRGRKKLLYIFKQPFMRPVQ | |
| | TTQEEDGCSCRFPEEEEGGCELRVKFSRSADAPAYKQGQNQLYN | |
| | ELNLGRREEYDVLDKRRGRDPEMGGKPRRKNPQEGLYNELQKD | |
| | KMAEAYSEIGMKGERRRGKGHDGLYQGLSTATKDTYDALHMQ | |
| | ALPPRPSKFFSQLGSGATNFSLLKQAGDVEENPGPMVSKGEELF | |
| | TGVVPILVELDGDVNGHKFSVSGEGEGDATYGKLTLKFICTTGK | |
| | LPVPWPTLVTTLTYGVQCFSRYPDHMKQHDFFKSAMPEGYVQE | |
| | RTIFFKDDGNYKTRAEVKFEGDTLVNRIELKGIDFKEDGNILGHK | |
| | LEYNYNSHNVYIMADKQKNGIKVNFKIRHNIEDGSVQLADHYQ | |
| | QNTPIGDGPVLLPDNHYLSTQSALSKDPNEKRDHMVLLEFVTAA | |
| | GITLGMDELYK* | |
| MN14op CEA | MALPVTALLLPLALLLHAARPEVQLVESGGGVVKPGGSLRLSCS | 17 |
| scFv-CD8Hinge- | ASGFDFTTYWMSWVRQAPGKGLEWIGEIHPDSSTINYAPSLKDR | |
| SEZ6L2 tm jm-4- | FTISRDNAKNTLYLQMDSLRPEDTGVYYCASLYFGFPWFAYWG | |
| 1BB-CD3ζ-P2A- | QGTTVTVSSGGGGSGGGGSGGGGSGGGGSDIVLTQSPSSLSASLG | |
| GFP | DRVTITCKASQDVGTSVAWYQQKPGKAPKLLIYWTSTRHTGVPS | |
| (MLB047) | RFSGSGSGTDFTFTISSLQPEDIATYYCQQYSLYRSFGGGTKVEIK | |
| | GTTTPAPRPPTPAPTIASQPLSLRPEACRPAAGGAVHTRGLDFAC | |
| | DLALAILLPLGLVIVLGSGVYIYYTKLQGKSLFGFSGSHSYSPITV | |
| | ESDFSNPLYKRGRKKLLYIFKQPFMRPVQTTQEEDGCSCRFPEEE | |
| | EGGCELRVKFSRSADAPAYKQGQNQLYNELNLGRREEYDVLDK | |
| | RRGRDPEMGGKPRRKNPQEGLYNELQKDKMAEAYSEIGMKGER | |
| | RRGKGHDGLYQGLSTATKDTYDALHMQALPPRGSGATNFSLLK | |
| | QAGDVEENPGPMVSKGEELFTGVVPILVELDGDVNGHKFSVSGE | |
| | GEGDATYGKLTLKFICTTGKLPVPWPTLVTTLTYGVQCFSRYPD | |
| | HMKQHDFFKSAMPEGYVQERTIFFKDDGNYKTRAEVKFEGDTL | |
| | VNRIELKGIDFKEDGNILGHKLEYNYNSHNVYIMADKQKNGIKV | |
| | NFKIRHNIEDGSVQLADHYQQNTPIGDGPVLLPDNHYLSTQSALS | |
| | KDPNEKRDHMVLLEFVTAAGITLGMDELYK* | |
| MN14op CEA | MALPVTALLLPLALLLHAARPEVQLVESGGGVVKPGGSLRLSCS | 18 |
| scFv-CD8Hinge- | ASGFDFTTYWMSWVRQAPGKGLEWIGEIHPDSSTINYAPSLKDR | |
| LDLR tm jm-4- | FTISRDNAKNTLYLQMDSLRPEDTGVYYCASLYFGFPWFAYWG | |
| 1BB-CD3ζ-P2A- | QGTTVTVSSGGGGSGGGGSGGGGSGGGGSDIVLTQSPSSLSASLG | |
| GFP | DRVTITCKASQDVGTSVAWYQQKPGKAPKLLIYWTSTRHTGVPS | |
| (MLB048) | RFSGSGSGTDFTFTISSLQPEDIATYYCQQYSLYRSFGGGTKVEIK | |
| | GTTTPAPRPPTPAPTIASQPLSLRPEACRPAAGGAVHTRGLDFAC | |
| | DALSIVLPIVLLVFLCLGVFLLWKNWRLKNINSINFDNPVYKRGR | |
| | KKLLYIFKQPFMRPVQTTQEEDGCSCRFPEEEEGGCELRVKFSRS | |
| | ADAPAYKQGQNQLYNELNLGRREEYDVLDKRRGRDPEMGGKP | |
| | RRKNPQEGLYNELQKDKMAEAYSEIGMKGERRRGKGHDGLYQ | |
| | GLSTATKDTYDALHMQALPPRGSGATNFSLLKQAGDVEENPGP | |
| | MVSKGEELFTGVVPILVELDGDVNGHKFSVSGEGEGDATYGKLT | |
| | LKFICTTGKLPVPWPTLVTTLTYGVQCFSRYPDHMKQHDFFKSA | |
| | MPEGYVQERTIFFKDDGNYKTRAEVKFEGDTLVNRIELKGIDFKE | |
| | DGNILGHKLEYNYNSHNVYIMADKQKNGIKVNFKIRHNIEDGSV | |
| | QLADHYQQNTPIGDGPVLLPDNHYLSTQSALSKDPNEKRDHMVL | |
| | LEFVTAAGITLGMDELYK* | |
| 841 Claudin 18.2 | MALPVTALLLPLALLLHAARPQVQLVQSGAEVKKPGSSVKVSCK | 19 |
| scFv-CD8Hinge- | ASGGTFSSYAISWVRQAPGQGLEWMGRIIPILGIANYAQKFQGRV | |
| SEZ6L2 tm jm-4- | TVTADKSTSTAYMELSSLRSEDTAVYYCARERDNWDPYYYYYG | |
| 1BB-CD3ζ-P2A- | MDVWGQGTTVTVSSGGGGSGGGGSGGGGSDIQMTQSPSTLSAS | |
| GFP | VGDRVTITCRASQSINSWLAWYQQKPGKAPKLLIYKASSLESGVP | |
| (MLB054) | SRFSGSGSGTEFTLTISSLQPDDFATYYCQQYNSFPLTFGGGTKVE | |
| | IKRTTTTPAPRPPTPAPTIASQPLSLRPEACRPAAGGAVHTRGLDF | |
| | ACDLALAILLPLGLVIVLGSGVYIYYTKLQGKSLFGFSGSHSYSPI | |
| | TVESDFSNPLYKRGRKKLLYIFKQPFMRPVQTTQEEDGCSCRFPE | |
| | EEEGGCELRVKFSRSADAPAYKQGQNQLYNELNLGRREEYDVL | |
| | DKRRGRDPEMGGKPRRKNPQEGLYNELQKDKMAEAYSEIGMK | |
| | GERRRGKGHDGLYQGLSTATKDTYDALHMQALPPRGSGATNFS | |
| | LLKQAGDVEENPGPMVSKGEELFTGVVPILVELDGDVNGHKFSV | |
| | SGEGEGDATYGKLTLKFICTTGKLPVPWPTLVTTLTYGVQCFSRY | |
| | PDHMKQHDFFKSAMPEGYVQERTIFFKDDGNYKTRAEVKFEGD | |
| | TLVNRIELKGIDFKEDGNILGHKLEYNYNSHNVYIMADKQKNGI | |
| | KVNFKIRHNIEDGSVQLADHYQQNTPIGDGPVLLPDNHYLSTQSA | |
| | LSKDPNEKRDHMVLLEFVTAAGITLGMDELYK* | |
| 841 Claudin 18.2 | MALPVTALLLPLALLLHAARPQVQLVQSGAEVKKPGSSVKVSCK | 20 |
| scFv-CD8Hinge- | ASGGTFSSYAISWVRQAPGQGLEWMGRIIPILGIANYAQKFQGRV | |
| SEZ6L2 tm jm-4- | TVTADKSTSTAYMELSSLRSEDTAVYYCARERDNWDPYYYYYG | |
| 1BB-CD3ζ-P2A- | MDVWGQGTTVTVSSGGGGSGGGGSGGGGSDIQMTQSPSTLSAS | |
| PD-L1 scFv- | VGDRVTITCRASQSINSWLAWYQQKPGKAPKLLIYKASSLESGVP | |
| CD8Hinge-CD8 | SRFSGSGSGTEFTLTISSLQPDDFATYYCQQYNSFPLTFGGGTKVE | |
| tm-CD28-(G4S)2- | IKRTTTTPAPRPPTPAPTIASQPLSLRPEACRPAAGGAVHTRGLDF | |
| GFP | ACDLALAILLPLGLVIVLGSGVYIYYTKLQGKSLFGFSGSHSYSPI | |
| (MLB055) | TVESDFSNPLYKRGRKKLLYIFKQPFMRPVQTTQEEDGCSCRFPE | |
| | EEEGGCELRVKFSRSADAPAYKQGQNQLYNELNLGRREEYDVL | |
| | DKRRGRDPEMGGKPRRKNPQEGLYNELQKDKMAEAYSEIGMK | |
| | GERRRGKGHDGLYQGLSTATKDTYDALHMQALPPRGSGATNFS | |
| | LLKQAGDVEENPGPMALPVTALLLPLALLLHAARPEVQLVQSGG | |
| | GVVQPGRSLRLSCAASGFTFSNYAMHWVRQAPGKGLEWVAIIT | |
| | YAGSNEYYADSVKGRFTISRDNSKNTLYLQMNSLRPEDTAVYYC | |
| | ARDRIWVDYWGQGTLVTVSSGGGGSGGGGSGGGGSDIQMTQSP | |
| | DSLAVSLGERATINCKSSQSVLYSSNNKNYLAWYQQKPGQPPKL | |
| | LIYWASTRESGVPDRFSGSGSGTDFTLTISSLQAEDVAVYYCQQY | |
| | YSTPLTFGGGTKVEIKTTTPAPRPPTPAPTIASQPLSLRPEACRPAA | |
| | GGAVHTRGLDFACDIYIWAPLAGTCGVLLLSLVITLYCRSKRSRL | |
| | LHSDYMNMTPRRPGPTRKHYQPYAPPRDFAAYRSGGGGSGGGG | |
| | SMVSKGEELFTGVVPILVELDGDVNGHKFSVSGEGEGDATYGKL | |
| | TLKFICTTGKLPVPWPTLVTTLTYGVQCFSRYPDHMKQHDFFKS | |
| | AMPEGYVQERTIFFKDDGNYKTRAEVKFEGDTLVNRIELKGIDF | |
| | KEDGNILGHKLEYNYNSHNVYIMADKQKNGIKVNFKIRHNIEDG | |
| | SVQLADHYQQNTPIGDGPVLLPDNHYLSTQSALSKDPNEKRDHM | |
| | VLLEFVTAAGITLGMDELYK* | |

Internalization Motif is bold and underlined.

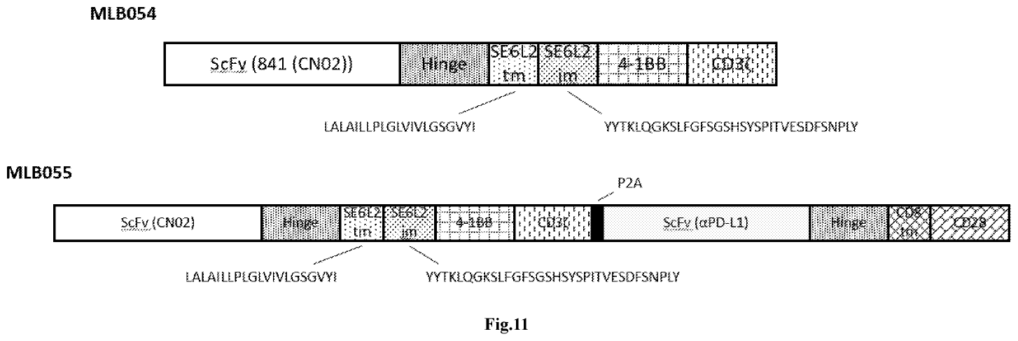

The T cell line, Jurkat E6.1, expressing an NFAT-driven luciferase reporter, and donor-derived T cells were induced to express our CAR constructs by electroporation of a Piggy Bac transposon plasmid with the CAR construct under control of the EF1α promoter and with transposase mRNA. FIGS. 10 and 11 show two cartoon schematic of the CAR constructs illustrating alterations to the transmembrane and juxtamembrane sequences. CARs MLB047 and MLB054 utilize the MN14op CEA ScFv and 841 Claudin 18.2 ScFv (CN02) antigen recognition domains, respectively. These CARs are designed with the Seizure-like 6 protein 2 (SEZ6L2) transmembrane and 33 amino acids from the juxtamembrane sequence. MLB048 utilizes the LDLR transmembrane and juxtamembrane sequences. MLB055 is a two-component CAR (dual CAR) based on the MLB054 Claudin18.2 CAR sequence and anti-PDL1 coupled to the CD28 signaling domain with the same CD8 hinge and transmembrane domains as the original CAR.

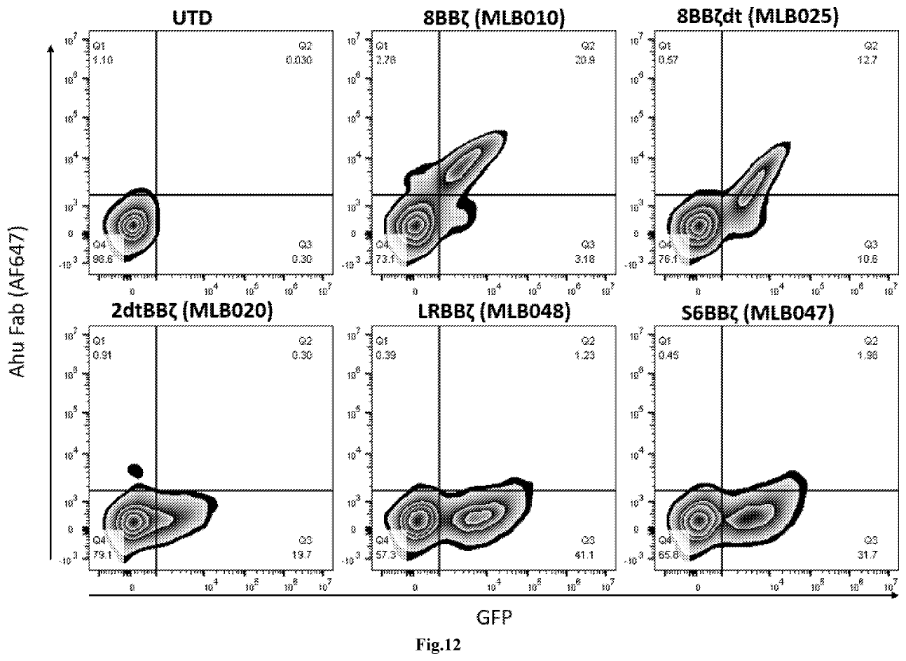

CAR surface expression was evaluated by flow cytometry through staining with AF647-conjugated αhuman Fab′ antibody; overall CAR construct expression was determined by co-expression of GFP under a P2A sequence (FIG. 12). Given the undetectable surface expression of CAR constructs designed using the IL2Rβ, LDLR, and Seizure 6-like Protein 2 transmembrane and juxtamembrane domains, GFP expression was used to normalize all subsequent assays on the fraction of CAR-positive cells. FIG. 12 shows that CAR constructs (MLB025, MLB 020, MLB048 and MLB047) using the IL2Rβ, LDLR, and SEZ6L2 transmembrane and juxtamembrane domains, when stained with AF674, exhibit staining values lower than control CAR construct MLB010, proving that the endocytic CAR is not expressed on the cell surface when there is no antigen target. The staining values of AF674 and GFP for control CAR construct MLB010 are higher.

### Example 7. Evaluation of CAR Construct Activity by Luciferase Reporter Activity and CD69 Upregulation

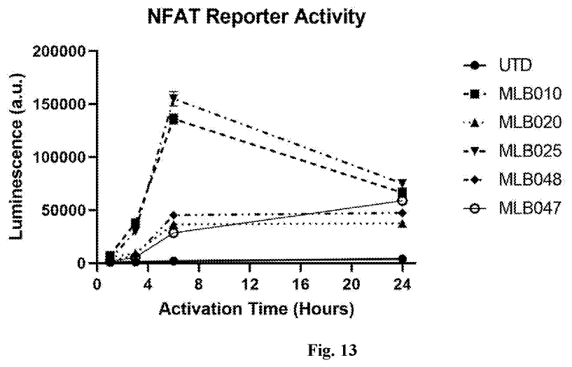

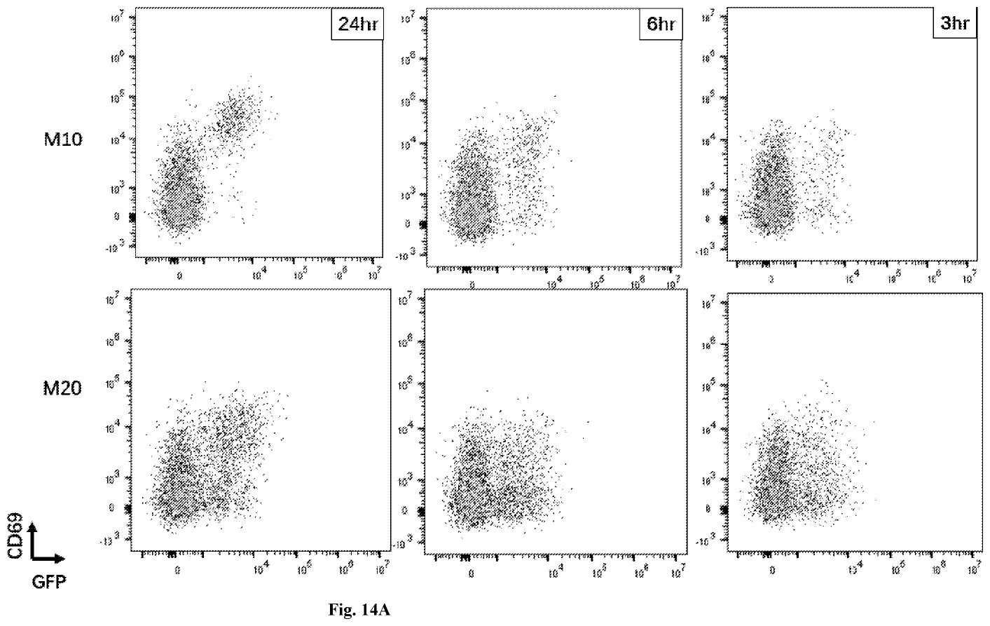

LoVo cells expressing the CEACAM-5 target antigen were cultured in white, opaque flat-bottom 96-well plates in the presence of 1 μg/mL of Mitomycin C to inhibit cell proliferation. 25,000 LoVo target cells were seeded per well 6-8 hours prior to the initiation of the time course assay to allow for adherence. The assay was performed at an E:T ratio of 1:1 with CAR+ Jurkat T cells. After 8 hours of incubation, the wells were washed, and media replenished. 24, 6, 3, and 1 hour prior to luminescence readout, the wells were washed and Jurkat T cells expressing the CAR constructs were added to each well. At the end of the 24-hour period, Bio-Glo™ reagent (Promega) was added to each well to assess overall Jurkat cell activation (FIG. 13).

Observation of the luminescent signal indicated that the activation kinetics of all of the CAR variants were similar and comparable to the original CAR construct, with NFAT-driven gene expression occurring strongly around 6 hours of activation. Although the kinetics of activation were comparable, the degree of cellular activation was strongest in the original (MLB010) and c-terminal (MLB025) tagged versions of the CARs. Both the LDLR- and SEZ6L2-based variants (MLB047 and MLB048) showed much lower levels of overall activation, and the IL2Rβ-based construct (MLB020) showed the lowest levels of expression (FIG. 13).

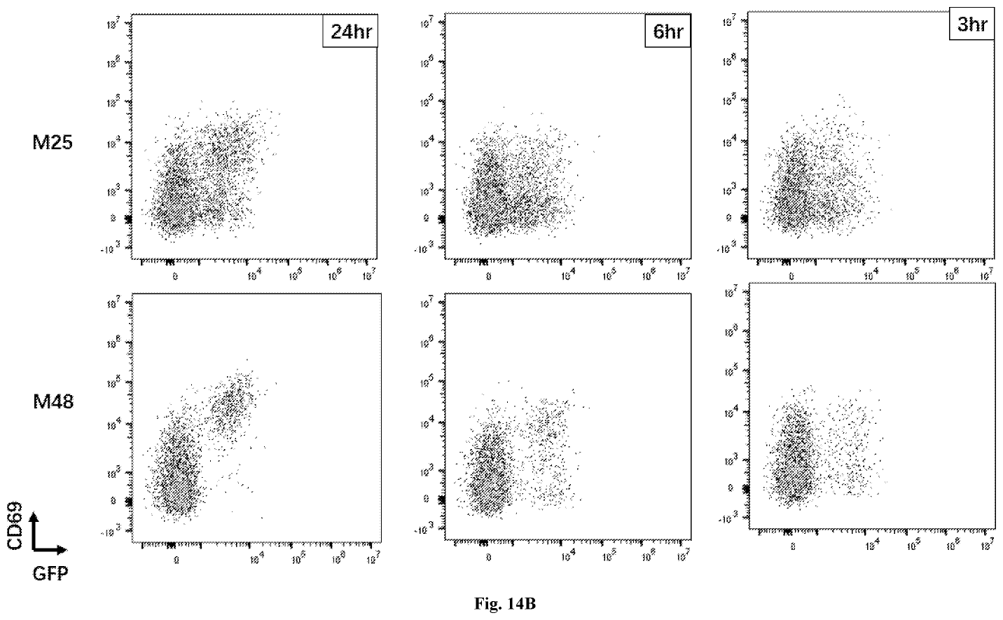

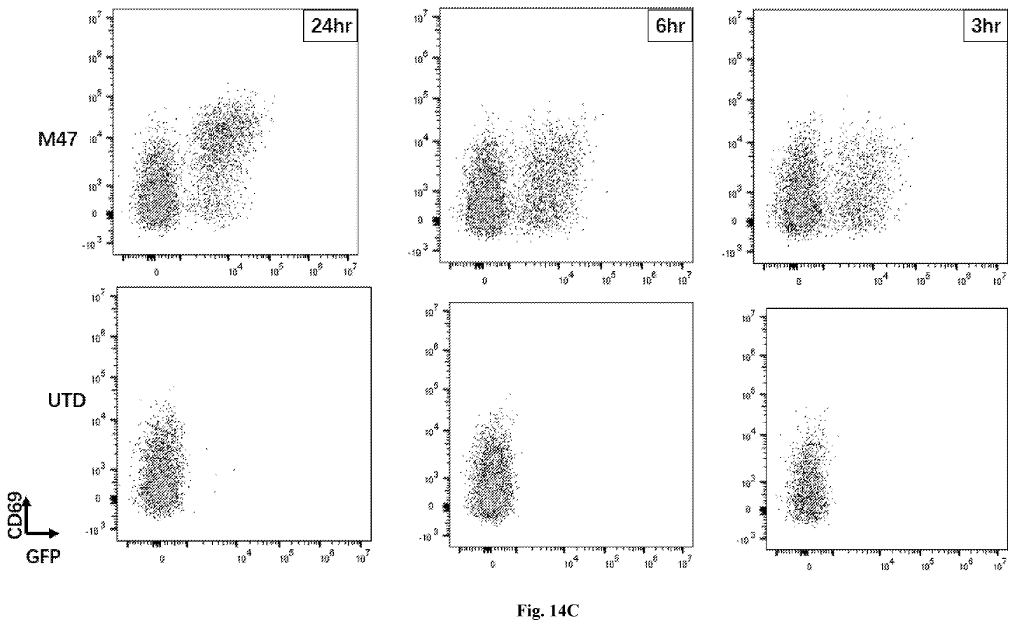

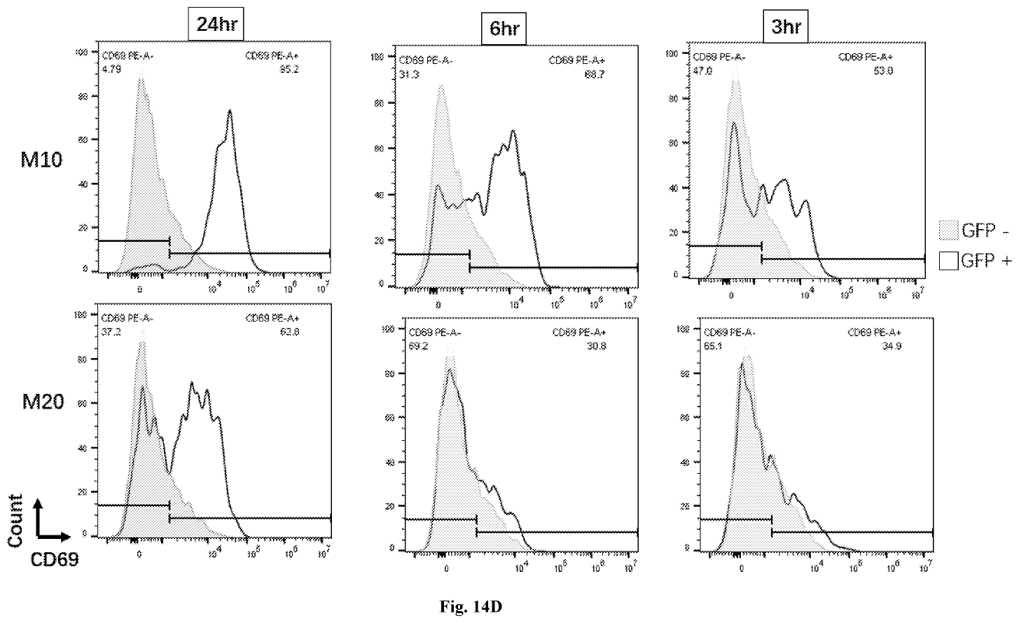

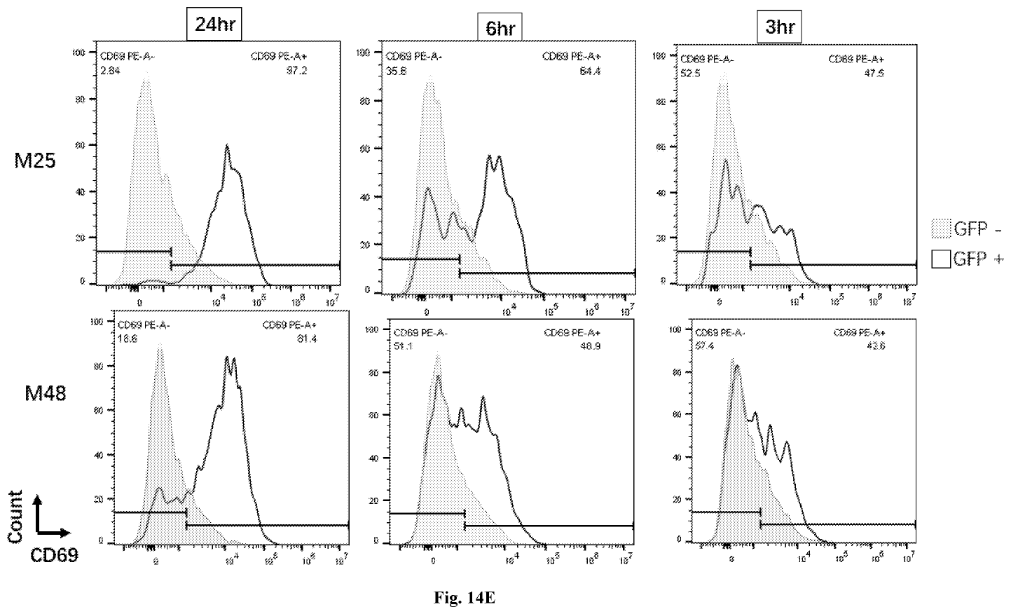

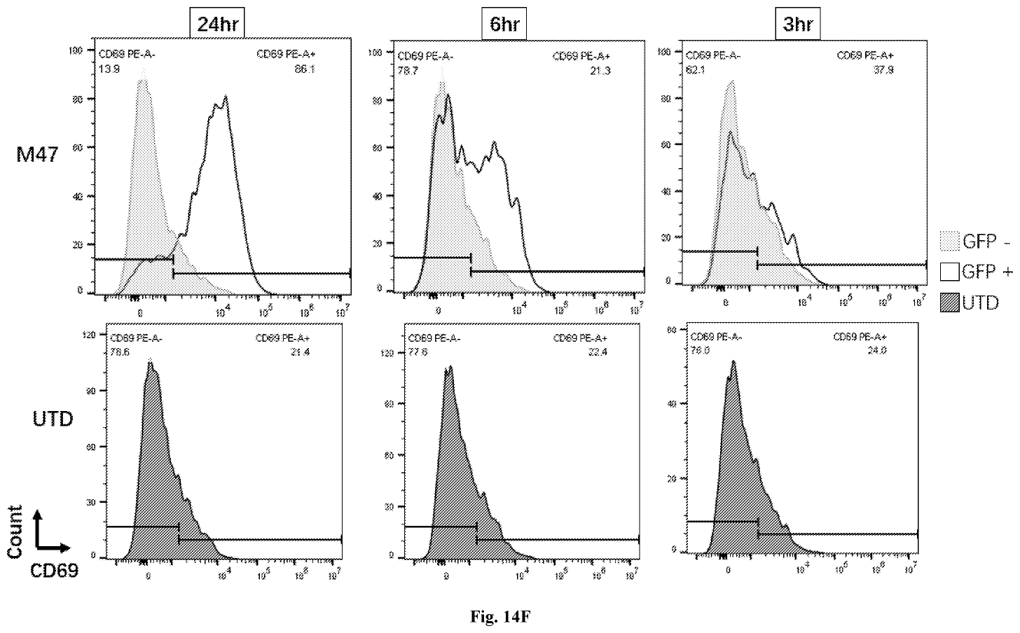

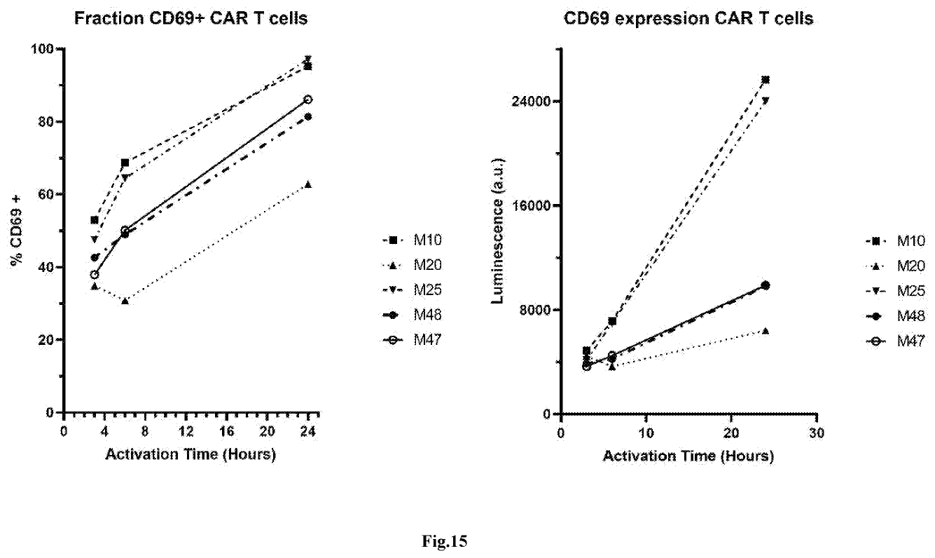

To further determine the effect of the various transmembrane domains on the overall activation state of the Jurkat T cells, we performed a second co-culture assay to determine the extent of CD69 upregulation following stimulation. 50,000 LoVo Target cells were co-cultured with Jurkat CAR-positive T cells at a 1:1 ratio for 3, 6, or 24 hours and CD69 expression was evaluated by flow cytometry. By gating the cells on GFP expression, we were able to determine the fraction of CD69-expressing cells in the CAR-positive population and the overall degree of CD69 upregulation by assessing fluorescence intensity (FIG. 14A-F). FIG. 14 A-F shows that both endocytic (MLB020, MLB025, MLB048 and MLB047) and non-endocytic CARs (MLB010) have CD69 upregulation. FIG. 14 A-C shows CAR positive population, and FIG. 14D-F shows the overall degree of CD69 upregulation. Using this information, we were able to generate curves illustrating the activation state of the CAR-positive cells to better compare between constructs (FIG. 15). In FIG. 14 A-F and FIG. 15, M10 represents MLB010, M20 represents MLB020, M25 represents MLB025, M48 represents MLB048, and M47 represents MLB047. Our data indicate that although the CD69 activation kinetics are analogous between all of the CAR constructs, those expressing the IL2Rβ, LDLR, or SEZ6L2 or domains (MLB020, MLB025, MLB048 and MLB047) showed a lower number of responding cells at any given time point as well as lower overall CD69 expression (FIG. 15). These results indicate that the novel receptor variants, despite showing no detectable CAR surface expression in the absence of antigen, are able to drive T cell activation. These findings also suggest a dampened activation response in MLB020, MLB025, MLB047, and MLB048 relative to MLB010 caused by the unique distribution of the receptor within the cell.

### Example 8. Cytotoxicity Assay for CAR MLB013, MLB020, MLB047, and MLB048

To assess the effect of the IL2Rβ (MLB013), SEZ6L2 (MLB047), and LDLR (MLB048) domains on downstream T cell activity, cytotoxicity assays against CEACAM-5-positive target cell lines LoVo, A549, and HT-29 were set up. These target cells lines were selected as they have high, intermediate, and low levels of CEA expression, respectively. Given that CAR target antigens often have variable expression between the tumor tissue and noncancerous tissue, this assay allows us to assess the potential for on-target, off-tumor CAR activity, which can cause severe side effects in a clinical setting.

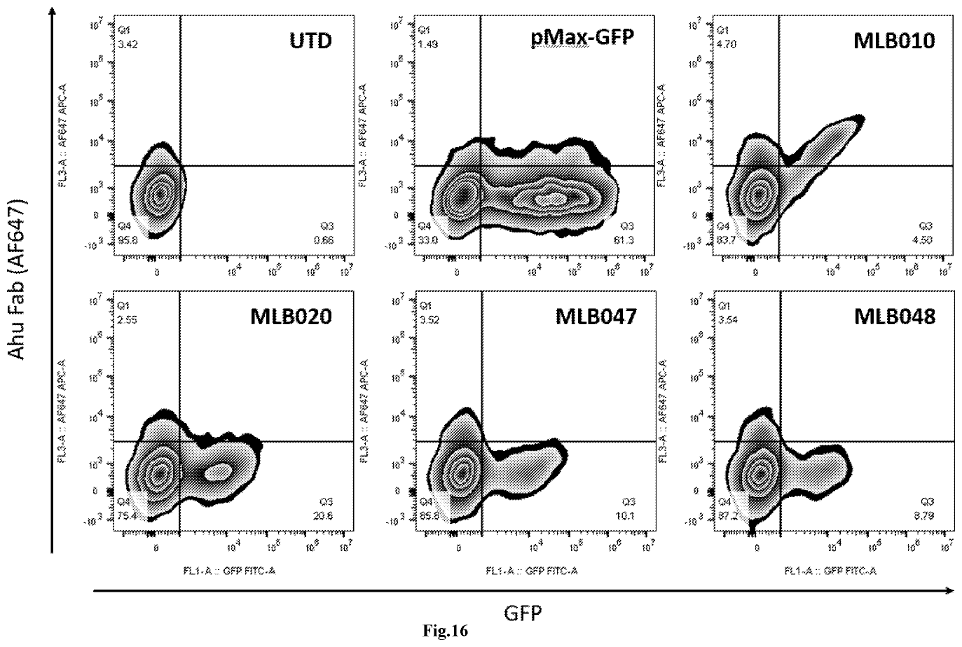

Donor-derived CD3+ PBMCs were transduced to express CAR constructs using the PiggyBac Expression system. Following electroporation, CAR expression was evaluated via flow cytometry by co-expression of GFP on the CAR expression vector and used to normalize cell numbers to directly compare CAR-T cell activity (FIG. 16). FIG. 16 shows the results of staining MLB020, MLB047, and MLB048 CAR constructs using the IL2Rβ, LDLR, and SEZ6L2 transmembrane and juxtamembrane domains with AF674, exhibiting staining values lower than control CAR construct MLB010, proving that the endocytic CAR is not expressed on the cell surface when there is no antigen target. The staining values of AF674 for the control CAR construct MLB010 are higher. Antigen-expressing, luciferase-positive target cells were seeded at a density of 10,000 cells per well and the assay was performed at three E:T ratios; 3:1, 1:1, 0.3:1, CAR-positive cells to target cells. Effector and target cells were co-cultured for 24 hours and cytotoxicity was determined as a decrease in luciferase activity, concurrent with target cell death.

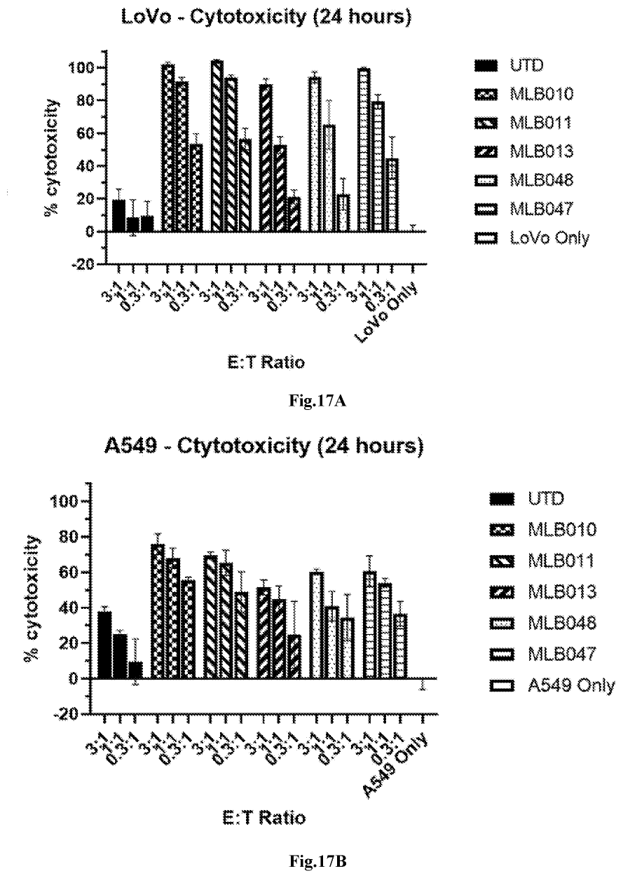

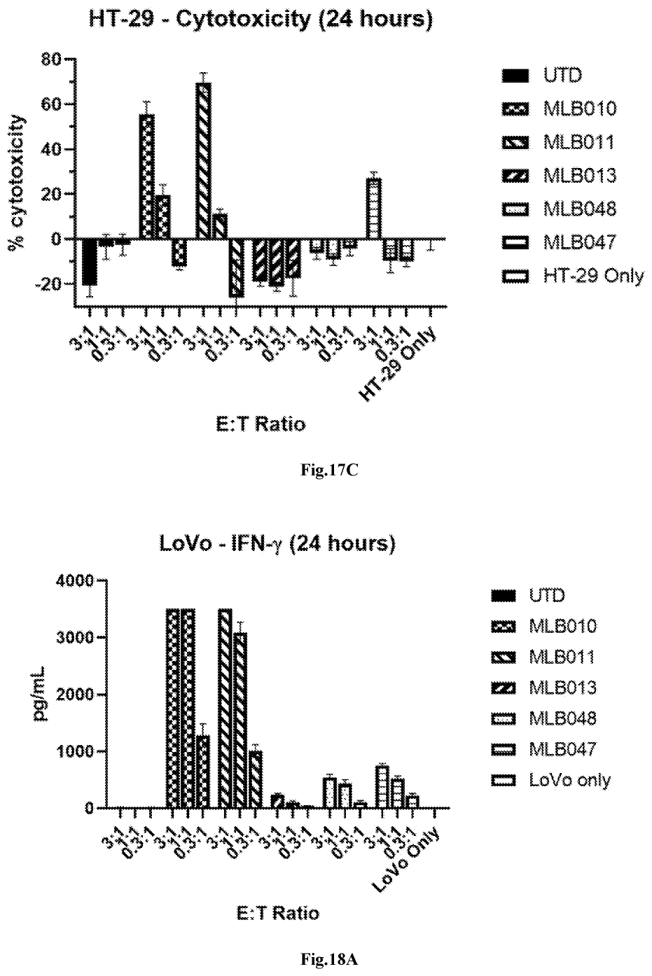

As expected, cytotoxic activity was directly related to the amount of antigen expression on the target cell lines, with the highest amount of cytotoxicity detected against LoVo Cells, and the lowest amount of cytotoxicity detected against HT-29 cells (FIG. 17A-C). Also as expected, cytotoxicity scaled with the number of effector cells per well, with a higher E:T ratio resulting in more cytotoxicity. Interestingly, at a 3:1 E:T ratio, MLB013, MLB047, and MLB048 performed similarly to the parental CAR (MLB010 and MLB011, MLB011 has an added HA sequence at MN14op CEA scFv N-terminal of MLB010) against cells highly expressing the target antigen, but as the E:T ratio was decreased, the cytotoxic potential decreased more rapidly in the MLB013, MLB047, and MLB048 variants. Of note, only MLB048 with SEZ6L2 retained cytotoxic activity against the antigen-low HT-29 cells. These results give the confidence that the addition of an endocytosis targeting motif to the CAR construct can expand the therapeutic window of the CAR construct.

### Example 9. Cytokine Expression Analysis for CAR MLB013, MLB047, and MLB048

Having shown that the novel transmembrane CAR constructs were able to retain cytotoxic activity against antigen-expressing target cells, we wanted to measure additional readouts of CAR T cell activity. Two common markers of activation are IL-2 and IFN-7. To assay for cytokine secretion, media was harvested following overnight cytotoxicity assays and soluble cytokine expression was determined via ELISA.

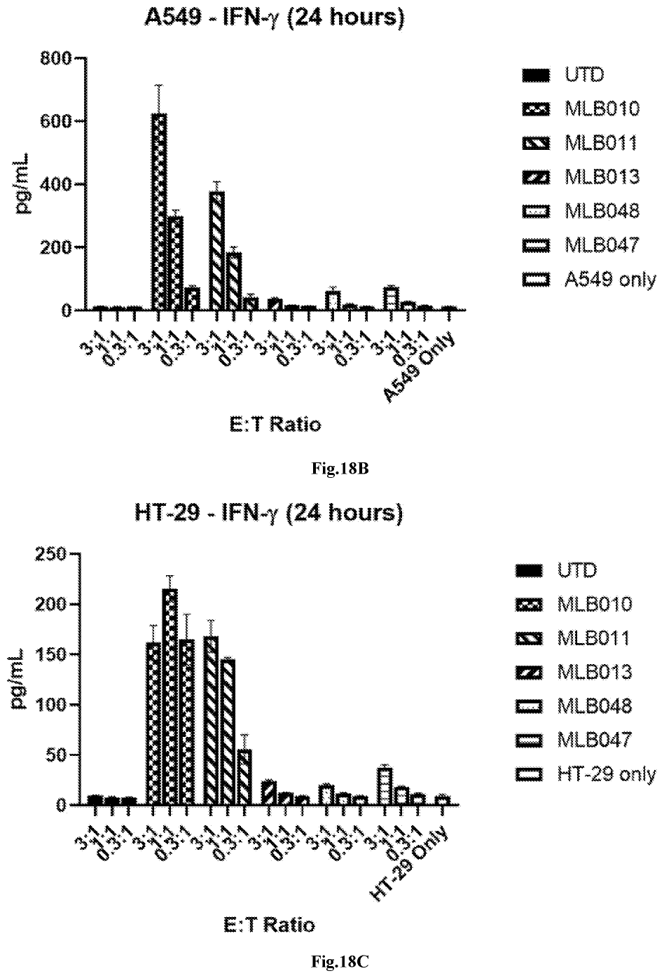

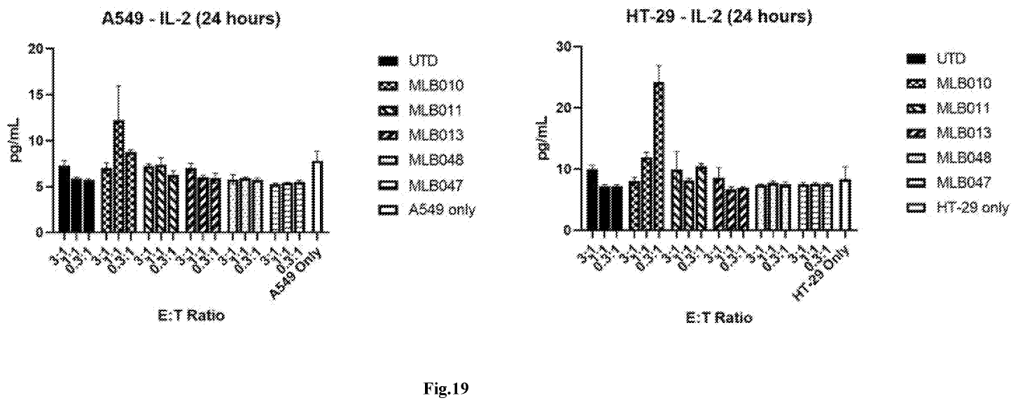

The trends that were observed in cytokine secretion mirrored those that we observed in cytotoxicity, with the highest levels of cytokine secretion detected against CEA-high target cells, and greatly reduced cytokine expression detected against HT-29 cells (FIG. 18A-C & FIG. 19). Interestingly cytokine secretion was almost undetectable for MLB013, MLB047, and MLB048, with MLB047 showing marginally higher cytokine expression relative to MLB013 and MLB048.

These findings are significant as CAR T cell therapies are known to promote potentially fatal Cytokine Release Syndrome (CRS). For this reason, the results are of clinical interest and indicate that the cytokine profile of CAR T cells can be tuned through the use of alternative transmembrane and juxtamembrane domains. By using the transmembrane domains and associated trafficking motifs of receptors that are known to be targeted towards the endosomal pathway, the results have shown that CAR T cell activity can be tuned, expanding the therapeutic window.

### Example 10. Claudin18.2-Specific and Augmented CAR T Cell Activation in Jurkat E6.1 Cells

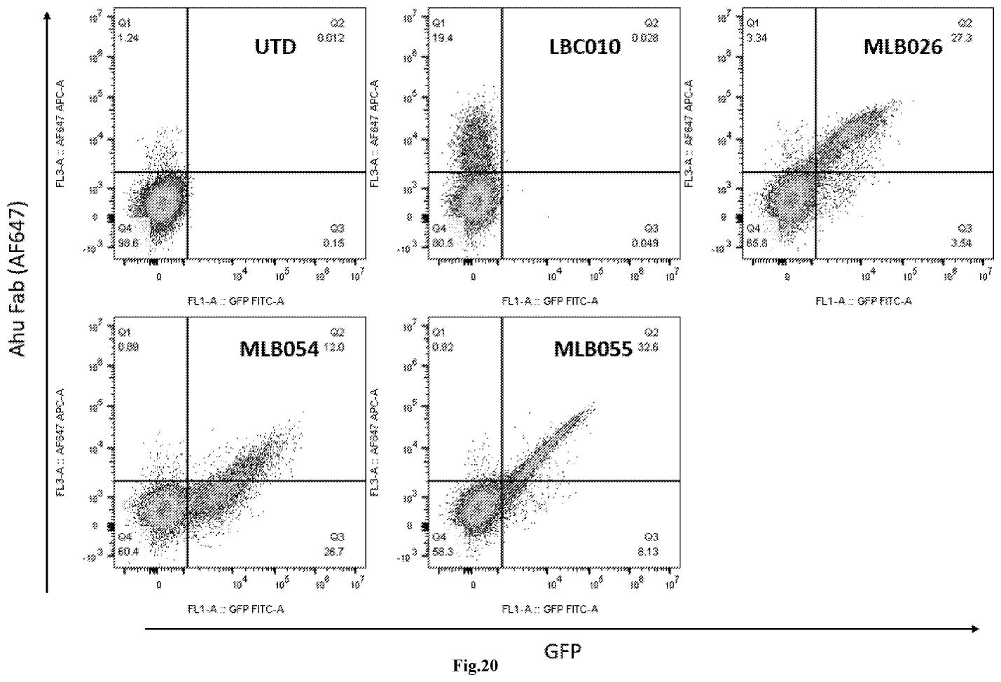

Jurkat E6.1 T cells expressing an NFAT-driven luciferase reporter were electroporated with the Piggy Bac vector expressing Claudin18.2-specific CAR constructs: MLB026, MLB054, MLB055; and Piggy Bac transposase mRNA. As before, CAR expression was evaluated by surface staining with AF647-conjugated αhuman-Fab′ antibody and co-expression of GFP on the CAR vector via a P2A sequence (FIG. 20). As before, inclusion of the SEZ6L2 transmembrane domain in the CAR sequence (MLB054) resulted in decreased CAR surface expression, while overall CAR expression as determined by GFP remained high. A strong correlation between GFP expression and αhuman-Fab′ staining in MLB055 due to the presence of the scFv PD-L1-CD28-GFP co-receptor in the construct was also observed.

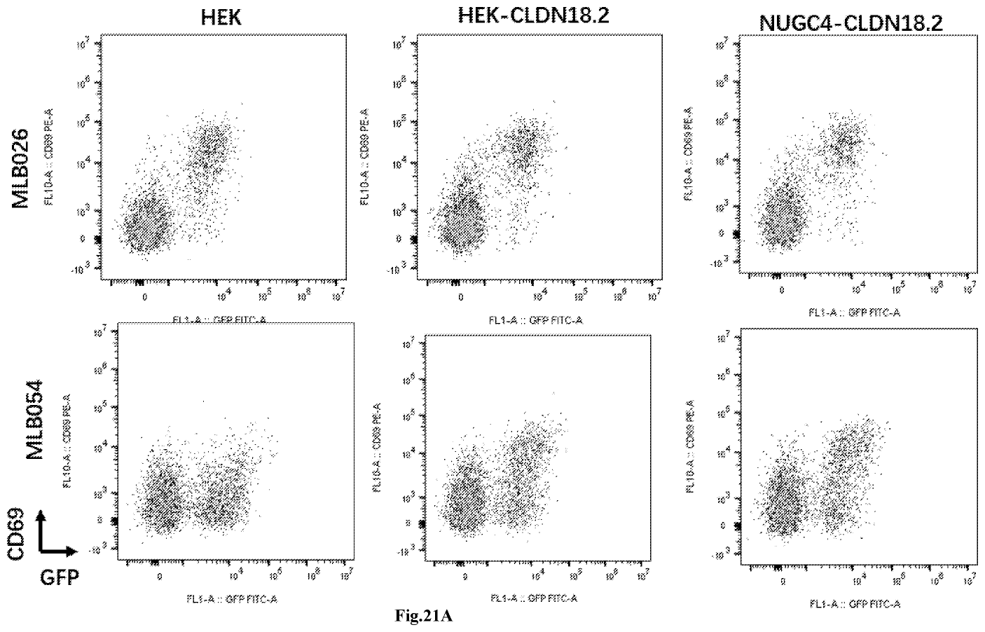

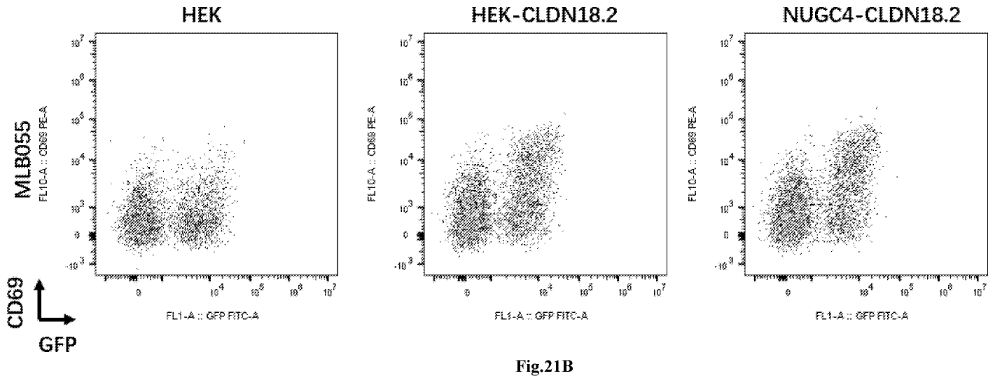

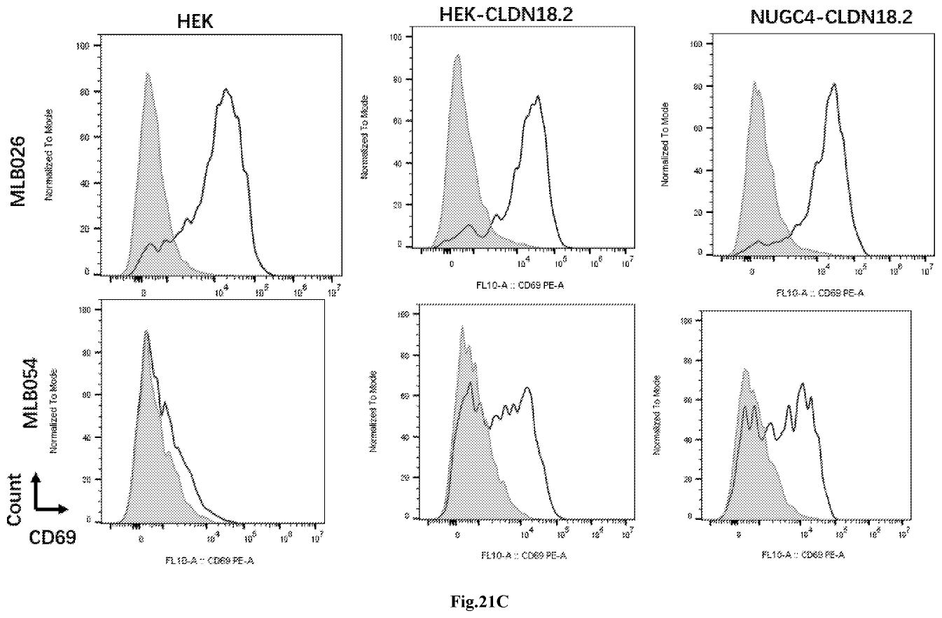

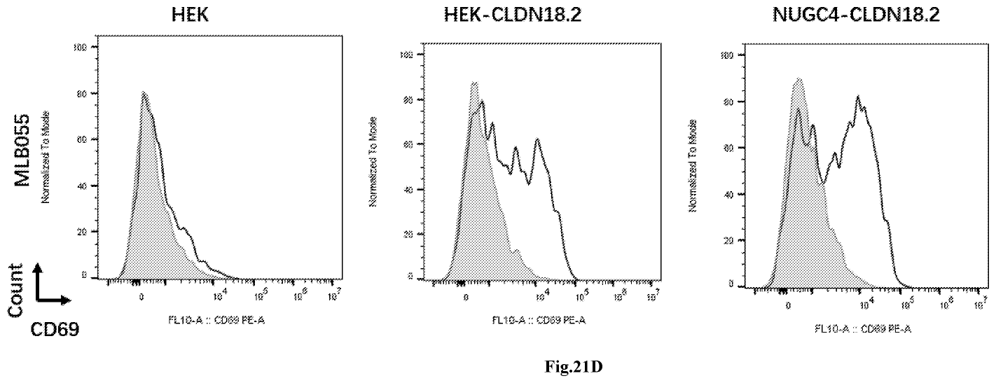

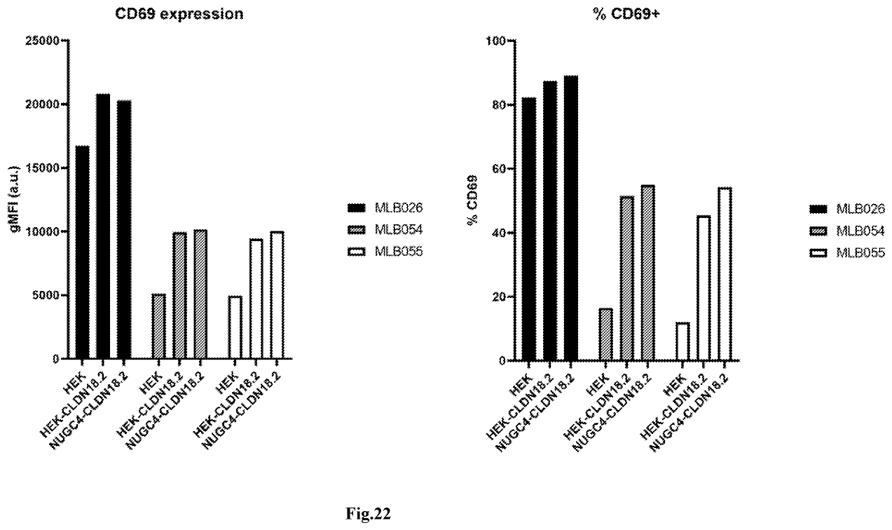

To screen for CAR T cell activity, CLDN18.2(Claudin 18.2)-specific CAR T cells were co-cultured with antigen-negative HEK293T cells or HEK293T and NUGC4 cells transduced to express CLDN18.2(Claudin 18.2). As before, 50,000 target cells were cultured at a 1:1 ratio with CAR-expressing Jurkats. As with the αCEA CAR variants (FIG. 21A-D), antigen-specific activation of the Jurkats expressing the CAR variants was observed, with decreased activation detected in the variants expressing the SEZ6L2 transmembrane domain (FIG. 22). FIG. 21A-D shows that both endocytic (MLB054, MLB055) and non-endocytic CAR (MLB026) have CD69 upregulation. FIG. 21A-B shows the CAR positive population, and FIG. 21C-D shows the overall degree of CD69 upregulation.

### Example 11. SEZ6L2-Modified CARs Used to Attenuate Cytokine Profile in T Cells Targeted Towards Claudin18.2

PBMCs were isolated via Ficoll-Paque separation and purified by CD3 negative selection to enrich the T cell population. Isolated T cells were stimulated with StemCell T cell activator (CD2/CD3/CD28) in the presence of IL-2, IL-7, and IL-15 for three days and then electroporated with hyperactive PiggyBac transposase mRNA and PiggyBac vectors MLB026 and MLB054 to induce the expression of CARs based on either the CD8a transmembrane domain or the SEZ6L2 transmembrane and juxtamembrane domain, respectively. These CAR constructs utilize the 841 scFv, which is specific to Claudin18.2.

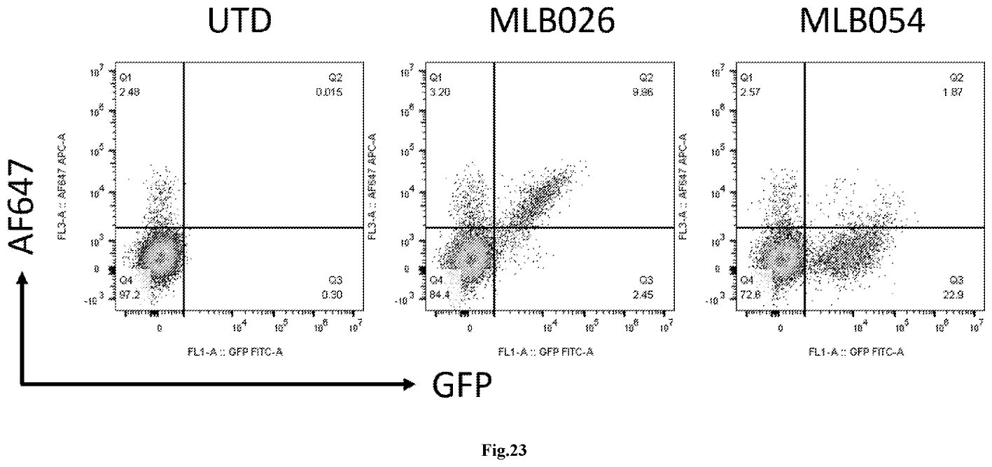

CAR surface expression was verified by flow cytometry by staining with a fluorescently-conjugated anti-human F(ab′)₂ antibody. Transduced cells were identified by the joint expression of GFP on the CAR construct via a P2A sequence. Results indicate strong surface staining in T cells expressing the CD8a transmembrane sequence (MLB026) while T cells expressing the SEZ6L2 modified CAR (MLB054) had negligible surface staining (FIG. 23).

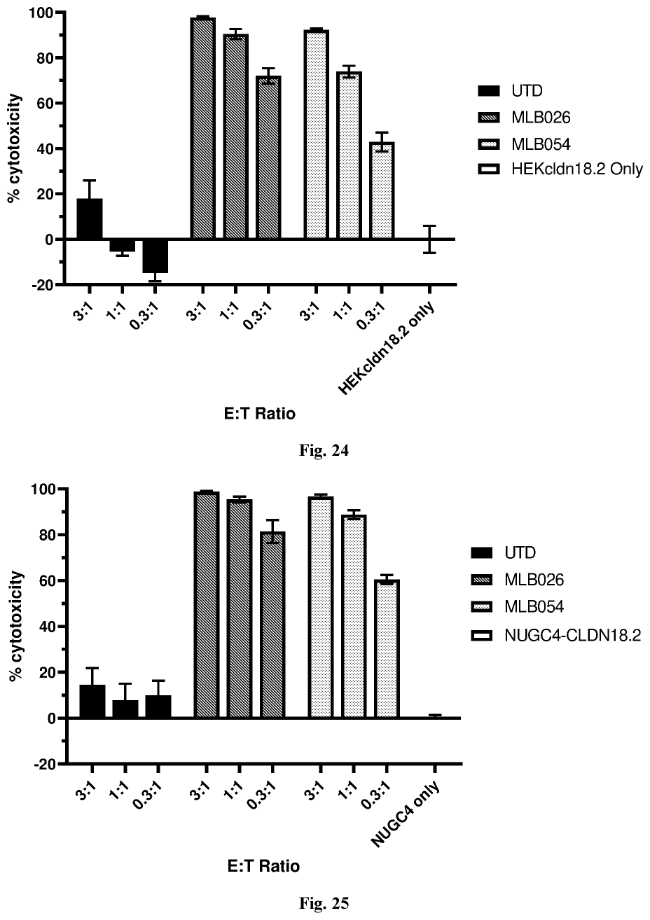

To test the functional capabilities of the anti-Claudin18.2, SEZ6L2-modified Stealth CAR, a luciferase-based cytotoxicity assay was performed against HEK293T or NUGC4 cells transduced to overexpress the Claudin18.2 isoform as well as luciferase. The number of CAR positive cells was evaluated by flow cytometry and counts were normalized to the number of CAR positive cells (FIG. 23). HEK-cldn18.2 Luc or NUGC4-cldn18.2 Luc cells were seeded at a density of 10,000 cells/well in 100 μL of ImmunoCult media in opaque 96-well plates. CAR T cells or untransduced control cells were then added at the following Effector:Target (E:T) ratios, 3:1, 1:1, and 0.3:1 in an additional 100 μL of media such that the total reaction volume was 200 L. Cells were then co-cultured for 24 hours at 37° C., 5% CO₂. At the end of this period, 100 μL media was harvested via centrifugation for cytokine analysis and NeoLite substrate was added to each well to determine the rate of live cells. Percent specific cell lysis was determined as the decrease in luminescence signal intensity in the treatment groups relative to an untreated control group. Target cells treated with either MLB026 or MLB054 showed decreased luminescence, indicative of increased cytotoxicity, relative to cells treated with untransduced T cells. Moreover, there was a decreasing dose response with decreasing E:T ratio. This trend was observed for both HEK-cldn18.2 (FIG. 24) and NUGC4-cldn18.2 (FIG. 25). This data indicates that the addition of the SEZ6L2 internalization motif and transmembrane domain to the anti-Claudin18.2 CAR construct does not diminish the cytotoxic potential of the CAR.

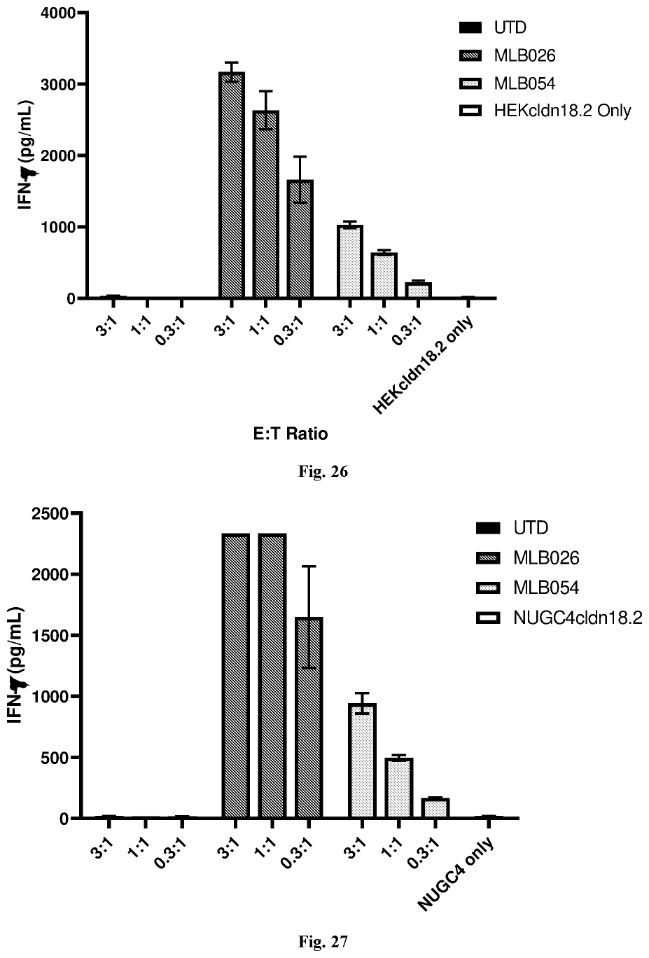

The co-culture supernatants were analyzed for expression of IFN-γ produced by the CAR T cells via sandwich ELISA. Supernatant was diluted 1-in-3 in ELISA diluent and colorimetric activity of the HRP detection substrate indicated that the SEZ6L2-modified Stealth CAR (MLB054) had a reduced cytokine secretion profile against both the HEK-cldn18.2 (FIG. 26) and NUGC4-cldn18.2 (FIG. 27) cell lines. These data indicate that the antigen recognition and cytotoxic potential of these cells is retained, while generating a lower inflammatory response. We anticipate that this may be desirable in addressing the issue of cytokine release syndrome (CRS) seen with existing CAR technologies.

### Example 12. SEZ6L2- and IL2Rβ-Modified CARs (MLB038, MLB039, and MLB080) Used to Attenuate Cytokine Profile in T Cells Targeted Toward HER2

In this Example 12, CAR constructs were designed as follows:
(1) CAR construct comprising a IL2Rβ transmembrane domain, a IL2Rβ juxtamembrane domain, and further comprising a IL2Rβ degradation sequence (DT):
(a) HER2 scFv-CD8Hinge-IL2Rβ tm jm DT-4-1BB-CD3ζ-P2A-GFP (MLB038);
(b) HER2 scFv-CD8Hinge-IL2Rβ tm jm DT-CD28-CD3ζ-P2A-GFP (MLB039);
(2) CAR construct comprising a SEZ6L2 transmembrane domain and a SEZ6L2 juxtamembrane domain:
(a) HER2 scFv-CD8Hinge-SEZ6L2 tm jm-4-1BB-CD3ζ-P2A-GFP (MLB080).
(3) The dual CAR constructs:
(a) 841 Claudin18.2 scFv-CD8Hinge-CD8 tm-4-1BB-CD3ζ-P2A-HER2 scFv-IL2Rβ tm jm DT-CD28-CD3ζ (MLB040)
(b) 841 Claudin 18.2 scFv-CD8Hinge-CD8 tm-4-1BB-CD3ζ-P2A-HER2 scFv-SEZ6L2 tm jm-CD3ζ (MLB108).
(4) The control CAR construct using CD8 transmembrane domain:
(a) HER2 scFv-CD8Hinge-CD8 tm-4-1BB-CD3ζ-P2A-GFP(MLB079).

TABLE 3
The sequences used in Examples 12-13

| | | |
|---|---|---|
| DESCRIPTION | | SEQ |
| (CAR construct) | SEQUENCE | ID NO: |

| | | |
|---|---|---|
| HER2 scFv- | MALPVTALLLPLALLLHAARPDIQMTQSPSSLSASVGDRVTITCRA | 21 |
| CD8Hinge-IL2Rβ | SQDVNTAVAWYQQKPGKAPKLLIYSASFLYSGVPSRFSGSRSGTD | |
| tm jm DT-4-1BB- | FTLTISSLQPEDFATYYCQQHYTTPPTFGQGTKVEIKGGGGSGGGG | |
| CD3ζ-P2A-GFP | SGGGGSGGGGSEVQLVESGGGLVQPGGSLRLSCAASGFNIKDTYI | |
| (MLB038) | HWVRQAPGKGLEWVARIYPTNGYTRYADSVKGRFTISADTSKNT | |
| | AYLQMNSLRAEDTAVYYCSRWGGDGFYAMDYWGQGTLVTVSST | |
| | TTPAPRPPTPAPTIASQPLSLRPEACRPAAGGAVHTRGLDFACDIPW | |
| | LGHLLVGLSGAFGFIILVYLLINCRNTGPWLKKVLKCNTPDPSKFF | |
| | SQLKRGRKKLLYIFKQPFMRPVQTTQEEDGCSCRFPEEEEGGCELR | |
| | VKFSRSADAPAYKQGQNQLYNELNLGRREEYDVLDKRRGRDPEM | |
| | GGKPRRKNPQEGLYNELQKDKMAEAYSEIGMKGERRRGKGHDG | |
| | LYQGLSTATKDTYDALHMQALPPRATNFSLLKQAGDVEENPGPM | |
| | VSKGEELFTGVVPILVELDGDVNGHKFSVSGEGEGDATYGKLTLK | |
| | FICTTGKLPVPWPTLVTTLTYGVQCFSRYPDHMKQHDFFKSAMPE | |
| | GYVQERTIFFKDDGNYKTRAEVKFEGDTLVNRIELKGIDFKEDGNI | |
| | LGHKLEYNYNSHNVYIMADKQKNGIKVNFKIRHNIEDGSVQLADH | |
| | YQQNTPIGDGPVLLPDNHYLSTQSALSKDPNEKRDHMVLLEFVTA | |
| | AGITLGMDELYK* | |
| HER2 scFv- | MALPVTALLLPLALLLHAARPDIQMTQSPSSLSASVGDRVTITCRA | 22 |
| CD8Hinge-IL2Rβ | SQDVNTAVAWYQQKPGKAPKLLIYSASFLYSGVPSRFSGSRSGTD | |
| tm jm DT- CD28- | FTLTISSLQPEDFATYYCQQHYTTPPTFGQGTKVEIKGGGGSGGGG | |
| CD3ζ-P2A-GFP | SGGGGSGGGGSEVQLVESGGGLVQPGGSLRLSCAASGFNIKDTYI | |
| (MLB039) | HWVRQAPGKGLEWVARIYPTNGYTRYADSVKGRFTISADTSKNT | |
| | AYLQMNSLRAEDTAVYYCSRWGGDGFYAMDYWGQGTLVTVSST | |
| | TTPAPRPPTPAPTIASQPLSLRPEACRPAAGGAVHTRGLDFACDIPW | |
| | LGHLLVGLSGAFGFIILVYLLINCRNTGPWLKKVLKCNTPDPSKFF | |
| | SQLRSKRSRLLHSDYMNMTPRRPGPTRKHYQPYAPPRDFAAYRSR | |
| | VKFSRSADAPAYKQGQNQLYNELNLGRREEYDVLDKRRGRDPEM | |
| | GGKPRRKNPQEGLYNELQKDKMAEAYSEIGMKGERRRGKGHDG | |
| | LYQGLSTATKDTYDALHMQALPPRATNFSLLKQAGDVEENPGPM | |
| | VSKGEELFTGVVPILVELDGDVNGHKFSVSGEGEGDATYGKLTLK | |
| | FICTTGKLPVPWPTLVTTLTYGVQCFSRYPDHMKQHDFFKSAMPE | |
| | GYVQERTIFFKDDGNYKTRAEVKFEGDTLVNRIELKGIDFKEDGNI | |
| | LGHKLEYNYNSHNVYIMADKQKNGIKVNFKIRHNIEDGSVQLADH | |
| | YQQNTPIGDGPVLLPDNHYLSTQSALSKDPNEKRDHMVLLEFVTA | |
| | AGITLGMDELYK* | |
| HER2 scFv- | MALPVTALLLPLALLLHAARPDIQMTQSPSSLSASVGDRVTITCRA | 23 |
| CD8Hinge-CD8 | SQDVNTAVAWYQQKPGKAPKLLIYSASFLYSGVPSRFSGSRSGTD | |
| tm-4-1BB-CD3ζ- | FTLTISSLQPEDFATYYCQQHYTTPPTFGQGTKVEIKGGGGSGGGG | |
| P2A-GFP | SGGGGSGGGGSEVQLVESGGGLVQPGGSLRLSCAASGFNIKDTYI | |
| (MLB079) | HWVRQAPGKGLEWVARIYPTNGYTRYADSVKGRFTISADTSKNT | |
| | AYLQMNSLRAEDTAVYYCSRWGGDGFYAMDYWGQGTLVTVSST | |
| | TTPAPRPPTPAPTIASQPLSLRPEACRPAAGGAVHTRGLDFACDIYI | |
| | WAPLAGTCGVLLLSLVITLYCKRGRKKLLYIFKQPFMRPVQTTQEE | |
| | DGCSCRFPEEEEGGCELRVKFSRSADAPAYKQGQNQLYNELNLGR | |
| | REEYDVLDKRRGRDPEMGGKPRRKNPQEGLYNELQKDKMAEAY | |
| | SEIGMKGERRRGKGHDGLYQGLSTATKDTYDALHMQALPPRGSG | |
| | ATNFSLLKQAGDVEENPGPMVSKGEELFTGVVPILVELDGDVNGH | |
| | KFSVSGEGEGDATYGKLTLKFICTTGKLPVPWPTLVTTLTYGVQCF | |
| | SRYPDHMKQHDFFKSAMPEGYVQERTIFFKDDGNYKTRAEVKFE | |
| | GDTLVNRIELKGIDFKEDGNILGHKLEYNYNSHNVYIMADKQKNG | |
| | IKVNFKIRHNIEDGSVQLADHYQQNTPIGDGPVLLPDNHYLSTQSA | |
| | LSKDPNEKRDHMVLLEFVTAAGITLGMDELYK* | |
| HER2 scFv- | MALPVTALLLPLALLLHAARPDIQMTQSPSSLSASVGDRVTITCRA | 24 |
| CD8Hinge- | SQDVNTAVAWYQQKPGKAPKLLIYSASFLYSGVPSRFSGSRSGTD | |
| SEZ6L2 tm jm-4- | FTLTISSLQPEDFATYYCQQHYTTPPTFGQGTKVEIKGGGGSGGGG | |
| 1BB-CD3ζ-P2A- | SGGGGSGGGGSEVQLVESGGGLVQPGGSLRLSCAASGFNIKDTYI | |
| GFP | HWVRQAPGKGLEWVARIYPTNGYTRYADSVKGRFTISADTSKNT | |
| (MLB080) | AYLQMNSLRAEDTAVYYCSRWGGDGFYAMDYWGQGTLVTVSST | |
| | TTPAPRPPTPAPTIASQPLSLRPEACRPAAGGAVHTRGLDFACDLAL | |
| | AILLPLGLVIVLGSGVYIYYTKLQGKSLFGFSGSHSYSPITVESDFSN | |
| | PLYKRGRKKLLYIFKQPFMRPVQTTQEEDGCSCRFPEEEEGGCELR | |
| | VKFSRSADAPAYKQGQNQLYNELNLGRREEYDVLDKRRGRDPEM | |
| | GGKPRRKNPQEGLYNELQKDKMAEAYSEIGMKGERRRGKGHDG | |
| | LYQGLSTATKDTYDALHMQALPPRGSGATNFSLLKQAGDVEENP | |
| | GPMVSKGEELFTGVVPILVELDGDVNGHKFSVSGEGEGDATYGKL | |
| | TLKFICTTGKLPVPWPTLVTTLTYGVQCFSRYPDHMKQHDFFKSA | |
| | MPEGYVQERTIFFKDDGNYKTRAEVKFEGDTLVNRIELKGIDFKED | |
| | GNILGHKLEYNYNSHNVYIMADKQKNGIKVNFKIRHNIEDGSVQL | |
| | ADHYQQNTPIGDGPVLLPDNHYLSTQSALSKDPNEKRDHMVLLEF | |
| | VTAAGITLGMDELYK* | |
| 841 Claudin18.2 | MALPVTALLLPLALLLHAARPQVQLVQSGAEVKKPGSSVKVSCKA | 25 |
| scFv-CD8Hinge- | SGGTFSSYAISWVRQAPGQGLEWMGRIIPILGIANYAQKFQGRVTV | |
| CD8 tm-4-1BB- | TADKSTSTAYMELSSLRSEDTAVYYCARERDNWDPYYYYYGMD | |
| CD3ζ-P2A-HER2 | VWGQGTTVTVSSGGGGSGGGGSGGGGSDIQMTQSPSTLSASVGD | |
| scFv-IL2Rβ tm jm | RVTITCRASQSINSWLAWYQQKPGKAPKLLIYKASSLESGVPSRFS | |
| DT-CD28-CD3ζ | GSGSGTEFTLTISSLQPDDFATYYCQQYNSFPLTFGGGTKVEIKRTT | |
| (MLB040) | TTPAPRPPTPAPTIASQPLSLRPEACRPAAGGAVHTRGLDFACDIYI | |
| | WAPLAGTCGVLLLSLVITLYCKRGRKKLLYIFKQPFMRPVQTTQEE | |
| | DGCSCRFPEEEEGGCELRVKFSRSADAPAYKQGQNQLYNELNLGR | |
| | REEYDVLDKRRGRDPEMGGKPRRKNPQEGLYNELQKDKMAEAY | |
| | SEIGMKGERRRGKGHDGLYQGLSTATKDTYDALHMQALPPRGSG | |
| | ATNFSLLKQAGDVEENPGPMALPVTALLLPLALLLHAARPDIQMT | |
| | QSPSSLSASVGDRVTITCRASQDVNTAVAWYQQKPGKAPKLLIYS | |
| | ASFLYSGVPSRFSGSRSGTDFTLTISSLQPEDFATYYCQQHYTTPPT | |
| | FGQGTKVEIKGGGGSGGGGSGGGGSGGGGSEVQLVESGGGLVQP | |
| | GGSLRLSCAASGFNIKDTYIHWVRQAPGKGLEWVARIYPTNGYTR | |
| | YADSVKGRFTISADTSKNTAYLQMNSLRAEDTAVYYCSRWGGDG | |
| | FYAMDYWGQGTLVTVSSTTTPAPRPPTPAPTIASQPLSLRPEACRP | |
| | AAGGAVHTRGLDFACDIPWLGHLLVGLSGAFGFIILVYLLINCRNT | |
| | GPWLKKVLKCNTPDPSKFFSQLRSKRSRLLHSDYMNMTPRRPGPT | |
| | RKHYQPYAPPRDFAAYRSRVKFSRSADAPAYKQGQNQLYNELNL | |
| | GRREEYDVLDKRRGRDPEMGGKPRRKNPQEGLYNELQKDKMAE | |
| | AYSEIGMKGERRRGKGHDGLYQGLSTATKDTYDALHMQALPPR* | |
| 841 Claudin 18.2 | MALPVTALLLPLALLLHAARPQVQLVQSGAEVKKPGSSVKVSCKA | 26 |
| scFv-CD8Hinge- | SGGTFSSYAISWVRQAPGQGLEWMGRIIPILGIANYAQKFQGRVTV | |
| CD8 tm-4-1BB- | TADKSTSTAYMELSSLRSEDTAVYYCARERDNWDPYYYYYGMD | |
| CD3ζ-P2A-HER2 | VWGQGTTVTVSSGGGGSGGGGSGGGGSDIQMTQSPSTLSASVGD | |
| scFv-SEZ6L2 tm | RVTITCRASQSINSWLAWYQQKPGKAPKLLIYKASSLESGVPSRFS | |
| jm-CD3ζ | GSGSGTEFTLTISSLQPDDFATYYCQQYNSFPLTFGGGTKVEIKRTT | |
| (MLB108) | TTPAPRPPTPAPTIASQPLSLRPEACRPAAGGAVHTRGLDFACDIYI | |
| | WAPLAGTCGVLLLSLVITLYCKRGRKKLLYIFKQPFMRPVQTTQEE | |
| | DGCSCRFPEEEEGGCELRVKFSRSADAPAYKQGQNQLYNELNLGR | |
| | REEYDVLDKRRGRDPEMGGKPRRKNPQEGLYNELQKDKMAEAY | |
| | SEIGMKGERRRGKGHDGLYQGLSTATKDTYDALHMQALPPRGSG | |
| | ATNFSLLKQAGDVEENPGPMALPVTALLLPLALLLHAARPDIQMT | |
| | QSPSSLSASVGDRVTITCRASQDVNTAVAWYQQKPGKAPKLLIYS | |
| | ASFLYSGVPSRFSGSRSGTDFTLTISSLQPEDFATYYCQQHYTTPPT | |
| | FGQGTKVEIKGGGGSGGGGSGGGGSGGGGSEVQLVESGGGLVQP | |
| | GGSLRLSCAASGFNIKDTYIHWVRQAPGKGLEWVARIYPTNGYTR | |
| | YADSVKGRFTISADTSKNTAYLQMNSLRAEDTAVYYCSRWGGDG | |
| | FYAMDYWGQGTLVTVSSTTTPAPRPPTPAPTIASQPLSLRPEACRP | |
| | AAGGAVHTRGLDFACDLALAILLPLGLVIVLGSGVYIYYTKLQGK | |
| | SLFGFSGSHSYSPITVESDFSNPLYRVKFSRSADAPAYKQGQNQLY | |
| | NELNLGRREEYDVLDKRRGRDPEMGGKPRRKNPQEGLYNELQKD | |
| | KMAEAYSEIGMKGERRRGKGHDGLYQGLSTATKDTYDALHMQA | |
| | LPPR* | |

Internalization Motif is bold and underlined.

TABLE 4
The sequences used in the CAR and antibodies of application

| | | |
|---|---|---|
| transmembrane | IPWLGHLLVGLSGAFGFIILVYLLINCRNTGPWLKKVLKCNTPD | 27 |
| linking | | |
| juxtamembrane of | | |
| IL2Rβ | | |
| (IL2Rβ tm jm ) | | |
| transmembrane | ALSIVLPIVLLVFLCLGVFLLWKNWRLKNINSINFDNPVY | 28 |
| linking | | |
| juxtamembrane of | | |
| LDLR | | |
| (LDLR tm jm) | | |
| transmembrane | LALAILLPLGLVIVLGSGVYIYYTKLQGKSLFGFSGSHSYSPITVESD | 29 |
| linking | FSNPLY | |
| juxtamembrane of | | |
| SEZ6L | | |
| (SEZ6L2 tm | | |
| jm) | | |
| 841 Claudin 18.2 | QVQLVQSGAEVKKPGSSVKVSCKASGGTFSSYAISWVRQAPGQGL | 30 |
| scFv | EWMGRIIPILGIANYAQKFQGRVTVTADKSTSTAYMELSSLRSEDT | |
| | AVYYCARERDNWDPYYYYYGMDVWGQGTTVTVSSGGGGSGGG | |
| | GSGGGGSDIQMTQSPSTLSASVGDRVTITCRASQSINSWLAWYQQ | |
| | KPGKAPKLLIYKASSLESGVPSRFSGSGSGTEFTLTISSLQPDDFATY | |
| | YCQQYNSFPLTFGGGTKVEIKRT | |
| HER2 scFv | DIQMTQSPSSLSASVGDRVTITCRASQDVNTAVAWYQQKPGKAPK | 31 |
| | LLIYSASFLYSGVPSRFSGSRSGTDFTLTISSLQPEDFATYYCQQHYT | |
| | TPPTFGQGTKVEIKGGGGSGGGGSGGGGSGGGGSEVQLVESGGGL | |
| | VQPGGSLRLSCAASGFNIKDTYIHWVRQAPGKGLEWVARIYPTNG | |
| | YTRYADSVKGRFTISADTSKNTAYLQMNSLRAEDTAVYYCSRWG | |
| | GDGFYAMDYWGQGTLVTVSS | |
| PD-L1 scFv | EVQLVQSGGGVVQPGRSLRLSCAASGFTFSNYAMHWVRQAPGKG | 32 |
| | LEWVAIITYAGSNEYYADSVKGRFTISRDNSKNTLYLQMNSLRPED | |
| | TAVYYCARDRIWVDYWGQGTLVTVSSGGGGSGGGGSGGGGSDIQ | |
| | MTQSPDSLAVSLGERATINCKSSQSVLYSSNNKNYLAWYQQKPGQ | |
| | PPKLLIYWASTRESGVPDRFSGSGSGTDFTLTISSLQAEDVAVYYC | |
| | QQYYSTPLTFGGGTKVEIK | |
| HA | YPYDVPDYA | 33 |
| CD8 Hinge | TTTPAPRPPTPAPTIASQPLSLRPEACRPAAGGAVHTRGLDFACD | 34 |
| CD3ζ | RVKFSRSADAPAYKQGQNQLYNELNLGRREEYDVLDKRRGRDPE | 35 |
| | MGGKPRRKNPQEGLYNELQKDKMAEAYSEIGMKGERRRGKGHD | |
| | GLYQGLSTATKDTYDALHMQALPPR | |
| 4-1BB | KRGRKKLLYIFKQPFMRPVQTTQEEDGCSCRFPEEEEGGCEL | 36 |
| CD28 | RSKRSRLLHSDYMNMTPRRPGPTRKHYQPYAPPRDFAAYRS | 37 |
| transmembrane of | ALSIVLPIVLLVFLCLGVFLLW | 38 |
| LDLR | | |
| (LDLR tm) | | |
| juxtamembrane of | KNWRLKNINSINFDNPVY | 39 |
| LDLR | | |
| (LDLR jm) | | |
| transmembrane of | LALAILLPLGLVIVLGSGVYI | 40 |
| SEZ6L2 | | |
| (SEZ6L2 tm) | | |
| juxtamembrane of | YYTKLQGKSLFGFSGSHSYSPITVESDFSNPLY | 41 |
| SEZ6L2 | | |
| (SEZ6L2 jm) | | |
| CD8 | IYIWAPLAGTCGVLLLSLVITLYC | 42 |
| transmembrane | | |
| (CD8 tm) | | |
| P2A-1 | GSGATNFSLLKQAGDVEENPGP | 43 |
| P2A-2 | ATNFSLLKQAGDVEENPGP | 44 |
| GFP | MVSKGEELFTGVVPILVELDGDVNGHKFSVSGEGEGDATYGKLTL | 45 |
| | KFICTTGKLPVPWPTLVTTLTYGVQCFSRYPDHMKQHDFFKSAMP | |
| | EGYVQERTIFFKDDGNYKTRAEVKFEGDTLVNRIELKGIDFKEDGN | |
| | ILGHKLEYNYNSHNVYIMADKQKNGIKVNFKIRHNIEDGSVQLAD | |
| | HYQQNTPIGDGPVLLPDNHYLSTQSALSKDPNEKRDHMVLLEFVT | |
| | AAGITLGMDELYK | |
| Leader Sequence | MALPVTALLLPLALLLHAARP | 46 |
| VH of | EVQLVQSGGGVVQPGRSLRLSCAASGFTFSSYAMHWVRQAPGKG | 47 |
| hCLDN18.2-808 | LEWVAVISYDGSNKYYADSVKGRFTISRDNSKNTLYLQMNSLRAE | |
| antibody | DTAVYYCARELRFFDWLLGSAFDIWGQGTTVTVSS | |
| VL of | DIVMTQSPSSLSASVGDRVTITCRASQGIRNDLGWYQQKPGKAPK | 48 |
| hCLDN18.2-808 | LLIYAASSLQSGVPSRFSGSGSGTDFTLTISSLQPEDFATYYCLQDY | |
| antibody | NYPRTFGQGTKVEIK | |
| VH of | QVQLVQSGAEVKKPGSSVKVSCKASGGTFSSYAISWVRQAPGQGL | 49 |
| hCLDN18.2-841 | EWMGRIIPILGIANYAQKFQGRVTVTADKSTSTAYMELSSLRSEDT | |
| antibody | AVYYCARERDNWDPYYYYYGMDVWGQGTTVTVSS | |
| VL of | DIQMTQSPSTLSASVGDRVTITCRASQSINSWLAWYQQKPGKAPK | 50 |
| hCLDN18.2-841 | LLIYKASSLESGVPSRFSGSGSGTEFTLTISSLQPDDFATYYCQQYN | |
| antibody | SFPLTFGGGTKVEIK | |

PBMCs were isolated via Ficoll-Paque separation and purified by CD3 negative selection to enrich the T cell population. Isolated T cells were stimulated with StemCell T cell activator (CD2/CD3/CD28) in the presence of IL-2, IL-7, and IL-15 for three days and then electroporated with hyperactive PiggyBac transposase mRNA and PiggyBac vectors MLB038, MLB039, MLB079, and MLB080. All four CAR variants were engineered with the 4D5V8 scFv derived from the Herceptin antibody, which recognizes HER2 (the amino acid sequence of 4D5V8 Her2 scFv is SEQ ID NO. 31). The MLB038 and MLB039 CARs were designed based on the IL2 Receptor β transmembrane and juxtamembrane sequence. MLB038 and MLB039 are distinguishable by their coreceptor signaling domains, MLB038 was designed using the CD28 coreceptor domain and MLB039 was designed with the 4-11BB signaling domain. Both formats also have the CD3ζ intracellular ITAM signaling motifs. MLB079 was designed as a control and based on the CD8α transmembrane domain, while MLB080 was engineered with the SEZ6L2 transmembrane and juxtamembrane sequences.

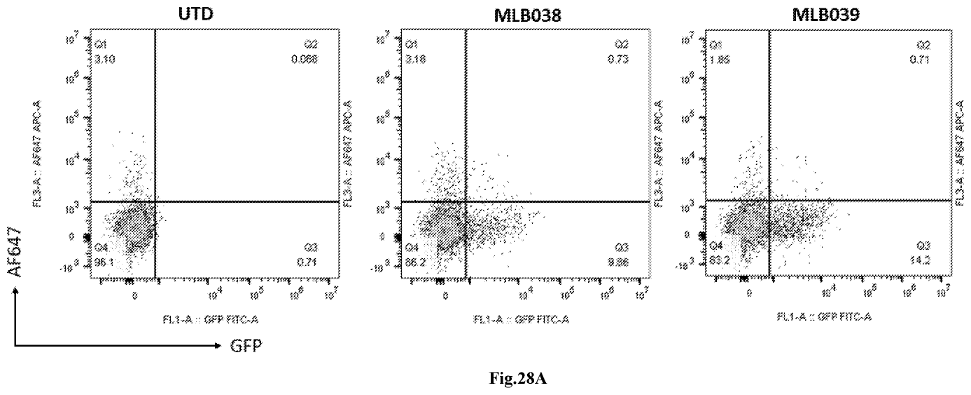

CAR surface expression was verified by flow cytometry by staining with a fluorescently-conjugated anti-human F(ab′)₂ antibody. Transduced cells were identified by the joint expression of GFP on the CAR construct via a P2A sequence. Results indicate strong surface staining in T cells expressing the CD8a transmembrane sequence while T cells expressing the IL2Rβ- or SEZ6L2-modified CARs (MLB038, MLB039, MLB080) had greatly reduced surface staining indicative of their predominantly intracellular localization (FIG. 28A-B). FIG. 28A-B shows that the control CAR of MLB079 has an increase in AF647 staining, while other endocytic CARs (MLB038, MLB039, MLB080) did not, indicating strong surface expression in the original CAR (MLB079) and undetectable surface staining in the stealth CAR (MLB038, MLB039, MLB080).

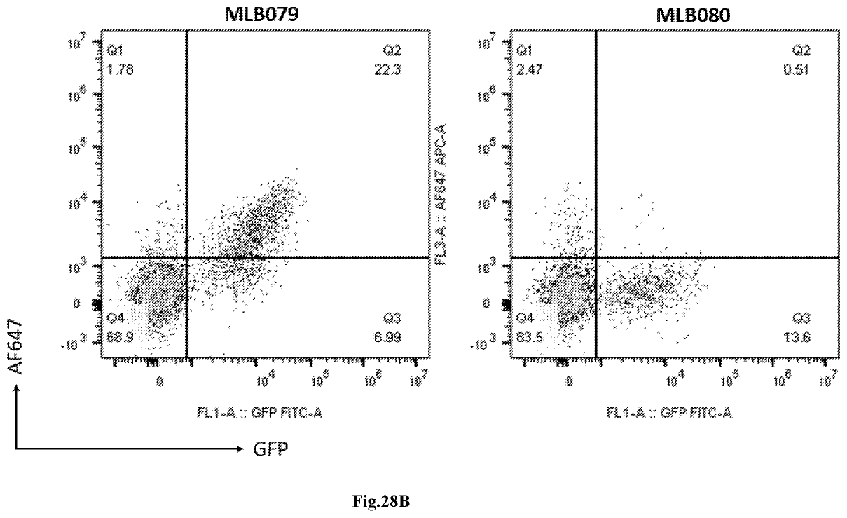

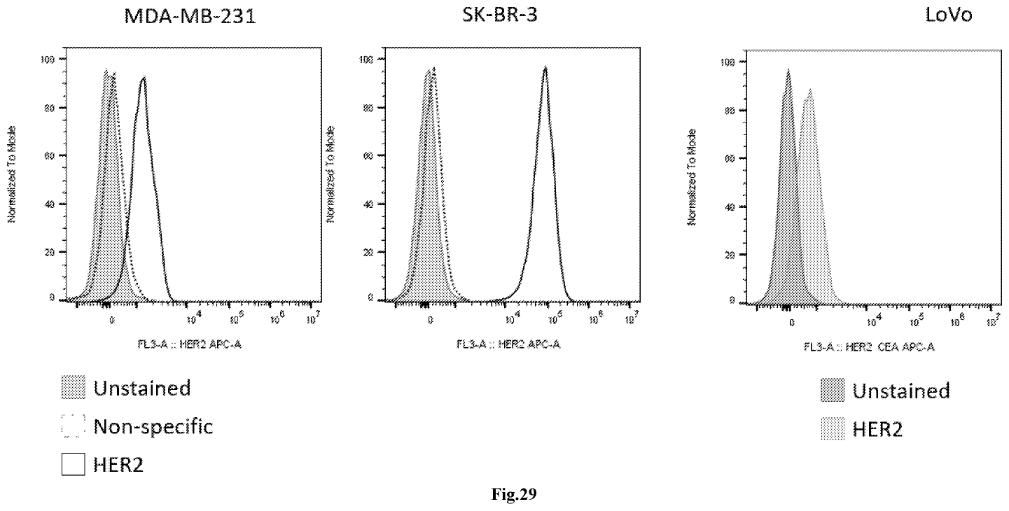

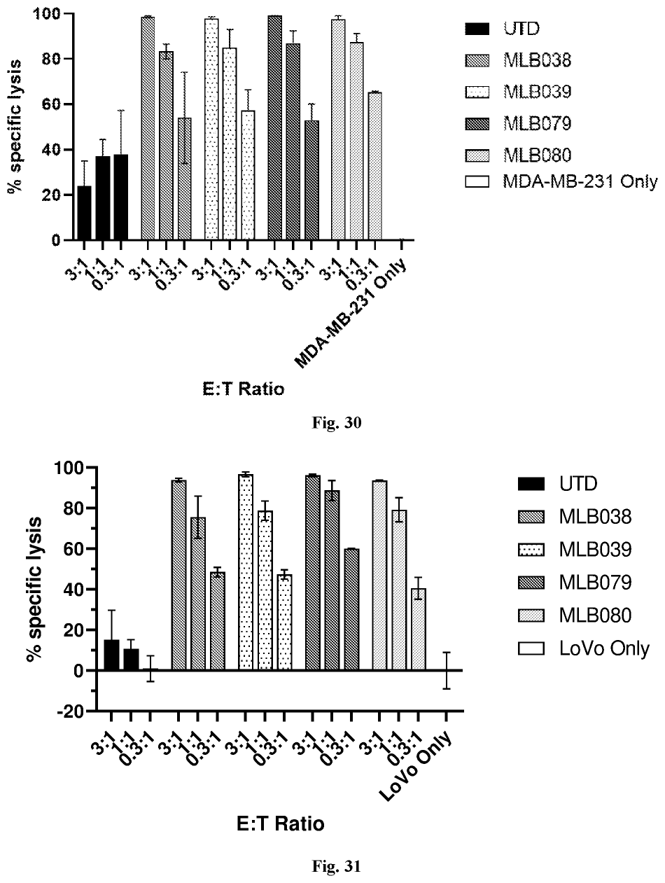

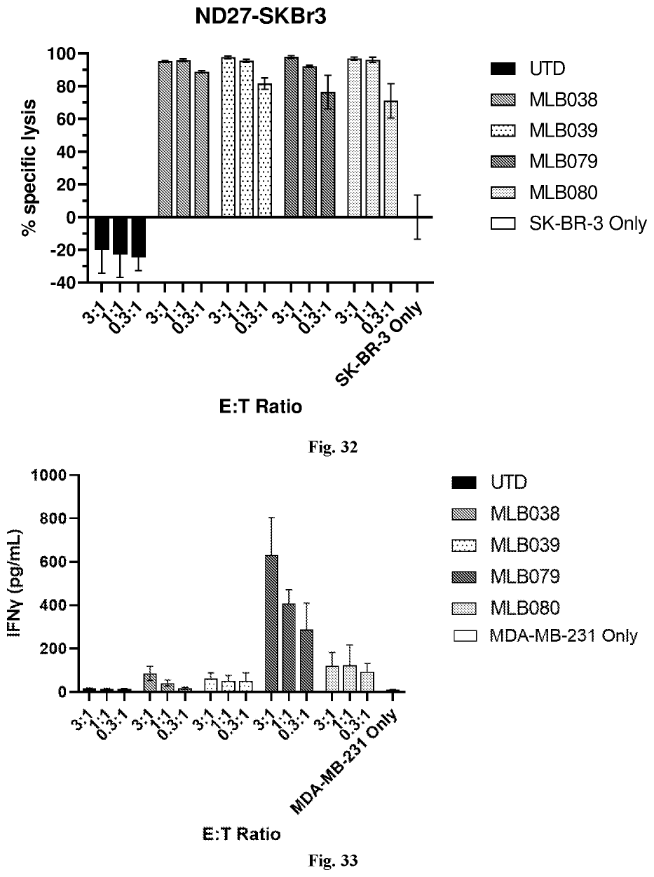

To test the functional capacity of the anti-HER2 stealth CAR variants, luciferase-based cytotoxicity assays were performed against the following cell lines SK-BR-3, LoVo, and MDA-MB-231—which is clinically described as a triple-negative breast cancer cell line (FIG. 29). FIG. 29 showed varying degrees of HER2 expression on the luciferase target cell lines. MDA-MB-231 is clinically HER2 negative, and showed low HER2 staining. LoVo cells also showed low levels of HER2 antibody staining. SK-BR-3 cells showed high levels of HER2 staining. Differences in the HER2 expression profile of the target cells allowed us to evaluate the efficacy of our HER2 CAR variants. CAR T cells or untransduced control cells were then added at the following Effector:Target (E:T) ratios, 3:1, 1:1, and 0.3:1 in an additional 100 μL of media such that the total reaction volume was 200 μL. Cells were then co-cultured for 24 hours at 37° C., 5% CO2. At the end of this period, 100 μL media was harvested via centrifugation for cytokine analysis and NeoLite substrate was added to each well to determine the rate of live cells. Percent specific cell lysis was determined as the decrease in luminescence signal intensity in the treatment groups relative to an untreated control group. Antigen-specific cell lysis was detectable against all three target cell lines: MDA-MB-231, LoVo, SKBR3, respectively (FIG. 30, FIG. 31, FIG. 32) with all four CAR variants (MLB038, MLB039, MLB079, MLB080). The cytotoxic activity of each CAR variant was broadly comparable and dose-dependent, i.e., as the E:T ratio was reduced, so was target cell killing. Comparison of variants MLB038 and MLB039 indicated that the co-receptor domain did not influence in vitro killing efficiency.

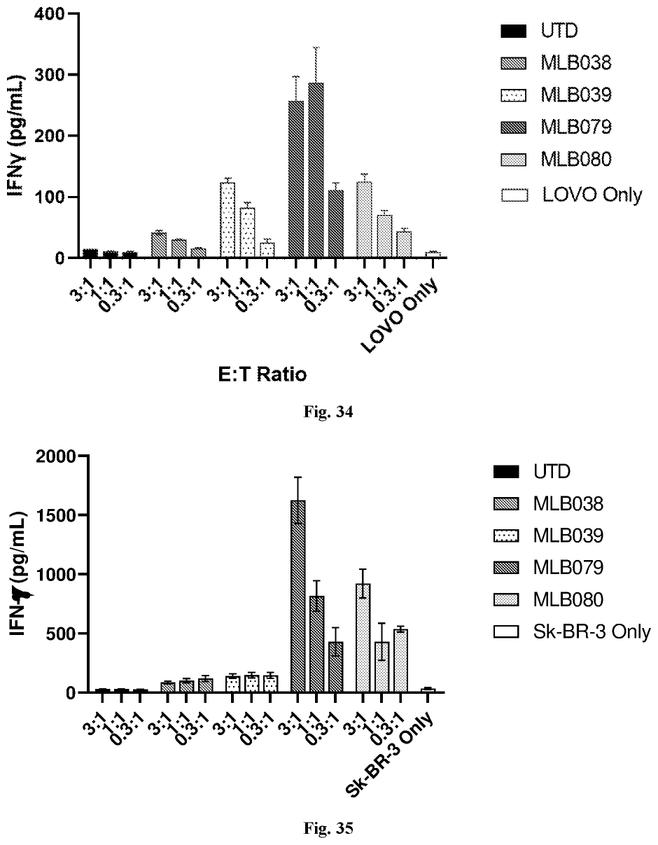

The co-culture supernatants were analyzed for expression of IFN-γ produced by the CAR T cells via sandwich ELISA. Supernatant was diluted 1-in-3 in ELISA diluent and colorimetric activity of the HRP detection substrate indicated that the Stealth CAR variants MLB038, MLB039, and MLB080 had reduced cytokine secretion relative to the CD8ca transmembrane control CAR, MLB079, against all of the cell lines tested: MDA-MB-231, LoVo, SKBR3 (FIG. 33, FIG. 34, FIG. 35). In the HER2-high expression cell line, expression of IFN-γ was higher in cells transduced to express MLB080 than both MLB038 and MLB039, but still lower than the original CAR format, MLB079 (FIG. 35). This indicates that the SEZ6L2-modified CAR is still able to retain cytokine signaling, though to a lower degree, when targeting cells with high tumor-associated antigen expression. Cytokine expression decreased across all cell lines in a dose-dependent manner with decreasing E:T ratio. These results indicated that the CAR coreceptor signaling domain did not greatly impact cytokine expression and that the Stealth CARs (MLB038, MLB039, MLB080) had reduced inflammatory cytokine profiles relative to the control CAR (MLB079).

### Example 13. Claudin18.2/HER2 Targeted Dual CARs Show Cytotoxic Potential and Enhanced Functionality Against Tumor Cell Lines Expressing One or Two Tumor-Associated Antigens

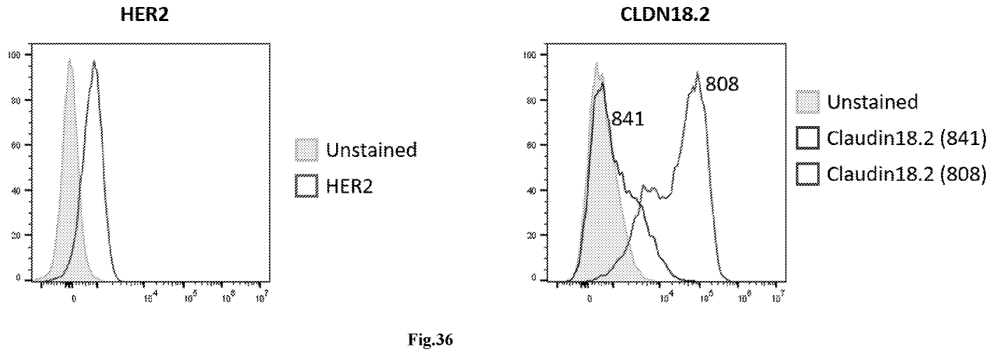

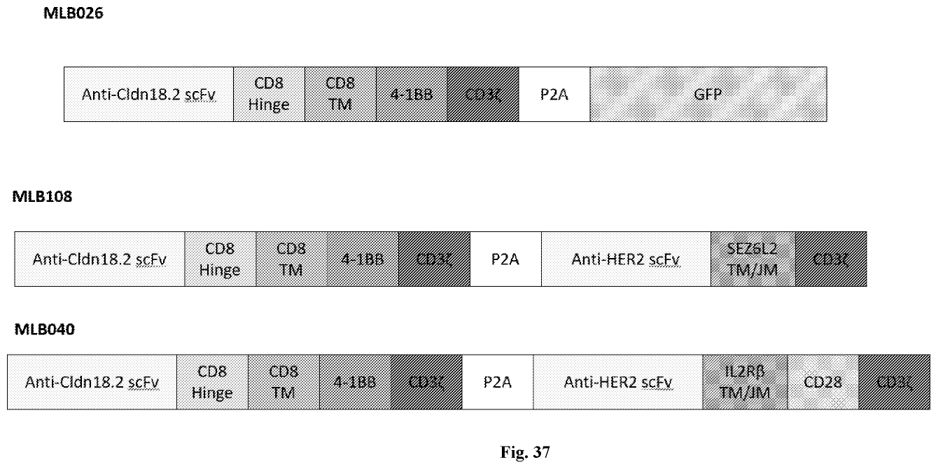

HEKcldn18.2 Cells were profiled for HER2 and Claudin18.2 expression using either a commercially available HER2 antibody or the Claudin18.2 antibody clones developed in-house, hCLDN18.2-808 antibody (the amino acid sequence of VH is SEQ ID NO:47; the amino acid sequence of VL is SEQ ID NO:48) and hCLDN18.2-841 antibody (the amino acid sequence of VH is SEQ ID NO:49; the amino acid sequence of VL is SEQ ID NO:50) (FIG. 36). The left panel of FIG. 36 shows HER2 staining. The distance between the negative peak and the positive peak is small, therefore HER2 expression is low. This data indicates strong Claudin18.2 staining, with low HER2 expression. A dual CAR was designed with a so-called “dominant CAR” based on the CD8a transmembrane domain and specific to Claudin18.2 and a “non-dominant CAR” modified with the SEZ6L2 transmembrane and juxtamembrane domain, lacking a co-receptor domain, and specific to HER2 (MLB108). An alternative non-dominant CAR was designed using the IL2Rβ transmembrane and juxtamembrane sequences, but inclusive of the CD28 co-receptor domain (MLB040) (FIG. 37). Functionality of the Dual CARs was compared to the Claudin18.2 CAR, MLB026.

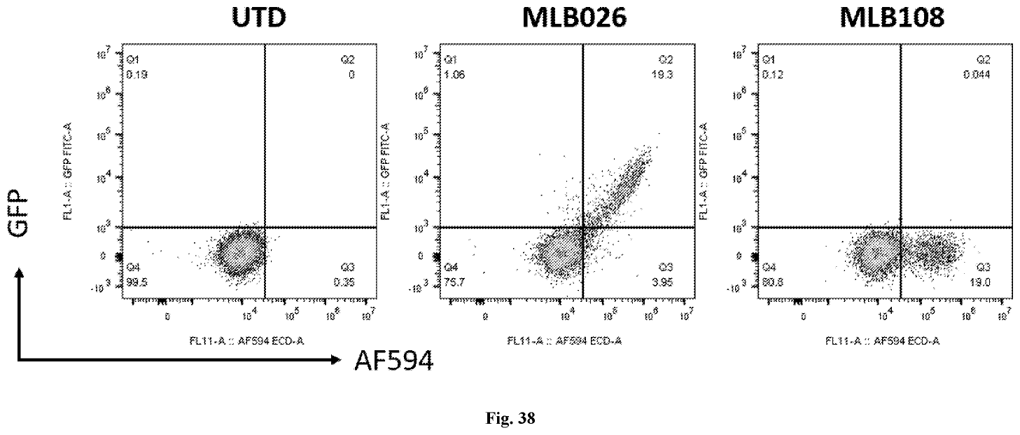

PBMCs were isolated via Ficoll-Paque separation and purified by CD3 negative selection to enrich the T cell population. Isolated T cells were stimulated with StemCell T cell activator (CD2/CD3/CD28) in the presence of IL-2, IL-7, and IL-15 for three days and then electroporated with hyperactive PiggyBac transposase mRNA and PiggyBac vectors MLB026 and MLB108. CAR surface expression was verified by flow cytometry by staining with a fluorescently-conjugated anti-human F(ab′)₂ antibody (FIG. 38). The FIG. 38 shows that MLB108 staining increases in AF594 direction compared with non-transduced cells (UTD), while MLB026 staining increases in GFP and AF594 direction. FIG. 38 shows that we are able to detect expression of the dominant CAR at the surface in MLB108.

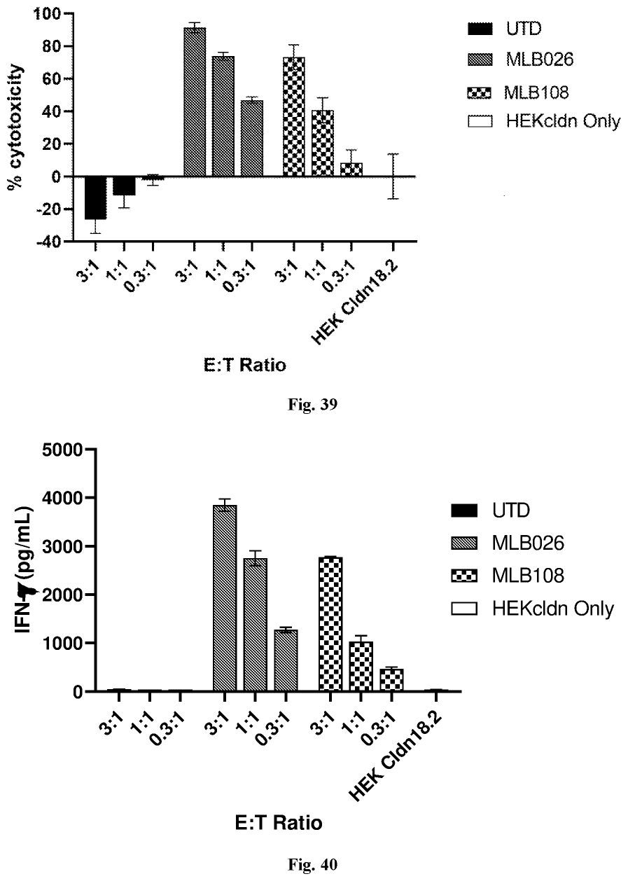

Functional assays indicated that Dual CAR MLB108 was able to elicit antigen specific killing of HEKcldn18.2 cells in a dose-dependent manner (FIG. 39). Comparison of IFN-γ release in the cytotoxicity assay supernatant indicated that there was strong IFN-γ release by cells expressing the MLB108 dual CAR, consistent with targeting of Claudin18.2 by the dominant CAR (FIG. 40).

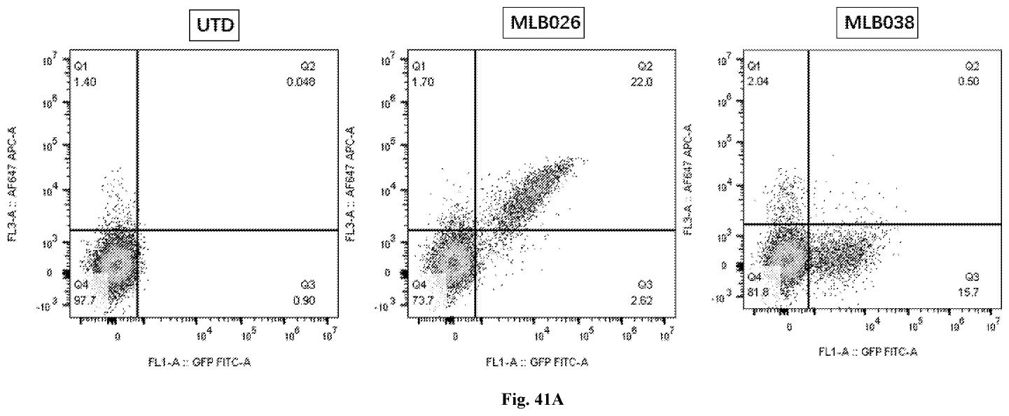

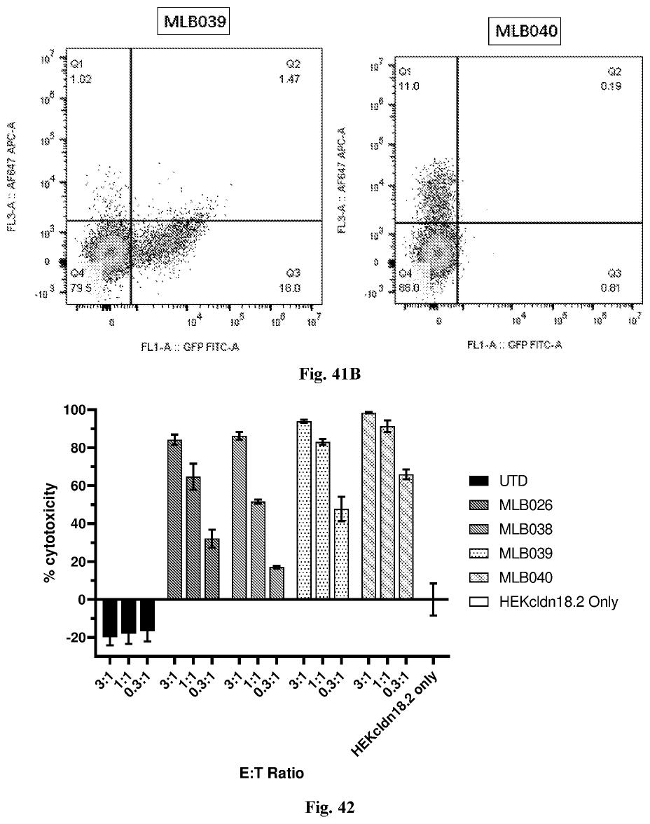

In additional dual CAR assays, the MLB040 Dual CAR format was compared with both the Claudin18.2-specific dominant CAR (MLB026) and the HER2 stealth CARs (MLB038 and MLB039). PBMCs were isolated via Ficoll-Paque separation and purified by CD3 negative selection to enrich the T cell population. Isolated T cells were stimulated with StemCell T cell activator (CD2/CD3/CD28) in the presence of IL-2, IL-7, and IL-15 for three days and then electroporated with hyperactive PiggyBac transposase mRNA and PiggyBac vectors MLB026, MLB038, MLB039, and MLB040. CAR surface expression was verified by flow cytometry by staining with a fluorescently-conjugated anti-human F(ab′)₂ antibody (FIG. 41A-B). FIG. 41A-B shows that MLB038 and MLB039 have an endocytosis effect, which are stained only in the abscissa direction, and MLB026 without endocytosis is stained in both abscissa and ordinate directions. Dual CAR MLB040 is not stained in the abscissa, but is stained in the HER2 direction of the ordinate.

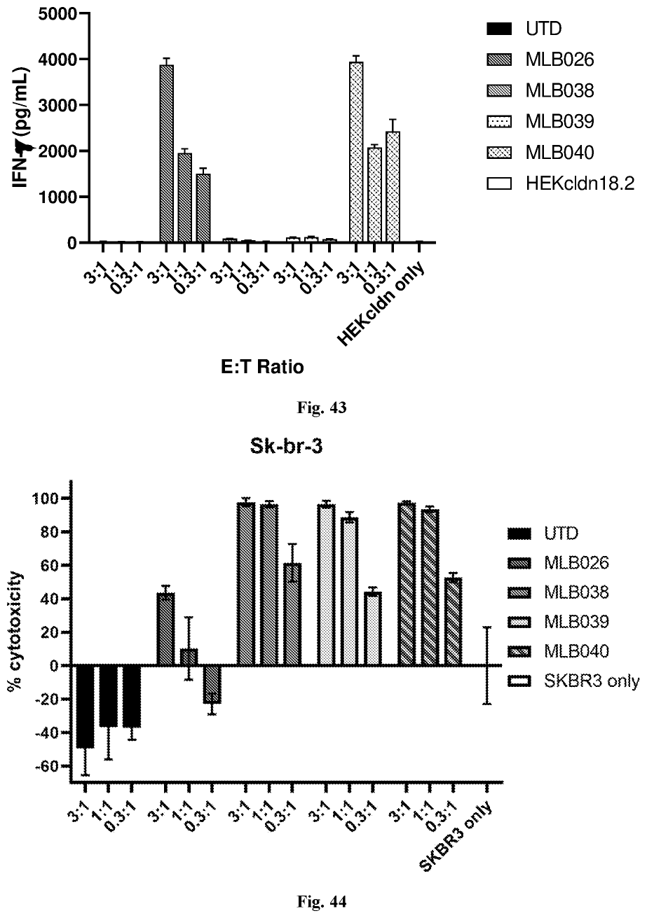

Functional assays indicated a slight enhancement of cytotoxicity against HEKcldn18.2 cells, which express high levels of Claudin18.2 and low levels of HER2 (FIG. 42). In this assay we compared the functional activity of the single Claudin18.2 (MLB026) and HER2 CAR constructs with a Claudin18.2/HER2 dual CAR (MLB040). MLB040 is comprised of a dominant claudin18.2 CAR (MLB026) and IL2Rβ Stealth CAR with CD28 co-receptor domain (MLB039), we therefore compared the activity of MLB040 with MLB026, MLB038, and MLB039 against HEK293Tcldn18.2 cells to determine the activity of the CAR constructs in isolation and in tandem. We observed strong cytotoxicity of the single CARs (MLB026, MLB038, and MLB039) against HEK293T cells which decreased with a decreasing E:T ratio showing antigen-specific lysis. Moreover, we observed enhanced target cell killing by the CLDN18.2/HER2 dual CAR (MLB040) at all of the E:T ratios tested. These results prove that the stealth CAR (MLB040) remains functional within a dual CAR system. ELISA analysis of the cytotoxicity assay supernatant showed comparable levels of IFN-γ release from both MLB026 and MLB040 consistent with targeting of Claudin18.2 by the dominant CAR. No cytokine release was detected from either of the HER2 single stealth CARs, MLB038 and MLB039 despite detectable cytotoxicity (FIG. 43). When tested against the HER2 high expression cell line, SK-BR-3, strong antigen-specific cytotoxicity was observed by T cells expressing MLB038, MLB039, and MLB040, while weak cytotoxicity consistent with background activation was observed in the MLB026 single CAR. These results indicate that the Dual Targeting CARs were able to recognize both Claudin18.2 and HER2 (FIG. 44).

### OTHER EMBODIMENTS

It is to be understood that while the disclosure has been described in conjunction with the detailed description thereof, the foregoing description is intended to illustrate and not limit the scope of the disclosure, which is defined by the scope of the appended claims. Other aspects, advantages, and modifications are within the scope of the following claims.

The various embodiments described above can be combined to provide further embodiments. All of the U.S. patents, U.S. patent application publications, U.S. patent applications, foreign patents, foreign patent applications and non-patent publications referred to in this specification and/or listed in the Application Data Sheet are incorporated herein by reference, in their entirety. Aspects of the embodiments can be modified to employ concepts of the various patents, applications and publications to provide yet further embodiments.

These and other changes can be made to the embodiments in light of the above-detailed description. In general, in the following claims, the terms used should not be construed to limit the claims to the specific embodiments disclosed in the specification and the claims, but should be construed to include all possible embodiments along with the full scope of equivalents to which such claims are entitled. Accordingly, the claims are not limited by the disclosure.

### CITATION LIST

1. Ruella, M., & Maus, M. V. (2016). Catch me if you can: Leukemia Escape after CD19-Directed T Cell Immunotherapies. In Computational and Structural Biotechnology Journal (Vol. 14, pp. 357-362). Elsevier B. V. https://doi.org/10.1016/j.csbj.2016.09.003.
2. Fousek, K., Watanabe, J., Joseph, S. K., George, A., An, X., Byrd, T. T., Morris, J. S., Luong, A., Martínez-Paniagua, M. A., Sanber, K., Navai, S. A., Gad, A. Z., Salsman, V. S., Mathew, P. R., Kim, H. N., Wagner, D. L., Brunetti, L., Jang, A., Baker, M. L., . . . Ahmed, N. (2020). CAR T-cells that target acute B-lineage leukemia irrespective of CD19 expression. Leukemia, 1, 4. https://doi.org/10.1038/s41375-020-0792-2.
3. Hémar, A., Lieb, M., Subtil, A., Disanto, J. P., & Dautry-Varsat, A. (1994). Endocytosis of the β chain of interleukin-2 receptor requires neither interleukin-2 nor the 7 chain. European Journal of Immunology, 24(9), 1951-1955. https://doi.org/10.1002/eji.1830240902.
4. Tao, W., Moore, R., Meng, Y., Smith, E. R. & Xu, X. X. Endocytic adaptors Arh and Dab2 control homeostasis of circulatory cholesterol. J. Lipid Res. 57, 809-817.
5. Fasano, T., Sun, X. M., Patel, D. D. & Soutar, A. K. Degradation of LDLR protein mediated by ‘gain of function’ PCSK9 mutants in normal and ARH cells. Atherosclerosis 203, 166-171.
6. Bonifacino, J. S. & Traub, L. M. Signals for sorting of transmembrane proteins to endosomes and lysosomes. Annual Review of Biochemistry vol. 72 395-447 (2003).

1. A chimeric antigen receptor (CAR) comprising:
(1) an extracellular ligand-binding domain comprising a single chain variable fragment (scFv) specifically binding to a predetermined antigen;
(2) a transmembrane (tm) linking juxtamembrane (jm) domain;
wherein the transmembrane linking juxtamembrane domain comprises (a) an IL2 Receptor 3 chain (IL2Rβ) transmembrane domain and an IL2Rβ juxtamembrane domain, wherein and the transmembrane linking juxtamembrane domain is adjacent to an IL2Rβ degradation sequence (DT);
(b) a Low-Density Lipoprotein Receptor (LDLR) transmembrane domain and an LDLR juxtamembrane domain; or
(c) a Seizure 6-like Protein 2 (SEZ6L2) transmembrane domain and a SEZ6L2 juxtamembrane domain; and
(3) an intracellular domain.

2. The CAR according to claim 1, wherein the CAR comprises from N-terminal to C-terminal: TAA scFv-CD8Hinge-IL2Rβ tm jm DT-4-1BB-CD3ζ, TAA scFv-CD8Hinge-IL2Rβ tm jm DT-CD28-CD3ζ, TAA scFv-CD8Hinge-LDLR tm jm-4-1BB-CD3ζ, or TAA scFv-CD8Hinge-SEZ6L2 tm jm-4-1BB-CD3ζ;
wherein preferably, the TAA scFv is selected from one or more of CEA scFv, Claudin 18.2 scFv and HER2 scFv; more preferably, CEA scFv is MN14op CEA scFv, or Claudin 18.2 scFv is 841 Claudin 18.2 scFv; most preferably, MN14op CEA scFv, 841 Claudin 18.2 scFv, or HER2 scFv comprises an amino acid sequence that is at least about 95%, 96%, 97%, 98%, 99% or 100% identical to the amino acid sequence represented by SEQ ID NO. 5, 30, or 31 respectively;
wherein preferably, the N-terminal of the CAR further contains a Leader Sequence and/or a HA Sequence.

3. The CAR according to claim 2, wherein the CAR comprises from N-terminal to C-terminal:
HA-MN14op CEA scFv-CD8Hinge-IL2Rβ tm jm DT-4-1BB-CD3ζ,
HA-MN14op CEA scFv-CD8Hinge-IL2Rβ tm jm DT-4-1BB-CD3ζ-P2A-GFP,
HER2 scFv-CD8Hinge-IL2Rβ tm jm DT-4-1BB-CD3ζ-P2A-GFP,
HER2 scFv-CD8Hinge-IL2Rβ tm jm DT-CD28-CD3ζ-P2A-GFP,
MN14op CEA scFv-CD8Hinge-IL2Rβ tm jm DT-4-1BB-CD3ζ-P2A-GFP,
MN14op CEA scFv-CD8Hinge-LDLR tm jm-4-1BB-CD3ζ-P2A-GFP,
MN14op CEA scFv-CD8Hinge-SEZ6L2 tm jm-4-1BB-CD3ζ-P2A-GFP,
HER2 scFv-CD8Hinge-SEZ6L2 tm jm-4-1BB-CD3ζ-P2A-GFP, or HER2 scFv-CD8Hinge-SEZ6L2 tm jm-4-1BB-CD3ζ-P2A-GFP, optionally the CAR does not include P2A-GFP and/or HA;
wherein preferably, the CAR comprises an amino acid sequence that is at least about 95%, 96%, 97%, 98%, 99% or 100% identical to the amino acid sequence represented by SEQ ID NO. 9, 14, 15, 17, 18, 19, 21, 22 or 24 respectively, optionally the amino acid sequence of CAR does not include the amino acid sequence of P2A-GFP, Leader Sequence, and/or HA.

4. A chimeric antigen receptor (CAR) comprising:
(1) an extracellular ligand-binding domain comprising single chain variable fragment (scFv) specifically binding to a predetermined antigen; wherein preferably, the predetermined antigen is a tumor-associated antigen (TAA); more preferably, the TAA is selected from one or more of: CEA, Claudin 18.2, CGC3, Receptor tyrosine kinase-like Orphan Receptor 1 (ROR1), CD38, CD19, CD20, CD22, BCMA, CAIX, CD446, CD13, EGFR, EGFRvIII, EpCam, GD2, EphA2, HER1, HER2, ICAM-1, IL13Ra2, Mesothelin, MUC1, MUC16, NKG2D, PSCA, NY-ESO-1, MART-1, WT1, MAGE-A10, MAGE-A3, MAGE-A4, EBV, PD1, PD-L1, CD25, IL-2, and CD3;
(2) a transmembrane domain, and
(3) a cytoplasmic segment comprising an IL2Rβ degradation sequence (DT) and at least one signaling domain; wherein preferably, the IL2Rβ degradation sequence comprises an amino acid sequence that is at least about 95%, 96%, 97%, 98%, 99% or 100% identical to the amino acid sequence represented by SEQ ID NO: 2; wherein preferably, the IL2Rβ degradation sequence is at the C-terminal of the cytoplasmic segment.

5. The CAR according to claim 4, wherein the CAR comprises from N-terminal to C-terminal: TAA scFv-CD8Hinge-CD8 tm-4-1BB-CD3ζ-IL2Rβ DT;
wherein preferably, the N-terminal of the CAR further contains Leader Sequence and/or HA Sequence;
wherein preferably, the TAA scFv is CEA scFv; more preferably, the CEA scFv is MN14op CEA scFv; most preferably, MN14op CEA scFv comprises an amino acid sequence that is at least about 95%, 96%, 97%, 98%, 99% or 100% identical to the amino acid sequence represented by SEQ ID NO. 5; most preferably, the CAR comprises an amino acid sequence that is at least about 95%, 96%, 97%, 98%, 99% or 100% identical to the amino acid sequence represented by SEQ ID NO. 8 or 16 respectively, optionally the amino acid sequence of CAR does not include the amino acid sequence of P2A-GFP, Leader Sequence, and/or HA.

6. The CAR according to claim 1, wherein the transmembrane (tm) of the IL2Rβ, LDLR, or SEZ6L2 comprises an amino acid sequence that is at least about 95%, 96%, 97%, 98%, 99% or 100% identical to the amino acid sequence represented by SEQ ID NO. 1, 38 or 40 respectively;
the juxtamembrane (m) of the IL2Rβ, LDLR, or SEZ6L2 comprises an amino acid sequence that is at least about 95%, 96%, 97%, 98%, 99% or 100% identical to the amino acid sequence represented by SEQ ID NO. 4, 39 or 41 respectively;
the transmembrane (tm) linking juxtamembrane (m) of the IL2Rβ, LDLR, or SEZ6L2 comprises an amino acid sequence that is at least about 95%, 96%, 97%, 98%, 99% or 100% identical to the amino acid sequence represented by SEQ ID NO. 27, 28, or 29 respectively;
the MN14op CEA scFv, 841 Claudin 18.2 scFv, HER2 scFv, PD-L1 scFv, HA, CD8Hinge, CD3ζ, 4-1BB, CD28, CD8 tm, GFP, or Leader Sequence comprises an amino acid sequence that is at least about 95%, 96%, 97%, 98%, 99% or 100% identical to the amino acid sequence represented by SEQ ID NO. 5, 30, 31, 32, 33, 34, 35, 36, 37, 42, 45 or 46 respectively; and/or
the P2A comprises an amino acid sequence that is at least about 95%, 96%, 97%, 98%, 99% or 100% identical to the amino acid sequence represented by SEQ ID NO. 43 or 44.

7. A dual CAR comprising: a first CAR according to claim 1, and
a second CAR comprising:
(1) an extracellular ligand-binding domain comprising scFv specifically binding to a predetermined antigen;
(2) a transmembrane domain; wherein preferably, the transmembrane domain is CD8 transmembrane domain;
(3) an intracellular domain; wherein preferably, the intracellular domain comprises signaling domain; more preferably, the signaling domain comprises one or more signaling domains selected from the group consisting of a 4-1BB signaling domain, a CD28 signaling domain and a CD3ζ signaling domain;
wherein the first CAR targets an antigen and the second CAR targets another antigen;
wherein preferably, the first CAR and the second CAR is linked by P2A; more preferably the P2A comprises an amino acid sequence that is at least about 95%, 96%, 97%, 98%, 99% or 100% identical to the amino acid sequence represented by SEQ ID NO. 43 or 44;
wherein preferably, the dual CAR comprises N-terminal to C-terminal:
TAA scFv-CD8Hinge-IL2Rβ tm jm DT-4-1BB-CD3ζ-P2A-another TAA scFv-CD8Hinge-CD8 tm-CD28-CD3ζ, TAA scFv-CD8Hinge-CD8 tm-4-1BB-CD3ζ-P2A-another TAA scFv-CD8Hinge-IL2Rβ tm jm DT-CD28-CD3ζ, TAA scFv-CD8Hinge-LDLR tm jm-4-1BB-CD3ζ-P2A-another TAA scFv-CD8Hinge-CD8 tm-CD28-CD3ζ, TAA scFv-CD8Hinge-CD8 tm-4-1BB-CD3ζ-P2A-another TAA scFv-CD8Hinge-LDLR tm jm-CD28-CD3ζ, TAA scFv-CD8Hinge-SEZ6L2 tm jm-4-1BB-CD3ζ-P2A-another TAA scFv-CD8Hinge-CD8 tm-CD28, or TAA scFv-CD8Hinge-CD8 tm-4-1BB-CD3ζ-P2A-another TAA scFv-CD8Hinge-SEZ6L2 tm jm-CD3ζ.

8. The dual CAR according to claim 7, wherein the dual CAR comprises N-terminal to C-terminal: 841 Claudin 18.2 scFv-CD8Hinge-SEZ6L2 tm jm-4-1BB-CD3ζ-P2A-PD-L1 scFv-CD8Hinge-CD8 tm-CD28-(G₄S)₂-GFP, 841 Claudin18.2 scFv-CD8Hinge-CD8 tm-4-1BB-CD3ζ-P2A-HER2 scFv-IL2Rβ tm jm DT-CD28-CD3ζ, or 841 Claudin18.2 scFv-CD8Hinge-CD8 tm-4-1BB-CD3ζ-P2A-HER2 scFv-SEZ6L2 tm jm-CD3ζ respectively, optionally the dual CAR does not include (G₄S)₂-GFP; more preferably, 841 Claudin 18.2 scFv, HER2 scFv, or PD-L1 scFv comprises an amino acid sequence that is at least about 95%, 96%, 97%, 98%, 99% or 100% identical to the amino acid sequence represented by SEQ ID NO. 30, 31, or 32 respectively; most preferably, the CAR comprises an amino acid sequence that is at least about 95%, 96%, 97%, 98%, 99% or 100% identical to the amino acid sequence represented by SEQ ID NO. 20, 25 or 26 respectively, optionally the amino acid sequence of CAR does not include the amino acid sequence of (G₄S)₂-GFP and/or Leader Sequence.

9. A nucleic acid comprising a polynucleotide encoding the CAR of claim 1.

10. A vector comprising the nucleic acid of claim 9.

11. A composition comprising the CAR of claim 1.

12. A method of treating disease in a subject in need thereof, comprising administering to the subject an effective amount of the composition of claim 11;
wherein preferably, the disease is cancer; more preferably, the cancer is selected from one or more of hematological malignancy or solid tumor; most preferably, the solid tumor is ovarian cancer, pancreatic cancer, colon cancer, colorectal cancer, lymphoma, esophageal cancer, lung cancer, hepatic cancer, head-neck cancer, or cancer of the gallbladder.

13. A method of reducing the cytotoxicity of a CAR-T cell towards normal cells, using the CAR of claim 1.

14. A method of producing a CAR-T cell with reduced cytotoxicity towards normal cells comprising:
(1) introducing to a host cell the nucleic acid of claim 9, and
(2) isolating and/or expanding the CAR-T cells following the introduction.

15. The CAR of claim 1, wherein the predetermined antigen is a tumor-associated antigen (TAA).

16. The CAR of claim 15, wherein the TAA is selected from one or more of: CEA, Claudin 18.2, CGC3, Receptor tyrosine kinase-like Orphan Receptor 1 (ROR1), CD38, CD19, CD20, CD22, BCMA, CAIX, CD446, CD13, EGFR, EGFRvIII, EpCam, GD2, EphA2, HER1, HER2, ICAM-1, IL13Ra2, Mesothelin, MUC1, MUC16, PSCA, NY-ESO-1, MART-1, WT1, MAGE-A10, MAGE-A3, MAGE-A4, EBV, NKG2D, PD1, PD-L1, CD25, HL-2, and CD3.

17. The CAR of claim 1, wherein the IL2R degradation sequence is at the C-terminal of the transmembrane linking juxtamembrane domain.

18. The CAR of claim 1, wherein the IL2Rβ degradation sequence comprises an amino acid sequence that is at least about 95%, 96%, 97%, 98%, 99% or 100% identical to the amino acid sequence represented by SEQ ID NO: 2.

19. The CAR of claim 1, wherein the intracellular domain comprises a signaling domain.

20. The CAR of claim 19, wherein the signaling domain comprises one or more signaling domains selected from the group consisting of a 4-1BB signaling domain, a CD28 signaling domain and a CD3ζ signaling domain.


SEQUENCE LISTING (50 sequences)

SEQ ID NO: 1 - protein, 25 aa, Artificial Sequence
  IL2Rbeta transmembrane domain (tm)
IPWLGHLLVGLSGAFGFIILVYLLI

SEQ ID NO: 2 - protein, 8 aa, Artificial Sequence
  IL2Rbeta degradation sequence (DT)
PSKFFSQL

SEQ ID NO: 3 - protein, 27 aa, Artificial Sequence
  IL2Rbeta truncated cytoplasmic domain
NCRNTGPWLKKVLKCNTPDPSKFFSQL

SEQ ID NO: 4 - protein, 19 aa, Artificial Sequence
  IL2Rbeta juxtamembrane sequence (jm)
NCRNTGPWLKKVLKCNTPD

SEQ ID NO: 5 - protein, 246 aa, Artificial Sequence
  MN14op CEA scFv
EVQLVESGGGVVKPGGSLRLSCSASGFDFTTYWMSWVRQAPGKGLEWIGEIHPDSSTINYAPSLKDRFTISRDNAKNTLYLQMDSLRPEDTGVYYCASLYFGFPWFAYWGQGTTVTVSSGGGGSGGGGSGGGGSGGGGSDIVLTQSPSSLSASLGDRVTITCKASQDVGTSVAWYQQKPGKAPKLLIYWTSTRHTGVPSRFSGSGSGTDFTFTISSLQPEDIATYYCQQYSLYRSFGGGTKVEIKG

SEQ ID NO: 6 - protein, 490 aa, Artificial Sequence
  MN14op CEA scFv-CD8Hinge-CD8tm-4-1BB-CD3zeta (LBC001)
MALPVTALLLPLALLLHAARPEVQLVESGGGVVKPGGSLRLSCSASGFDFTTYWMSWVRQAPGKGLEWIGEIHPDSSTINYAPSLKDRFTISRDNAKNTLYLQMDSLRPEDTGVYYCASLYFGFPWFAYWGQGTTVTVSSGGGGSGGGGSGGGGSGGGGSDIVLTQSPSSLSASLGDRVTITCKASQDVGTSVAWYQQKPGKAPKLLIYWTSTRHTGVPSRFSGSGSGTDFTFTISSLQPEDIATYYCQQYSLYRSFGGGTKVEIKGTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 7 - protein, 499 aa, Artificial Sequence
  HA-MN14op CEA scFv-CD8Hinge-CD8tm-4-1BB-CD3zeta (MLB001)
MALPVTALLLPLALLLHAARPYPYDVPDYAEVQLVESGGGVVKPGGSLRLSCSASGFDFTTYWMSWVRQAPGKGLEWIGEIHPDSSTINYAPSLKDRFTISRDNAKNTLYLQMDSLRPEDTGVYYCASLYFGFPWFAYWGQGTTVTVSSGGGGSGGGGSGGGGSGGGGSDIVLTQSPSSLSASLGDRVTITCKASQDVGTSVAWYQQKPGKAPKLLIYWTSTRHTGVPSRFSGSGSGTDFTFTISSLQPEDIATYYCQQYSLYRSFGGGTKVEIKGTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 8 - protein, 507 aa, Artificial Sequence
  HA-MN14ops cFv-CD8Hinge-CD8 tm-4-1BB-CD3zeta-DT (MLB002)
MALPVTALLLPLALLLHAARPYPYDVPDYAEVQLVESGGGVVKPGGSLRLSCSASGFDFTTYWMSWVRQAPGKGLEWIGEIHPDSSTINYAPSLKDRFTISRDNAKNTLYLQMDSLRPEDTGVYYCASLYFGFPWFAYWGQGTTVTVSSGGGGSGGGGSGGGGSGGGGSDIVLTQSPSSLSASLGDRVTITCKASQDVGTSVAWYQQKPGKAPKLLIYWTSTRHTGVPSRFSGSGSGTDFTFTISSLQPEDIATYYCQQYSLYRSFGGGTKVEIKGTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRPSKFFSQL

SEQ ID NO: 9 - protein, 527 aa, Artificial Sequence
  HA-MN14op CEA scFv-CD8Hinge-IL2Rbeta tm jm DT- 4-1BB-CD3zeta (MLB003)
MALPVTALLLPLALLLHAARPYPYDVPDYAEVQLVESGGGVVKPGGSLRLSCSASGFDFTTYWMSWVRQAPGKGLEWIGEIHPDSSTINYAPSLKDRFTISRDNAKNTLYLQMDSLRPEDTGVYYCASLYFGFPWFAYWGQGTTVTVSSGGGGSGGGGSGGGGSGGGGSDIVLTQSPSSLSASLGDRVTITCKASQDVGTSVAWYQQKPGKAPKLLIYWTSTRHTGVPSRFSGSGSGTDFTFTISSLQPEDIATYYCQQYSLYRSFGGGTKVEIKGTTTPAPRPPTPAPTIASQPLSLRPEACRPAAGGAVHTRGLDFACDIPWLGHLLVGLSGAFGFIILVYLLINCRNTGPWLKKVLKCNTPDPSKFFSQLKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 10 - protein, 231 aa, Artificial Sequence
  CAR-DTdeltascFv
TTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRPSKFFSQL

SEQ ID NO: 11 - protein, 251 aa, Artificial Sequence
  CAR- IL2Rbeta TMdeltascFv
TTTPAPRPPTPAPTIASQPLSLRPEACRPAAGGAVHTRGLDFACDIPWLGHLLVGLSGAFGFIILVYLLINCRNTGPWLKKVLKCNTPDPSKFFSQLKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 12 - protein, 751 aa, Artificial Sequence
  MN14op CEA scFv -CD8Hinge-CD8 tm-4-1BB-CD3zeta- P2A-GFP (MLB010)
MALPVTALLLPLALLLHAARPEVQLVESGGGVVKPGGSLRLSCSASGFDFTTYWMSWVRQAPGKGLEWIGEIHPDSSTINYAPSLKDRFTISRDNAKNTLYLQMDSLRPEDTGVYYCASLYFGFPWFAYWGQGTTVTVSSGGGGSGGGGSGGGGSGGGGSDIVLTQSPSSLSASLGDRVTITCKASQDVGTSVAWYQQKPGKAPKLLIYWTSTRHTGVPSRFSGSGSGTDFTFTISSLQPEDIATYYCQQYSLYRSFGGGTKVEIKGTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRGSGATNFSLLKQAGDVEENPGPMVSKGEELFTGVVPILVELDGDVNGHKFSVSGEGEGDATYGKLTLKFICTTGKLPVPWPTLVTTLTYGVQCFSRYPDHMKQHDFFKSAMPEGYVQERTIFFKDDGNYKTRAEVKFEGDTLVNRIELKGIDFKEDGNILGHKLEYNYNSHNVYIMADKQKNGIKVNFKIRHNIEDGSVQLADHYQQNTPIGDGPVLLPDNHYLSTQSALSKDPNEKRDHMVLLEFVTAAGITLGMDELYK

SEQ ID NO: 13 - protein, 754 aa, Artificial Sequence
  841 Claudin 18.2 scFv-CD8Hinge-CD8 tm-4-1BB- CD3zeta-P2A-GFP (MLB026)
MALPVTALLLPLALLLHAARPQVQLVQSGAEVKKPGSSVKVSCKASGGTFSSYAISWVRQAPGQGLEWMGRIIPILGIANYAQKFQGRVTVTADKSTSTAYMELSSLRSEDTAVYYCARERDNWDPYYYYYGMDVWGQGTTVTVSSGGGGSGGGGSGGGGSDIQMTQSPSTLSASVGDRVTITCRASQSINSWLAWYQQKPGKAPKLLIYKASSLESGVPSRFSGSGSGTEFTLTISSLQPDDFATYYCQQYNSFPLTFGGGTKVEIKRTTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRGSGATNFSLLKQAGDVEENPGPMVSKGEELFTGVVPILVELDGDVNGHKFSVSGEGEGDATYGKLTLKFICTTGKLPVPWPTLVTTLTYGVQCFSRYPDHMKQHDFFKSAMPEGYVQERTIFFKDDGNYKTRAEVKFEGDTLVNRIELKGIDFKEDGNILGHKLEYNYNSHNVYIMADKQKNGIKVNFKIRHNIEDGSVQLADHYQQNTPIGDGPVLLPDNHYLSTQSALSKDPNEKRDHMVLLEFVTAAGITLGMDELYK

SEQ ID NO: 14 - protein, 779 aa, Artificial Sequence
  MN14op CEA scFv-CD8Hinge-IL2Rbeta tm jm DT-4- 1BB-CD3zeta-P2A-GFP (MLB020)
MALPVTALLLPLALLLHAARPEVQLVESGGGVVKPGGSLRLSCSASGFDFTTYWMSWVRQAPGKGLEWIGEIHPDSSTINYAPSLKDRFTISRDNAKNTLYLQMDSLRPEDTGVYYCASLYFGFPWFAYWGQGTTVTVSSGGGGSGGGGSGGGGSGGGGSDIVLTQSPSSLSASLGDRVTITCKASQDVGTSVAWYQQKPGKAPKLLIYWTSTRHTGVPSRFSGSGSGTDFTFTISSLQPEDIATYYCQQYSLYRSFGGGTKVEIKGTTTPAPRPPTPAPTIASQPLSLRPEACRPAAGGAVHTRGLDFACDIPWLGHLLVGLSGAFGFIILVYLLINCRNTGPWLKKVLKCNTPDPSKFFSQLKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRGSGATNFSLLKQAGDVEENPGPMVSKGEELFTGVVPILVELDGDVNGHKFSVSGEGEGDATYGKLTLKFICTTGKLPVPWPTLVTTLTYGVQCFSRYPDHMKQHDFFKSAMPEGYVQERTIFFKDDGNYKTRAEVKFEGDTLVNRIELKGIDFKEDGNILGHKLEYNYNSHNVYIMADKQKNGIKVNFKIRHNIEDGSVQLADHYQQNTPIGDGPVLLPDNHYLSTQSALSKDPNEKRDHMVLLEFVTAAGITLGMDELYK

SEQ ID NO: 15 - protein, 786 aa, Artificial Sequence
  HA-MN14op CEA scFv-CD8Hinge-IL2Rbeta tm jm DT- 4-1BB-CD3zeta-P2A-GFP (MLB013)
MALPVTALLLPLALLLHAARPYPYDVPDYAMEVQLVESGGGVVKPGGSLRLSCSASGFDFTTYWMSWVRQAPGKGLEWIGEIHPDSSTINYAPSLKDRFTISRDNAKNTLYLQMDSLRPEDTGVYYCASLYFGFPWFAYWGQGTTVTVSSGGGGSGGGGSGGGGSGGGGSDIVLTQSPSSLSASLGDRVTITCKASQDVGTSVAWYQQKPGKAPKLLIYWTSTRHTGVPSRFSGSGSGTDFTFTISSLQPEDIATYYCQQYSLYRSFGGGTKVEIKGTTTPAPRPPTPAPTIASQPLSLRPEACRPAAGGAVHTRGLDFACDIPWLGHLLVGLSGAFGFIILVYLLINCRNTGPWLKKVLKCNTPDPSKFFSQLKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRATNFSLLKQAGDVEENPGPMVSKGEELFTGVVPILVELDGDVNGHKFSVSGEGEGDATYGKLTLKFICTTGKLPVPWPTLVTTLTYGVQCFSRYPDHMKQHDFFKSAMPEGYVQERTIFFKDDGNYKTRAEVKFEGDTLVNRIELKGIDFKEDGNILGHKLEYNYNSHNVYIMADKQKNGIKVNFKIRHNIEDGSVQLADHYQQNTPIGDGPVLLPDNHYLSTQSALSKDPNEKRDHMVLLEFVTAAGITLGMDELYK

SEQ ID NO: 16 - protein, 759 aa, Artificial Sequence
  MN14op CEA scFv-CD8Hinge- CD8 tm-4-1BB-CD3zeta- IL2Rbeta DT-P2A-GFP (MLB025)
MALPVTALLLPLALLLHAARPEVQLVESGGGVVKPGGSLRLSCSASGFDFTTYWMSWVRQAPGKGLEWIGEIHPDSSTINYAPSLKDRFTISRDNAKNTLYLQMDSLRPEDTGVYYCASLYFGFPWFAYWGQGTTVTVSSGGGGSGGGGSGGGGSGGGGSDIVLTQSPSSLSASLGDRVTITCKASQDVGTSVAWYQQKPGKAPKLLIYWTSTRHTGVPSRFSGSGSGTDFTFTISSLQPEDIATYYCQQYSLYRSFGGGTKVEIKGTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRPSKFFSQLGSGATNFSLLKQAGDVEENPGPMVSKGEELFTGVVPILVELDGDVNGHKFSVSGEGEGDATYGKLTLKFICTTGKLPVPWPTLVTTLTYGVQCFSRYPDHMKQHDFFKSAMPEGYVQERTIFFKDDGNYKTRAEVKFEGDTLVNRIELKGIDFKEDGNILGHKLEYNYNSHNVYIMADKQKNGIKVNFKIRHNIEDGSVQLADHYQQNTPIGDGPVLLPDNHYLSTQSALSKDPNEKRDHMVLLEFVTAAGITLGMDELYK

SEQ ID NO: 17 - protein, 781 aa, Artificial Sequence
  MN14op CEA scFv-CD8Hinge-SEZ6L2 tm jm-4-1BB- CD3zeta-P2A-GFP (MLB047)
MALPVTALLLPLALLLHAARPEVQLVESGGGVVKPGGSLRLSCSASGFDFTTYWMSWVRQAPGKGLEWIGEIHPDSSTINYAPSLKDRFTISRDNAKNTLYLQMDSLRPEDTGVYYCASLYFGFPWFAYWGQGTTVTVSSGGGGSGGGGSGGGGSGGGGSDIVLTQSPSSLSASLGDRVTITCKASQDVGTSVAWYQQKPGKAPKLLIYWTSTRHTGVPSRFSGSGSGTDFTFTISSLQPEDIATYYCQQYSLYRSFGGGTKVEIKGTTTPAPRPPTPAPTIASQPLSLRPEACRPAAGGAVHTRGLDFACDLALAILLPLGLVIVLGSGVYIYYTKLQGKSLFGFSGSHSYSPITVESDFSNPLYKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRGSGATNFSLLKQAGDVEENPGPMVSKGEELFTGVVPILVELDGDVNGHKFSVSGEGEGDATYGKLTLKFICTTGKLPVPWPTLVTTLTYGVQCFSRYPDHMKQHDFFKSAMPEGYVQERTIFFKDDGNYKTRAEVKFEGDTLVNRIELKGIDFKEDGNILGHKLEYNYNSHNVYIMADKQKNGIKVNFKIRHNIEDGSVQLADHYQQNTPIGDGPVLLPDNHYLSTQSALSKDPNEKRDHMVLLEFVTAAGITLGMDELYK

SEQ ID NO: 18 - protein, 767 aa, Artificial Sequence
  MN14op CEA scFv-CD8Hinge-LDLR tm jm-4-1BB- CD3zeta-P2A-GFP (MLB048)
MALPVTALLLPLALLLHAARPEVQLVESGGGVVKPGGSLRLSCSASGFDFTTYWMSWVRQAPGKGLEWIGEIHPDSSTINYAPSLKDRFTISRDNAKNTLYLQMDSLRPEDTGVYYCASLYFGFPWFAYWGQGTTVTVSSGGGGSGGGGSGGGGSGGGGSDIVLTQSPSSLSASLGDRVTITCKASQDVGTSVAWYQQKPGKAPKLLIYWTSTRHTGVPSRFSGSGSGTDFTFTISSLQPEDIATYYCQQYSLYRSFGGGTKVEIKGTTTPAPRPPTPAPTIASQPLSLRPEACRPAAGGAVHTRGLDFACDALSIVLPIVLLVFLCLGVFLLWKNWRLKNINSINFDNPVYKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRGSGATNFSLLKQAGDVEENPGPMVSKGEELFTGVVPILVELDGDVNGHKFSVSGEGEGDATYGKLTLKFICTTGKLPVPWPTLVTTLTYGVQCFSRYPDHMKQHDFFKSAMPEGYVQERTIFFKDDGNYKTRAEVKFEGDTLVNRIELKGIDFKEDGNILGHKLEYNYNSHNVYIMADKQKNGIKVNFKIRHNIEDGSVQLADHYQQNTPIGDGPVLLPDNHYLSTQSALSKDPNEKRDHMVLLEFVTAAGITLGMDELYK

SEQ ID NO: 19 - protein, 784 aa, Artificial Sequence
  841 Claudin 18.2 scFv-CD8Hinge-SEZ6L2 tm jm-4- 1BB-CD3zeta-P2A-GFP (MLB054)
MALPVTALLLPLALLLHAARPQVQLVQSGAEVKKPGSSVKVSCKASGGTFSSYAISWVRQAPGQGLEWMGRIIPILGIANYAQKFQGRVTVTADKSTSTAYMELSSLRSEDTAVYYCARERDNWDPYYYYYGMDVWGQGTTVTVSSGGGGSGGGGSGGGGSDIQMTQSPSTLSASVGDRVTITCRASQSINSWLAWYQQKPGKAPKLLIYKASSLESGVPSRFSGSGSGTEFTLTISSLQPDDFATYYCQQYNSFPLTFGGGTKVEIKRTTTTPAPRPPTPAPTIASQPLSLRPEACRPAAGGAVHTRGLDFACDLALAILLPLGLVIVLGSGVYIYYTKLQGKSLFGFSGSHSYSPITVESDFSNPLYKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRGSGATNFSLLKQAGDVEENPGPMVSKGEELFTGVVPILVELDGDVNGHKFSVSGEGEGDATYGKLTLKFICTTGKLPVPWPTLVTTLTYGVQCFSRYPDHMKQHDFFKSAMPEGYVQERTIFFKDDGNYKTRAEVKFEGDTLVNRIELKGIDFKEDGNILGHKLEYNYNSHNVYIMADKQKNGIKVNFKIRHNIEDGSVQLADHYQQNTPIGDGPVLLPDNHYLSTQSALSKDPNEKRDHMVLLEFVTAAGITLGMDELYK

SEQ ID NO: 20 - protein, 1169 aa, Artificial Sequence
  841 Claudin 18.2 scFv-CD8Hinge-SEZ6L2 tm jm-4- 1BB-CD3zeta-P2A-PD-L1 scFv-CD8Hinge-CD8 tm-CD28-(G4S)2-GFP (MLB055)
MALPVTALLLPLALLLHAARPQVQLVQSGAEVKKPGSSVKVSCKASGGTFSSYAISWVRQAPGQGLEWMGRIIPILGIANYAQKFQGRVTVTADKSTSTAYMELSSLRSEDTAVYYCARERDNWDPYYYYYGMDVWGQGTTVTVSSGGGGSGGGGSGGGGSDIQMTQSPSTLSASVGDRVTITCRASQSINSWLAWYQQKPGKAPKLLIYKASSLESGVPSRFSGSGSGTEFTLTISSLQPDDFATYYCQQYNSFPLTFGGGTKVEIKRTTTTPAPRPPTPAPTIASQPLSLRPEACRPAAGGAVHTRGLDFACDLALAILLPLGLVIVLGSGVYIYYTKLQGKSLFGFSGSHSYSPITVESDFSNPLYKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRGSGATNFSLLKQAGDVEENPGPMALPVTALLLPLALLLHAARPEVQLVQSGGGVVQPGRSLRLSCAASGFTFSNYAMHWVRQAPGKGLEWVAIITYAGSNEYYADSVKGRFTISRDNSKNTLYLQMNSLRPEDTAVYYCARDRIWVDYWGQGTLVTVSSGGGGSGGGGSGGGGSDIQMTQSPDSLAVSLGERATINCKSSQSVLYSSNNKNYLAWYQQKPGQPPKLLIYWASTRESGVPDRFSGSGSGTDFTLTISSLQAEDVAVYYCQQYYSTPLTFGGGTKVEIKTTTPAPRPPTPAPTIASQPLSLRPEACRPAAGGAVHTRGLDFACDIYIWAPLAGTCGVLLLSLVITLYCRSKRSRLLHSDYMNMTPRRPGPTRKHYQPYAPPRDFAAYRSGGGGSGGGGSMVSKGEELFTGVVPILVELDGDVNGHKFSVSGEGEGDATYGKLTLKFICTTGKLPVPWPTLVTTLTYGVQCFSRYPDHMKQHDFFKSAMPEGYVQERTIFFKDDGNYKTRAEVKFEGDTLVNRIELKGIDFKEDGNILGHKLEYNYNSHNVYIMADKQKNGIKVNFKIRHNIEDGSVQLADHYQQNTPIGDGPVLLPDNHYLSTQSALSKDPNEKRDHMVLLEFVTAAGITLGMDELYK

SEQ ID NO: 21 - protein, 777 aa, Artificial Sequence
  HER2 scFv-CD8Hinge-IL2Rbeta tm jm DT-4-1BB- CD3zeta-P2A-GFP (MLB038)
MALPVTALLLPLALLLHAARPDIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGQGTKVEIKGGGGSGGGGSGGGGSGGGGSEVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSTTTPAPRPPTPAPTIASQPLSLRPEACRPAAGGAVHTRGLDFACDIPWLGHLLVGLSGAFGFIILVYLLINCRNTGPWLKKVLKCNTPDPSKFFSQLKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRATNFSLLKQAGDVEENPGPMVSKGEELFTGVVPILVELDGDVNGHKFSVSGEGEGDATYGKLTLKFICTTGKLPVPWPTLVTTLTYGVQCFSRYPDHMKQHDFFKSAMPEGYVQERTIFFKDDGNYKTRAEVKFEGDTLVNRIELKGIDFKEDGNILGHKLEYNYNSHNVYIMADKQKNGIKVNFKIRHNIEDGSVQLADHYQQNTPIGDGPVLLPDNHYLSTQSALSKDPNEKRDHMVLLEFVTAAGITLGMDELYK

SEQ ID NO: 22 - protein, 776 aa, Artificial Sequence
  HER2 scFv-CD8Hinge-IL2Rbeta tm jm DT- CD28- CD3zeta-P2A-GFP (MLB039)
MALPVTALLLPLALLLHAARPDIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGQGTKVEIKGGGGSGGGGSGGGGSGGGGSEVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSTTTPAPRPPTPAPTIASQPLSLRPEACRPAAGGAVHTRGLDFACDIPWLGHLLVGLSGAFGFIILVYLLINCRNTGPWLKKVLKCNTPDPSKFFSQLRSKRSRLLHSDYMNMTPRRPGPTRKHYQPYAPPRDFAAYRSRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRATNFSLLKQAGDVEENPGPMVSKGEELFTGVVPILVELDGDVNGHKFSVSGEGEGDATYGKLTLKFICTTGKLPVPWPTLVTTLTYGVQCFSRYPDHMKQHDFFKSAMPEGYVQERTIFFKDDGNYKTRAEVKFEGDTLVNRIELKGIDFKEDGNILGHKLEYNYNSHNVYIMADKQKNGIKVNFKIRHNIEDGSVQLADHYQQNTPIGDGPVLLPDNHYLSTQSALSKDPNEKRDHMVLLEFVTAAGITLGMDELYK

SEQ ID NO: 23 - protein, 752 aa, Artificial Sequence
  HER2 scFv-CD8Hinge-CD8 tm-4-1BB-CD3zeta-P2A- GFP (MLB079)
MALPVTALLLPLALLLHAARPDIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGQGTKVEIKGGGGSGGGGSGGGGSGGGGSEVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRGSGATNFSLLKQAGDVEENPGPMVSKGEELFTGVVPILVELDGDVNGHKFSVSGEGEGDATYGKLTLKFICTTGKLPVPWPTLVTTLTYGVQCFSRYPDHMKQHDFFKSAMPEGYVQERTIFFKDDGNYKTRAEVKFEGDTLVNRIELKGIDFKEDGNILGHKLEYNYNSHNVYIMADKQKNGIKVNFKIRHNIEDGSVQLADHYQQNTPIGDGPVLLPDNHYLSTQSALSKDPNEKRDHMVLLEFVTAAGITLGMDELYK

SEQ ID NO: 24 - protein, 782 aa, Artificial Sequence
  HER2 scFv-CD8Hinge-SEZ6L2 tm jm-4-1BB-CD3zeta- P2A-GFP (MLB080)
MALPVTALLLPLALLLHAARPDIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGQGTKVEIKGGGGSGGGGSGGGGSGGGGSEVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSTTTPAPRPPTPAPTIASQPLSLRPEACRPAAGGAVHTRGLDFACDLALAILLPLGLVIVLGSGVYIYYTKLQGKSLFGFSGSHSYSPITVESDFSNPLYKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRGSGATNFSLLKQAGDVEENPGPMVSKGEELFTGVVPILVELDGDVNGHKFSVSGEGEGDATYGKLTLKFICTTGKLPVPWPTLVTTLTYGVQCFSRYPDHMKQHDFFKSAMPEGYVQERTIFFKDDGNYKTRAEVKFEGDTLVNRIELKGIDFKEDGNILGHKLEYNYNSHNVYIMADKQKNGIKVNFKIRHNIEDGSVQLADHYQQNTPIGDGPVLLPDNHYLSTQSALSKDPNEKRDHMVLLEFVTAAGITLGMDELYK

SEQ ID NO: 25 - protein, 1033 aa, Artificial Sequence
  841 Claudin18.2 scFv-CD8Hinge-CD8 tm-4-1BB- CD3zeta-P2A-HER2 scFv-IL2Rbeta tm jm DT-CD28-CD3zeta (MLB040)
MALPVTALLLPLALLLHAARPQVQLVQSGAEVKKPGSSVKVSCKASGGTFSSYAISWVRQAPGQGLEWMGRIIPILGIANYAQKFQGRVTVTADKSTSTAYMELSSLRSEDTAVYYCARERDNWDPYYYYYGMDVWGQGTTVTVSSGGGGSGGGGSGGGGSDIQMTQSPSTLSASVGDRVTITCRASQSINSWLAWYQQKPGKAPKLLIYKASSLESGVPSRFSGSGSGTEFTLTISSLQPDDFATYYCQQYNSFPLTFGGGTKVEIKRTTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRGSGATNFSLLKQAGDVEENPGPMALPVTALLLPLALLLHAARPDIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGQGTKVEIKGGGGSGGGGSGGGGSGGGGSEVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSTTTPAPRPPTPAPTIASQPLSLRPEACRPAAGGAVHTRGLDFACDIPWLGHLLVGLSGAFGFIILVYLLINCRNTGPWLKKVLKCNTPDPSKFFSQLRSKRSRLLHSDYMNMTPRRPGPTRKHYQPYAPPRDFAAYRSRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 26 - protein, 994 aa, Artificial Sequence
  841 Claudin 18.2 scFv-CD8Hinge-CD8 tm-4-1BB- CD3zeta-P2A-HER2 scFv-SEZ6L2 tm jm-CD3zeta (MLB108)
MALPVTALLLPLALLLHAARPQVQLVQSGAEVKKPGSSVKVSCKASGGTFSSYAISWVRQAPGQGLEWMGRIIPILGIANYAQKFQGRVTVTADKSTSTAYMELSSLRSEDTAVYYCARERDNWDPYYYYYGMDVWGQGTTVTVSSGGGGSGGGGSGGGGSDIQMTQSPSTLSASVGDRVTITCRASQSINSWLAWYQQKPGKAPKLLIYKASSLESGVPSRFSGSGSGTEFTLTISSLQPDDFATYYCQQYNSFPLTFGGGTKVEIKRTTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRGSGATNFSLLKQAGDVEENPGPMALPVTALLLPLALLLHAARPDIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGQGTKVEIKGGGGSGGGGSGGGGSGGGGSEVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSTTTPAPRPPTPAPTIASQPLSLRPEACRPAAGGAVHTRGLDFACDLALAILLPLGLVIVLGSGVYIYYTKLQGKSLFGFSGSHSYSPITVESDFSNPLYRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 27 - protein, 44 aa, Artificial Sequence
  transmembrane linking juxtamembrane of IL2Rbeta
IPWLGHLLVGLSGAFGFIILVYLLINCRNTGPWLKKVLKCNTPD

SEQ ID NO: 28 - protein, 40 aa, Artificial Sequence
  transmembrane linking juxtamembrane of LDLR
ALSIVLPIVLLVFLCLGVFLLWKNWRLKNINSINFDNPVY

SEQ ID NO: 29 - protein, 54 aa, Artificial Sequence
  transmembrane linking juxtamembrane of SEZ6L
LALAILLPLGLVIVLGSGVYIYYTKLQGKSLFGFSGSHSYSPITVESDFSNPLY

SEQ ID NO: 30 - protein, 249 aa, Artificial Sequence
  841 Claudin 18.2  scFv
QVQLVQSGAEVKKPGSSVKVSCKASGGTFSSYAISWVRQAPGQGLEWMGRIIPILGIANYAQKFQGRVTVTADKSTSTAYMELSSLRSEDTAVYYCARERDNWDPYYYYYGMDVWGQGTTVTVSSGGGGSGGGGSGGGGSDIQMTQSPSTLSASVGDRVTITCRASQSINSWLAWYQQKPGKAPKLLIYKASSLESGVPSRFSGSGSGTEFTLTISSLQPDDFATYYCQQYNSFPLTFGGGTKVEIKRT

SEQ ID NO: 31 - protein, 247 aa, Artificial Sequence
  HER2 scFv
DIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGQGTKVEIKGGGGSGGGGSGGGGSGGGGSEVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSS

SEQ ID NO: 32 - protein, 244 aa, Artificial Sequence
  PD-L1 scFv
EVQLVQSGGGVVQPGRSLRLSCAASGFTFSNYAMHWVRQAPGKGLEWVAIITYAGSNEYYADSVKGRFTISRDNSKNTLYLQMNSLRPEDTAVYYCARDRIWVDYWGQGTLVTVSSGGGGSGGGGSGGGGSDIQMTQSPDSLAVSLGERATINCKSSQSVLYSSNNKNYLAWYQQKPGQPPKLLIYWASTRESGVPDRFSGSGSGTDFTLTISSLQAEDVAVYYCQQYYSTPLTFGGGTKVEIK

SEQ ID NO: 33 - protein, 9 aa, Artificial Sequence
  HA
YPYDVPDYA

SEQ ID NO: 34 - protein, 45 aa, Artificial Sequence
  CD8 Hinge
TTTPAPRPPTPAPTIASQPLSLRPEACRPAAGGAVHTRGLDFACD

SEQ ID NO: 35 - protein, 112 aa, Artificial Sequence
  CD3zeta
RVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 36 - protein, 42 aa, Artificial Sequence
  4-1BB
KRGRKKLLYIFKQPFMRPVQTTQEEDGCSCRFPEEEEGGCEL

SEQ ID NO: 37 - protein, 41 aa, Artificial Sequence
  CD28
RSKRSRLLHSDYMNMTPRRPGPTRKHYQPYAPPRDFAAYRS

SEQ ID NO: 38 - protein, 22 aa, Artificial Sequence
  transmembrane of LDLR
ALSIVLPIVLLVFLCLGVFLLW

SEQ ID NO: 39 - protein, 18 aa, Artificial Sequence
  juxtamembrane of LDLR
KNWRLKNINSINFDNPVY

SEQ ID NO: 40 - protein, 21 aa, Artificial Sequence
  transmembrane of SEZ6L2
LALAILLPLGLVIVLGSGVYI

SEQ ID NO: 41 - protein, 33 aa, Artificial Sequence
  juxtamembrane of SEZ6L2
YYTKLQGKSLFGFSGSHSYSPITVESDFSNPLY

SEQ ID NO: 42 - protein, 24 aa, Artificial Sequence
  CD8 transmembrane (CD8 tm)
IYIWAPLAGTCGVLLLSLVITLYC

SEQ ID NO: 43 - protein, 22 aa, Artificial Sequence
  P2A-1
GSGATNFSLLKQAGDVEENPGP

SEQ ID NO: 44 - protein, 19 aa, Artificial Sequence
  P2A-2
ATNFSLLKQAGDVEENPGP

SEQ ID NO: 45 - protein, 239 aa, Artificial Sequence
  GFP
MVSKGEELFTGVVPILVELDGDVNGHKFSVSGEGEGDATYGKLTLKFICTTGKLPVPWPTLVTTLTYGVQCFSRYPDHMKQHDFFKSAMPEGYVQERTIFFKDDGNYKTRAEVKFEGDTLVNRIELKGIDFKEDGNILGHKLEYNYNSHNVYIMADKQKNGIKVNFKIRHNIEDGSVQLADHYQQNTPIGDGPVLLPDNHYLSTQSALSKDPNEKRDHMVLLEFVTAAGITLGMDELYK

SEQ ID NO: 46 - protein, 21 aa, Artificial Sequence
  Leader Sequence
MALPVTALLLPLALLLHAARP

SEQ ID NO: 47 - protein, 124 aa, Artificial Sequence
  VH of hCLDN18.2-808 antibody
EVQLVQSGGGVVQPGRSLRLSCAASGFTFSSYAMHWVRQAPGKGLEWVAVISYDGSNKYYADSVKGRFTISRDNSKNTLYLQMNSLRAEDTAVYYCARELRFFDWLLGSAFDIWGQGTTVTVSS

SEQ ID NO: 48 - protein, 107 aa, Artificial Sequence
  VL of hCLDN18.2-808 antibody
DIVMTQSPSSLSASVGDRVTITCRASQGIRNDLGWYQQKPGKAPKLLIYAASSLQSGVPSRFSGSGSGTDFTLTISSLQPEDFATYYCLQDYNYPRTFGQGTKVEIK

SEQ ID NO: 49 - protein, 125 aa, Artificial Sequence
  VH of hCLDN18.2-841 antibody
QVQLVQSGAEVKKPGSSVKVSCKASGGTFSSYAISWVRQAPGQGLEWMGRIIPILGIANYAQKFQGRVTVTADKSTSTAYMELSSLRSEDTAVYYCARERDNWDPYYYYYGMDVWGQGTTVTVSS

SEQ ID NO: 50 - protein, 107 aa, Artificial Sequence
  VL of hCLDN18.2-841 antibody
DIQMTQSPSTLSASVGDRVTITCRASQSINSWLAWYQQKPGKAPKLLIYKASSLESGVPSRFSGSGSGTEFTLTISSLQPDDFATYYCQQYNSFPLTFGGGTKVEIK